(19) United States
(12) Patent Application Publication
(10) Pub. No.: US 2023/0165841 A1
(43) Pub. Date: Jun. 1, 2023

(54) PREVENTION AND INTERVENTION OF INFARCT EXPANSION FOLLOWING HEMORRHAGIC INFARCTIONS

(71) Applicant: CEDARS-SINAI MEDICAL CENTER, Los Angeles, CA (US)

(72) Inventor: Rohan DHARMAKUMAR, Moorpark, CA (US)

(73) Assignee: CEDARS-SINAI MEDICAL CENTER, Los Angeles, CA (US)

(21) Appl. No.: 17/912,227

(22) PCT Filed: Mar. 19, 2021
(86) PCT No.: PCT/US2021/023292

§ 371 Date: Sep. 16, 2022

Related U.S. Application Data
(60) Provisional application No. 62/992,832, filed on Mar. 20, 2020.

(51) Int. Cl. A61K 31/4412; A61P 9/10; A61K 31/165; G01N 33/68

(52) U.S. Cl. CPC A61K 31/4412; A61P 9/10; A61K 31/165; G01N 33/6887; G01N 2333/4712; G01N 2800/52

(57) ABSTRACT

Methods of treating a subject with myocardial infarction are provided, which include selective targeting time-dependent iron products at different phases of the infarction. It is discovered that during acute phase of myocardial infarction, ferrous iron in the form of heme accumulate, often followed by infarct expansion, and during the chronic phase, ferric iron in the form of crystals are dominant Chelator agents specific for ferrous iron, heme or ferric iron are demonstrated in the protection of cardiomyocytes, reduction of infarct expansion, or improving cardiac remodeling following myocardial infarction. Also provided are methods for determining the presence of intramyocardial hemorrhage by measuring plasma level of cardiac troponin before and after re-vascularization or a reperfusion therapy, which can be used to guide therapeutic treatment or intervention procedures to control the hemorrhage and mitigate infarct expansion.

### CROSS-REFERENCE TO RELATED APPLICATIONS

This application includes a claim of priority under 35 U.S.C. § 119(e) to U.S. provisional patent application No. 62/992,832, filed Mar. 20, 2020, the entirety of which is hereby incorporated by reference.

### STATEMENT REGARDING FEDERALLY SPONSORED RESEARCH OR DEVELOPMENT

This invention was made with Government support under grant numbers HL133407 and HL147133 awarded by National Institutes of Health. The Government has certain rights in the invention.

### FIELD OF INVENTION

This invention relates to targeted therapeutics for intervention of myocardial infarction and/or prevention of infarct expansion, and in some embodiments following intramyocardial hemorrhage with infarctions.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Reperfusion therapy, particularly percutaneous coronary intervention (PCI), is instrumental in saving patients from immediate death from acute myocardial infarction (MI). Restoring blood flow (reperfusion) to blocked epicardial coronary arteries has reduced immediate death from acute myocardial infarction (MI). However, over the same period since PCI has become the mainstay for treatment of acute MI, the incidence of post MI heart failure has become epidemic. The functional recovery of the heart following a reperfused MI is variable, with some hearts accelerating towards heart failure, while others not so. Noninvasive imaging has been instrumental in identifying patients with reperfused hemorrhagic MIs as the ones at the greatest risk of extensive adverse LV remodeling and heart failure. Center for Disease Control reports that >300,000 deaths/year are attributable to chronic heart failure (CHF) in the US. The terminal recourse of these patients is heart transplantation, but transplantation is limited by availability of donor hearts, eligibility and cost. The size of MI is a long-established predictor of CHF, and recent advances in imaging have shown that hemorrhage, a potential consequence of reperfusion therapy for MI, is a major predictor of major adverse cardiovascular events (MACE). Several studies have shown that hemorrhagic MI patients are at >2-fold greater risk for MACE than those without hemorrhage. Yet, why hemorrhagic MIs carry the greatest risk of developing heart failure was unclear.

Hemorrhagic (MIs) are commonly observed after coronary artery reperfusion. It is estimated that intramyocardial hemorrhage (IMH) is observable in nearly in half of the patients successfully revascularized for acute MI (AMI). Hemorrhagic AMIs are associated with adverse LV remodeling and poor prognosis in the ensuing chronic phase of MI compared to AMIs without IMH. In the heart, past studies have shown that large reperfused MIs often have hemorrhage, and reperfusion injury in the acute phase of leads to oxidative stress and ultimately cardiomyocyte death.

The present invention looks into the time-dependent changes in the hemorrhagic MI zones, identifies correlational or causal role of hemorrhagic MI in LV remodeling, and provides therapeutic agents and dosing regimen to reduce infarct expansion following hemorrhagic infarction.

### SUMMARY OF THE INVENTION

The following embodiments and aspects thereof are described and illustrated in conjunction with compositions and methods which are meant to be exemplary and illustrative, not limiting in scope.

Methods of treating a subject having been diagnosed with or showing symptoms of myocardial infarction by reducing infarct size from the acute phase are provided, including administering to the subject an effective amount of a ferrous iron chelator, an agent that binds heme or an agent that regulates heme during the acute phase of the myocardial infarction. Further embodiments of the methods for treating the symptoms of myocardial infarction and/or improving myocardial remodeling are provided, which include administering to the subject an effective amount of a ferrous iron chelator, an agent that binds heme or an agent that regulates heme during the acute phase of the myocardial infarction, and administering to the subject an effective amount of a ferric iron chelator during the chronic phase of the myocardial infarction.

In some embodiments, the subject has intramyocardial hemorrhage with the myocardial infarction, and the subject is selected for administration of the ferrous iron chelator, the agent that binds heme, the agent that regulates heme or the ferric iron chelator during the acute phase of the infarction. In some embodiments, the subject has had reperfusion before the administration of the ferrous iron chelator, the agent that binds heme, the agent that regulates heme or the ferric iron chelator. In further embodiments, the administration of the ferrous iron chelator, the agent that binds heme, or the agent that regulates heme is before reperfusion; during reperfusion; after reperfusion; both before reperfusion and after reperfusion within the acute phase of the infarction; or a combination including during reperfusion. Further embodiments provide administering an effective amount of a ferrous iron chelator or an agent that binds or regulates heme to a subject, wherein the subject is no more than about 3 days from the onset of myocardial infarction.

Various embodiments show that the acute phase of the myocardial infarction is within about 3 days of onset of the myocardial infarction. Various embodiments show the administration of the ferrous iron chelator, the agent that binds heme, or the agent that regulates heme when there is no ferric iron in or near the myocardium infarct.

Various embodiments show a convalescent period is after the acute phase, hence beginning after 3 days of onset of infarction. In some embodiments, the chronic phase begins after the acute phase, or can be characterized by evidence of presence of ferric iron in or near the myocardium infarct. In some embodiments, chronic myocardial infarction is over 2, 3, 4, 5, 6 months, or 1 year, or longer.

Exemplary ferrous iron chelators, agents that bind heme or agents that regulate heme, or suitable for administration during the acute phase of myocardial infarction, include but are not limited to dexrazoxane (ICRF-187), 2,2-bipyridl, hemopexin, a heme oxygenase, hinokitiol. Other suitable heme binding agents include haptoglobin, albumin, ferritin, α1-microglobulin, α1-antitrypsin, glutathione-S-transferase, liver fatty acid binding protein, heme-binding protein 23 (also known as peroxiredoxin), p22 heme binding protein, and glyceraldehyde-3-phosphate dehydrogenase. Exemplary agents that regulate heme are (a) heme degrading proteins, such as heme oxygenase 1 (HO-1) and nuclear factor E2 related factor 2 (Nrf2); or (b) factors that increase the amount of heme-binding proteins or heme-degrading proteins, such as feline leukemia virus subgroup C receptor 1a (FLVCR1a), FLVCR2, and ATP-binding cassette subfamily G member 2 (ABCG2).

Exemplary ferric iron chelators include but are not limited to desferrioxamine (also known as deferoxamine), deferiprone, deferasirox, hinokitiol, pyridoxal isonicotinoyl hydrazone, salicylaldehyde isonicotinoyl hydrazone, exochelins (including desferri-exochelins). Desferri-exochelins are hexadentate molecules, and by forming a one-to-one binding relationship with iron, they prevent free radical reactions; whereas deferiprone is a bidentate molecule and desferasirox is a tridentate molecule.

Further embodiments of the methods include administering one or more anti-inflammatory agents to a subject with hemorrhagic infarction in the acute phase and/or the chronic phase. An exemplary anti-inflammatory agent is colchicine.

In some embodiments, the methods for treating a subject in need thereof include selecting a subject in an acute phase of a myocardial infarction, optionally having undergone or to be treated with reperfusion, and subsequently administering the ferrous iron chelator, the agent that binds heme, or the agent that regulates heme in the acute phase, optionally followed by administering the ferric iron chelator in the chronic phase.

In other embodiments, the methods for treating a subject in need thereof include selecting a subject having intramyocardial hemorrhage during acute myocardial infarction, and administering the ferrous iron chelator, the agent that binds heme, or the agent that regulates heme in the acute phase, optionally followed by administering the ferric iron chelator and in some embodiments coupled with an anti-inflammatory agent in the chronic phase.

A method is provided for treating a subject having been diagnosed with or showing symptoms of myocardial infarction by preventing or reducing infarct expansion, which includes administering an effective amount of a ferrous iron chelator, an agent that binds heme or an agent that regulates heme during the acute phase (or within 3 days from onset) of the myocardial infarction. In a further embodiment of this method, an effective amount of a ferric iron chelator is further administered during the chronic phase of the myocardial infarction.

In one aspect, the subject in the method has experienced reperfusion hemorrhage. In another aspect, the subject in the method has experienced or is undergoing infarct expansion. In yet another aspect, the subject in the method is undergoing infarct expansion following hemorrhagic infarction. In a further aspect, the subject has intramyocardial hemorrhage after acute myocardial infarction.

Methods are also provided for reducing myocardial infarct size, and/or inhibiting expansion of the myocardial infarct size, in a subject in need thereof, which include administering a composition comprising an effective amount of a ferrous iron chelator, an agent that binds heme, an agent that regulates heme, or a combination thereof, during the acute phase or within 3 days of the onset of myocardial infarction; measuring a blood level of troponin or cardiac troponin of the subject before and after coronary re-vascularization or reperfusion therapy, or at two or more time points after the coronary re-vasucularization or the reperfusion therapy; and administering a treatment to the subject to control hemorrhage from the cardiac chamber of the subject, when the blood level of troponin or cardiac troponin quickly and sharply rises within 18 hours following the coronary re-vascularization or the reperfusion therapy, or to an increase that is at least 1.5 ng/mL greater within these 18 hours than the baseline level before the reperfusion, or rises by a rate of at least 0.4 ng/mL/hr within 12 hours following the reperfusion.

Also provided are methods for treating hemorrhagic myocardial infarction in a subject, and/or mitigating infarct expansion in a subject with hemorrhagic myocardial infarction comprises: administering an effective amount of a ferrous iron chelator, an agent that binds heme, or an agent that regulates heme during the acute phase of the myocardial infarction to the subject, optionally further administering an effective amount of a ferric iron chelator after the acute phase to the subject, who has been determined to have a blood level of troponin that peaks within 18 hours after the subject receives a reperfusion therapy, or who has been determined to have a blood level of troponin that increases by at least 1.5 ng/mL or greater within 18 hours following the reperfusion therapy compared to a level before the reperfusion therapy, or who has been determined to have an increased level of troponin by a rate of at least 0.4 ng/mL/hr within 12 hours following the reperfusion therapy.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 is a flow chart detailing the study timeline in Example 5 about the animal groups, time points of cardiac MRI, euthanasia and histology. The left-hand side details the observational study, and the right-hand side details the interventional study.

FIGS. 2A and 2B depict that lipomatous metaplasia in early and late chronic phase of MI depends of iron concentration in the acute phase of MI. FIG. 2A is a bar graph showing the mean R2* and proton density fat fraction (PDFF) in hemorrhagic (IMH+) and non-hemorrhagic (IMH−) territories relative to remote regions at D3, Wk8 and M6. Relative R2* for IMH+ was higher than R2* of IMH− and remained unchanged between D3 and M6. However, during the same time relative PDFF increased with time in MIs with IMH but this was not evident in MIs without IMH. FIG. 2B is a scatter plot showing the relation between relative PDFF and relative R2* as determined on D3, Wk8 and M6. Results from linear regression analysis are shown in the inset legend. Lines of best fit from regression analysis between Relative PDFF and Relative R2* at D3 (lowest line at relative R2*=2; y=0.32+0.86 xx), Wk8 (the middle line at relative R2* close to 2; y=−1.42+2.52 xx) and M6 (the highest line at relative R2*=2; y=−2.94+3.74 xx) are shown. (*) represents well-known off-resonance artifacts in non-MI (posterior wall) regions.

FIG. 2C shows cardiac MRI images, relating to FIGS. 2A and 2B, of representative, raw and processed, short-axis late-gadolinium enhancement (depicting zone of MI), R2* (depicting iron concentration) and PDFF (depicting fat fraction) from an animal at day 3 (D3), week 8 (Wk8) and month 6 (M6) post MI.

FIGS. 2D and 2E depict that lipomatous metaplasia in the early chronic phase of MI is unique to hemorrhagic MIs and is observed exclusively at the confluence of iron and lipid remnants. FIG. 2D shows microscopic images of serial paraffin sections from an 8-week-old hemorrhagic MI stained with elastin-modified Masson's trichrome (EMT) (left-hand column), H&E (middle column) and Prussian Blue (PB) stains (right-hand column) from a zone of peri-infarct zone of sub-endocardium, where the zoomed-in areas labeled by a and 13 in the upper-row images are shown in the middle-row and lower-row images in the same column. Zoomed-in areas are indicated by rectangles outlined by dotted line in respective images in the upper row. Individual foam cells were exclusively observed in the in the pen-infarct and border zones of hemorrhagic MIs and exclusively co-localized with residual iron deposits. Scale bar in images of FIG. 2D is 500 μm. FIG. 2E shows serial frozen sections from an 8-week-month-old hemorrhagic MI stained with EMT, H&E, Oil-Red-O (ORO) and PB stains. The upper row shows that foam cells were observed only at the confluence of iron (PB stained regions) and lipid deposits (ORO regions). In contrast, iron+/lipid-regions (the middle row) as well as the iron-/lipid+(the lower row) regions did not exhibit LM. Scale bar in images in FIG. 2E equals 100 μm.

FIGS. 2F and 2G depict lipomatous metaplasia in the early chronic phase of MI is unique to hemorrhagic infarcts and is observed exclusively at the have confluence of iron and lipid. FIG. 2F shows microscopic images on serial paraffin sections from an 8-week-old hemorrhagic MI stained with elastin-modified Masson's trichrome (EMT) (left column), H&E (middle column) and Prussian Blue (PB) (right column) stains from a zone of pen-infarct zone of sub-endocardium (upper two rows) and midmyocardium (lower two rows), scale bar=500 μm. Individual foam cells were exclusively observed in the in the pen-infarct and border zones of hemorrhagic MIs and exclusively co-localized with residual iron deposits. FIG. 2G shows microscopic images on serial frozen sections from an 8-week-month-old hemorrhagic MI stained with Oil-Red-O (ORO) and PB stains, scale bar=100 μm. Foam cells were observed only at the confluence of iron (PB stained regions) and lipid deposits (ORO regions). This indicates that even small traces of iron deposits from reperfusion hemorrhage, if in contact with lipid remnants from necrotic myocardium, carry a risk for LM.

FIG. 2H depicts that the absence of foam cells and lipomatous metaplasia in the early chronic phase of non-hemorrhagic MI is linked to absence of reperfusion hemorrhage in the acute phase of MI. On the left-hand column, serial paraffin sections from an 8-week-old non-hemorrhagic MI stained with elastin-modified Masson's trichrome (EMT), H&E and Prussian Blue (PB), scale bar=500 μm. Non-hemorrhagic MIs were consistently negative for iron and foam cells in the early phase of chronic MI. On the right-hand column, foam cell-negative scars consistently exhibited traces of ORO-stained lipid remnants in contiguous frozen sections, scale bar=100 μm. This data indicates an important role for iron deposits in triggering lipid peroxidation, foam cell formation, LM and adverse ventricular remodeling.

FIG. 2I depicts that foam cell formation in the early chronic phase of hemorrhagic MI is accompanied by highly localized deposition of ceroid lipopigment in iron-rich MI zones. Histological (upper row) and confocal microscopy (middle and lower rows) evaluations of the 8-week-old hemorrhagic MI are shown. Serial paraffin sections of the infarcted subendocardial myocardium at 8-weeks post-MI were stained with elastin-modified Masson's trichrome stain (EMT), H&E and Prussian Blue (PB). Dotted line boxes/rectangles (images in second row) are shown as zoomed-in regions (images in third row). Consistent with FIGS. 2D and 2E, the extensive co-localization of fat (foam cells) with persistent iron deposits. Evidence of ceroid was determined on the basis of autofluorescence in sections stained with PB by confocal microscopy and are shown in panel PB-1 at excitation wavelength of 405 nm and emission wavelength of 428-496 nm. Panel PB-2 represents a Differential Interference Contrast (DIC) for PB-1. Panel PB-3 shows an overlay of PB-1 autofluorescence and DIC (PB-2). There is extensive co-localization of ceroid with iron deposits and foam cells. Scale bar equals 100 μm.

FIG. 2J depicts that iron-rich scar regions undergoing lipomatous metaplasia exhibit perpetual macrophage ingress, M1 macrophage polarization, foam cell formation and expansion of “death zone” in the early phase of chronic hemorrhagic MI. Representative serial paraffin histology sections from 8-week-old hemorrhagic MI were stained with elastin-modified Masson's trichrome (EMT) stain, H&E, Prussian Blue (PB), as well as the anti-Cleaved Caspase 3 (CC3), anti-MAC387, anti-E06, anti-IL-113, anti-TNF-α, anti-MMP-9, anti-CD163, anti-CD36, and anti-GLUT-1 antibodies. Autofluorescence of ceroid was examined in sections stained with PB stain and E06 antibody. (PB-1 and E06-1) Excitation wavelength: 405 nm and Emission wavelength: 428-496 nm. (PB-2 and E06-2) Differential Interference Contrast (DIC). (PB-3 and E06-3) Overlay. There is extensive co-localization of ceroid with iron deposits (PB) and foam cells. Positive immunohistochemical (IHC) staining with anti-CC3 antibody confirmed the ongoing apoptosis of siderophage-derived foam cells (arrows). Positive IHC staining with MAC387 antibody indicates that new macrophages are perpetually recruited to the regions with apoptotic siderophage-derived ceroid-rich foam cells. There is also the extensive co-localization of ceroid with E06-stained oxidized phospholipids (E06) in and foam cell-rich regions. Positive staining for proinflammatory macrophage markers (IL1-beta, TNF-alpha and MMP-9) indicates that siderophages in the ceroid-rich regions preferentially polarize to pro-inflammatory M1 phenotype. CD163-positive staining in iron-rich regions undergoing LM indicates perpetual iron-induced macrophage induction and siderophage-to-foam cell transformation. Intense staining with CD36 antibody confirmed the presence of foam cells. Glycolytic M1 macrophage phenotype in macrophages undergoing foam cell transformation was also demonstrated by intense immunoreactivity for GLUT-1. Scale bar equals 50 am.

FIG. 2K depicts mast cells home to iron-laden regions of scar tissue undergoing lipomatous metaplasia in the early phase of chronic hemorrhagic MI. Representative serial histology sections from 8-week-old hemorrhagic MI were stained with elastin-modified Masson's trichrome (EMT) stain, H&E, Prussian Blue (PB), Toluidine Blue (TB) as well as the anti-Cleaved Caspase 3 (CC3), anti-MAC387, anti-E06, anti-IL-113, anti-TNF-α, anti-MMP-9, anti-CD163, anti-CD36, and anti-GLUT-1 antibodies. Note the extensive co-localization of iron deposits (PB) and foam cells. Positive immunohistochemical (IHC) staining with anti-CC3 antibody confirmed the ongoing apoptosis of siderophage-derived foam cells (arrows). IHC staining with MAC387 antibody indicates that new macrophages are perpetually recruited to the regions with apoptotic siderophage-derived ceroid-rich foam cells. Glycolytic M1 macrophage phenotype in macrophages undergoing foam cell transformation was demonstrated by intense immunoreactivity for GLUT-1 and other proinflammatory macrophage markers (IL1-beta, TNF-alpha and MMP-9); which indicates that siderophages in the ceroid-rich regions preferentially polarize to pro-inflammatory M1 phenotype. CD163-positive staining in iron-rich regions undergoing LM indicates perpetual iron-induced macrophage induction and siderophage-to-foam cell transformation. Intense staining with CD36 antibody confirmed the presence of foam cells. Note also the extensive co-localization of E06-stained oxidized phospholipids (E06) with foam cells in siderophage-rich regions. Positive IHC staining with anti-CC3 antibody confirmed the ongoing apoptosis of siderophage-derived foam cells (arrows). Note that iron- and siderophage-rich zones in the vicinity of scar regions undergoing LM exhibited increased homing and degranulation of mast cells as evident by TB staining. Note the individual degranulated mast cells in TB1-6. Scale bar equals 50 am.

FIGS. 2L and 2M depict lipomatous metaplasia in the late chronic phase of MI is unique to hemorrhagic Infarcts. FIG. 2L shows serial paraffin sections from a 6-month-old hemorrhagic MI stained with elastin-modified Masson's trichrome (EMT), H&E and Prussian Blue (PB) stains, scale bar=500 μm. Larger fat depots typically penetrated scar tissue at its internal core (zone α) were observed. Notably, these larger foam cell clusters typically colocalized with iron deposits along the fat depot periphery while the core of the growing adipose tissue contained traces of iron deposits (zone β, arrows). FIG. 2M shows serial frozen sections from a dog with 6-month-old hemorrhagic MI stained with H&E, EMT, Oil-Red-O (ORO) and PB stains, scale bar=100 μm. Note the extensive colocalization of iron deposits and foam cells in the fat depot penetrating the internal core of hemorrhagic scar.

FIGS. 2N and 2O depict reperfusion hemorrhage-derived iron deposition carries a risk of lipomatous metaplasia in late chronic phase of post-MI scar. FIG. 2N shows serial paraffin sections, representing subendocardial region, from a 6-month-old hemorrhagic MI stained with elastin-modified Masson's trichrome (EMT), H&E and Prussian Blue (PB) stains, scale bar=500 μm. Individual foam cells were exclusively observed in the pen-infarct and border zones and exclusively co-localized with chronic iron deposits. FIG. 2O in upper two rows shows serial paraffin sections, representing midmyocardial region, from a 6-month-old hemorrhagic MI stained with elastin-modified Masson's trichrome (EMT), H&E and Prussian Blue (PB) stains, scale bar=500 μm. Larger fat depots typically penetrated scar tissue at its internal core. These larger foam cell clusters typically colocalized with iron deposits along the fat depot periphery while the core of the growing adipose tissue contained traces of iron deposits. FIG. 2O in the lower two rows shows serial frozen sections from a dog with 6-month-old hemorrhagic MI stained with Oil-Red-O (ORO) and PB stains, scale bar=50 μm. Individual foam cells emerged exclusively at the confluence of iron deposits and lipid remnants from necrotic myocardium. It supports the notion that even traces of iron deposits from reperfusion hemorrhage, if in contact with lipid remnants from necrotic myocardium, carry a risk for LM in old hemorrhagic scars.

FIG. 2P depicts lipomatous metaplasia in the late chronic phase of hemorrhagic MI is unique to scarred regions with iron deposits and lipid remnants. The first two rows show serial frozen sections from an 6-month-old hemorrhagic MI stained with H&E, elastin-modified Masson's trichrome (EMT), Oil-Red-O (ORO), and Prussian Blue (PB) stains, scale bar=500 μm. A peri-infarct zone of sub-endocardium is presented in dotted rectangles. There is extensive colocalization of iron deposits with mini clusters of fat. In contrast, regions without iron but with lipids (the third and fourth rows) as well as regions with iron but no lipids (the last row) did not exhibit LM; scale bar=100 μm. Importantly, the last row of images indicate that iron deposits and lipid remnants must be in the immediate proximity for LM to take place. Note the absence of foam cells/LM in this region with iron despite the fact that lipids are “close-by” but not in contact with iron remnants.

FIG. 2Q depicts lipomatous metaplasia is also not evident in in the late chronic phase of non-hemorrhagic MI. In the left-hand column, serial paraffin sections from an 6-month-old non-hemorrhagic MI stained with elastin-modified Masson's trichrome (EMT), H&E and Prussian Blue (PB) are presented, scale bar=500 μm. Old non-hemorrhagic MIs were consistently negative for iron, foam cells and lipomatous metaplasia. In the right-hand column, the foam cell- and LM-negative scars consistently exhibited traces of ORO-stained lipid remnants in contiguous frozen sections within old scars (scale bar=100 μm) indicates that iron plays an important role in driving lipomatous metaplasia and fatty degeneration of infarcted myocardium.

FIG. 2R depicts siderophage-derived foam cell formation within 6-month old hemorrhagic scars is accompanied by intracellular accumulation of ceroid. TEM images are shown of a macrophage cell (first row, left image), it being with Fe and lipid granules (first row, middle image. Elemental map of Fe distribution within that area is shown in the first row, right-hand side image. Typical EDS spectra collected from the lipid and iron area of the cell are shown in the two spectra. The intracellular ceroids were observed as clusters of ring structures. Electron-dense precipitates were formed and visualized within these rings. To confirm that these electron-dense precipitates contained iron, sections were subjected to electron-dense spectroscopy (EDS), which showed that the sites containing the electron-dense precipitates had a strong iron peak. Moreover, as evident in panel e, these iron precipitates within the macrophages were highly co-localized with extensive lipid rich regions of the cell. Iron and lipid globules were not detectable in non-hemorrhagic MI zone, based on TEM images and EDS spectra analysis.

FIG. 2S depicts the inability of siderophages to switch from M1 to M2 phenotype is coincident with foam cell formation and lipomatous metaplasia in the late chronic phase of hemorrhagic MI. Representative serial paraffin histology sections from 6-month-old hemorrhagic MI were stained with H&E, elastin-modified Masson's trichrome (EMT), Prussian Blue (PB), Toluidine Blue (TB) as well as the anti-MAC387, anti-E06, anti-Cleaved Caspase 3 (CC3), anti-CD36, anti-CD163, anti-MMP-9, anti-TNF-α and anti-IL-113 antibodies are shown. Persistent iron deposits co-localized with fat depots within old hemorrhagic scar. As observed in the early phase of chronic MI, increased homing of mast cells (TB, arrows) in iron-laden regions undergoing LM was also evident in the late chronic phase of MI. Note the individual degranulated mast cells in TB1-4. Increased generation of oxidized phospholipids in the “iron-mast cell-macrophage-fat territory” was evidenced by intense E06 staining. Positive immunohistochemical staining with anti-CC3 antibody confirmed the ongoing apoptosis of siderophage-derived foam cells (arrows). New macrophages (MAC387+) were persistently recruited to the E06+ regions undergoing LM. Retention of the glycolytic phenotype in macrophages undergoing foam cell transformation was confirmed by intense immunoreactivity for GLUT-1. Fat depots within “iron-mast cell territory” stained positive for CD36 indicating macrophage-derived foam cell formation. Note the individual foam cells in CD36-A&B. CD163-positive macrophages co-localized with foam cells indicating iron-induced macrophage-to-foam cell transformation. Positive staining for MMP-9, TNF-alpha and IL-10 in the “iron-mast cell-macrophage-fat territory” indicates that hemorrhagic infarcts lead to prolonged mast cell-mediated inflammatory response culminating in fatty degeneration of post-MI scar. Scale bar equals 50 μm.

FIGS. 3A-3C depict reduction of residual iron by deferiprone in the post MI period is accompanied by reduction in lipomatous metaplasia in canine models of hemorrhagic MI. FIG. 3A is a bar graph showing the residual iron concentration based on R2* in animals with hemorrhagic MI undergoing DFP treatment and no treatment (normalized to values obtained on D3) at Wk8 and M6. Note the marked reduction in the residual iron at Wk8 and M6 in the treated group compared to the untreated group. FIG. 3B is a bar graph showing the extent of fat infiltration based on PDFF in animals with hemorrhagic MI undergoing DFP treatment and no treatment (normalized to values on D3) at Wk8 and M6. Note the marked reduction in the fat content at Wk8 and M6 in the treated group compared to untreated group. FIG. 3C shows representative, raw and processed, short-axis late-gadolinium enhancement (depicting zone of MI), R2* (depicting iron concentration) and PDFF (depicting fat concentration) cardiac MRI images from one animal with hemorrhagic MI and receiving DFP treatment (DFP+/IMH+) and another animal with hemorrhagic MI but not receiving DFP treatment (DFP−/IMIH+), acquired on day 3 (D3), week 8 (Wk8) and month 6 (M6) post MI are shown. Note the reduction in R2* within the infarction zone in the treated animal at Wk8 and M6, relative to D3. In the untreated animal, R2* was elevated on D3 and remained elevated at Wk8 and M6. Also note that in the DFP treated animal, the infiltration of fat within the MI zone was visibly reduced compared to in the untreated animal at Wk8 and M6. (*) represents well-known off-resonance artifacts in non-infarcted (posterior wall) regions.

FIGS. 4A-4F depict orally administered deferiprone significantly improves structural LV remodeling in canine models of hemorrhagic MI. Structural remodeling based on changes in diastolic wall thickness of remote region, MI region and composite remodeling (indexed as a ratio of infarct/remote wall thickness) in treated (DFP+/IMH+) and untreated (DFP−/IMH+) animals (with matched MI size and iron concentration as determined on LGE and R2* cardiac MRI on day 3) at day 3 (D3), week 8 (Wk8) and month 6 (M6) are shown. FIGS. 4A, 4C and 4E show the absolute values of diastolic wall thickness at each of the time points. FIGS. 4B, 4D and 4F show the rate of change in structural indices between D3 to Wk8 (duration over which the DFP+/IMH+ group received DFP treatment, but not the DFP−/IMIE1+ group), Wk8 to M6 (duration over which neither the DFP+/IMH+ nor DFP−/IMH+ groups received any DFP), and D3 to M6 (the full study period). DFP treated animals demonstrated positive structural remodeling compared to the untreated controls.

FIGS. 4G-4L depict orally administered deferiprone significantly improves functional LV remodeling in canine models of hemorrhagic MI. The corresponding functional LV remodeling based on changes peak circumferential strain, end-systolic volume and LV ejection fraction in the same animals over the same time intervals is shown. Baseline data (BL, acquired prior to MI), is shown for reference. Both structural (FIGS. 4A-4F) and functional (FIGS. 4G-4L) LV remodeling show more beneficial in DFP+/IMH+ group compared to DFP−/IMH+ group, which shows adverse remodeling towards heart failure. * denotes p<0.05, ** p<0.005.

FIG. 5 is a schematic of types of reperfused acute MI, fractions of MIs with hemorrhage, and associated MACE risk. Myocyte death proceeds from the subendocardium as a “wave” of injury with increasing ischemic time. Key features of different MIs are: Type 1: early, reperfusion with myocyte injury only; Type 2: myocyte injury with mild MO; Type 3: myocyte injury with late MO. Zone A: myocyte injury only; Zone B: myocyte injury and mild MO (some slow flow); Zone C: myocyte injury with late MO (no flow). Hemorrhage occurs ˜75% of the time in Zone C. hMIs have the largest 6-month MACE risk (16% vs. 7%) among all MI types.

FIG. 6 is a schematic showing time-dependent transformation of hemorrhage and its effects on the heart in the super-acute, acute, sub-acute and chronic phases post MI. Super-Acute (hours): the initial MI zone (outlined in the light circle) is about the area of the zone of IMH (outlined in the bold circle); Acute (days): Amplified ROS activity from excessive heme in MI zone->ferroptosis, which is infarct expansion; Sub-acute (1-2 weeks): transition of Fe′->Fe′ via Fenton Reaction (FR) in mitochondria, Fe′ as end-product of FR (extracellular); Chronic (months): persistent pro-inflammatory burden, which leads to adverse LV remodeling.

FIG. 7 is a schematic showing that acute damage from hemorrhage within MI confers infarct expansion via ferroptosis-mediated cell death of cardiomyocytes and Fe′ accumulation in MI zone. In the box depicts that hemorrhage drives ferroptosis-mediated cell death of cardiomyocytes, including I-IV. I. Heme accumulation: lysis of red cells release heme, heme uptake by cardiomyocytes, and breakdown of heme release Fe²⁺; II. Excessive ROS: Fenton reaction produces ROS; III. Mitochondrial Damage: chain reaction of ROS with mitochondrial membrane; IV: Cardiomyocyte death: Fe³⁺ release. Followed by 48-72 hours, infarction expands beyond the area at risk.

FIG. 8 depicts reduction in LV remodeling following delayed DFP, but not EDTA, treatment. Rats with hMI received delayed DFP treatment (DFP+) post MI had reduced structural, compositional and inflammatory remodeling compared to EDTA or PBS. Gross short-axis fixed sections show marked thinning of MI sections, LV dilation in PBS and EDTA treated rats compared to DFP treated rats. On short-(SAx) and long-axis (LAx), T*2-CMR obtained from rats, iron deposits and LV remodeling were reduced (thicker walls; reduced LV dilation) compared to matched controls receiving EDTA or PBS. Masson's Trichrome (EMT) confirmed CMR findings of wall thickness in scarred and remote areas. Prussian Blue (PB) confirmed that DFP+ rats had much lower iron in MI zone versus EDTA- and PBS-treated rats. Histochemistry sections (EMT) showed reduced proinflammatory markers (IL-113, TNF-α, MMP9) within scar.

FIG. 9 is a summary of treatment protocols for Examples 3-1 and 3-2. Rats will undergo 90 min-I/R protocol; hMIs confirmed with CMR in <1 hr post reperfusion. Prevention arm (Task 1): placebo treatment (Grp 1); specific ICTs (Grps 2-4); hemopexin (Hx, Grp 5). Reduction arm (Task 2): delayed treatment (e.g., earliest start time determined Example 2)-placebo treatment (Grp 6); specific ICTs (Grps 7-8); hemopexin (Hx; Grp9). Rats will be followed with CMR and sacrificed for histology on week 8. Time window for treatment: (1) Prevention Arm: Initiated immediately after first CMR (<1 hr post reperfusion) with ferrous chelator (DXZ) and with or without ferric chelator (DFP) at the earliest point of iron crystallization identified on Aim 2. (2) Reduction Arm: Initated at earliest point of iron crystallization identified on Aim 2. Acronyms: cardiac MRI (CMR), iron chelation therapy (ICT). In some embodiments, the ICT also includes deferoxamine.

FIGS. 10A-10C depict infarct size and iron content within MI depend on type of MI. FIG. 10A shows mean acute MI size (% LV) for each group. FIG. 10B shows iron content of each MI type (IMH−, IMH+ and NR) and non-infarcted tissue. FIG. 10C shows representative MRI (LGE and T2*) of reperfused [with (IMH+) and without (IMH−) hemorrhage] and non-reperfued (NR) MIs.

FIG. 10D depicts rat model of hemorrhagic MI. Left half, 90-min I/R of LAD artery consistently lead to hMI as evidenced by LGE and T2*CMR from a rat sacrificed at 24-hrs post reperfusion; and verified by histology showing extravasation of red blood cells on H&E and pale blue regions on PB stains. But regions negative for MI and hemorrhage (remote myocardium) did not show tissue damage. Right half, 30-min I/R of LAD always lead to non-hMI on T2* (no dark core). H&E sections showed tissue damage but not red cell extravasation and absence of PB stained regions within MI. Remote myocardia showed no evidence of tissue damage or hemorrhage. 60-min I/R lead to disproportionate hMIs (˜60% hMI and ˜40% non-hMI). Reproducibility of this model has been confirmed. Notably, MI size of rats undergoing 60-min I/R were not different at two research institutes: 32±6% (LSU) vs 35%±5% (Cedars), p=0.43.

FIGS. 11A-11H depict oxidative stress, ferroptosis biomarkers and autophagy in MIs with 90-min I/R. Oxidative stress (total ROS; FIG. 11A), superoxides (FIG. 11B), ferroptosis (NOX gene; FIG. 11D) and protein expression (FIG. 11H) were upregulated in the pen-infarct zone in the acute phase compared to sham control with decreasing antioxidant level over a 4-week period following reperfusion. Key autophagy genes, BCL-2 (FIG. 11E), LC3 (FIG. 11F), and ATG5 (FIG. 11G) were also upregulated and remained elevated at 4 weeks. Since late reperfusion leads to hMI, the upregulation of these markers may be instigated by the evolving forms of iron from hMI.

FIGS. 12A and 12B depict the therapeutic benefits of hemopexin (Hx). FIG. 12A shows that within MI, total ROS production was significantly lower in Hx treated group compared to PBS treated group at 24 hrs post reperfusion. FIG. 12B shows that Hx group also showed better LV remodeling at 1-month post reperfusion than the PBS-treated group. Note the ventricular dilation in rats treated with PBS (not evident Hx treated rates). Long- and shor-axis (along dotted lines) views are shown.

FIG. 13 depicts a proposed mechanism of ferroptosis mediated infarct expansion in acute hemorrhagic MI.

FIG. 14 depicts that Fe³⁺ crystals drive persistent inflammation in the chronic phase of MI. Monocytes recruited from blood, differentiating into tissue macrophages transport Fe3+ through macroautophagy within lysosomes. Crystalized iron within lysosomes damage the lysosomal membrane causing the lysosomal enzymes to spill into the cytosol and damage mitochondrial membrane resulting in oxidative stress. An upregulated autophagic pathway not being able to clear the damaged mitochondria results in the accumulation of damaged mitochondria. These lead to inflammasome activation causing the macrophages to release proinflammatory cytokines, which drive adverse LV remodeling.

FIG. 15 depicts TEM, atomic-resolution imaging and X-ray EDS showing the features of chronic iron deposits in crystalline form at 8 weeks post MI. Macrophage with marked intracellular electron-dense material organized into nodules (top left, top middle). Lysosomes containing iron (top right). Atomic-resolution TEM images of a nanocrystal from a nodular cluster (lower left). EDS confirmed the strong presence of iron (lower middle). Diffraction pattern reveals an exact fit with the pattern of a 6-HFO (lower right).

FIG. 16 is a bar graph depicting the total ROS production (μM/mg protein/min) in cardiomyocyte culture treated with different agents or control, indicating the importance of physiochemical state of degradation products of hemorrhage being the therapeutic targets.

FIGS. 17A-17C depict temporal evaluation of troponin T and MI size in early phase of MI depends of hemorrhage status in ST-Elevation Myocardial Infarction (STEMI) patients. FIG. 17A shows mean troponin T (TnT) values in IMH(−) and IMH(+) patients at 12 hr, 24 hr, 72 hr and 5 days to 7 days post percutaneous coronary intervention (PCI). FIG. 17B shows mean TnT values for IMH(+) was significant higher than IMH(−) independent of MVO status at as early as less than 12 hr, and decreased systemically but remain higher than IMH(−) between 24 hr and Day 5 to 7. However, the peak of troponin T level was presented later at 24 h post PCI with a significant lower level in patients without IMH. FIG. 17C shows the MI size (percentage of left ventricular mass) assessed by CMR was also significant larger in patients with IMH(+) group compared with patients IMH(−).

FIGS. 18A-18C show processed late gadolinium enhancement (LGE), processed T2* and PET images of hearts with hemorrhagic MI in acute phase. FIG. 18A shows representative, highlighted pixels in processed LGE (depicting MI size) at day 0, day 1, day 3, day 5 and day 7. FIG. 18B shows T2* (depicting hemorrhage). Compared with day 0 post reperfusion, highlighted pixels in processed LGE on Day 1 expanded significantly, which was even larger than AAR defect in PET image (FIG. 18C). Color-code area in Bulls-eye plots for LGE was larger than the defect in polar map for PET as well. AAR=area at risk.

FIGS. 19A-19C show processed LGE, processed T2* and PET images of hearts with non-hemorrhagic MI in acute phase. Representative, highlighted pixels in processed LGE (depicting MI size; FIG. 19A) at day 0, day 1, day 3, day 5, day 7 and T2*(depicting hemorrhage; FIG. 19B) cardiac MRI from an animal with non-hemorrhagic MI are shown. Compared with day 0 post reperfusion, highlighted pixels in processed LGE expanded slightly on Day 1, which was much smaller than defect in PET image (FIG. 19C). Color-code area in Bulls-eye plots for LGE was also smaller than the AAR defect in polar map for PET as well.

FIGS. 20A and 20B depict the temporal evaluation of LGE-based infarct size, in the canines with hemorrhagic and non-hemorrhagic MI. Box plot showed median infarct size measured using LGE (LV %) normalized by PET AAR in the canines without hemorrhagic MI slight expanded on day 1 and slightly decreased from day 2 through day 7, and the MI size on Day 7 is as similar as Day 0 post reperfusion (FIG. 20B). However, in the canines with hemorrhagic MI, the LGE based infarct size expanded significantly on day 1 and tended to be stabilized from Day 1 through Day 7 post reperfusion (FIG. 20A), whose difference in infarct size between day 0 and day 7 is significant (p<0.001) (FIG. 20A). Compared with the canines with non-hemorrhagic MI, LGE-based infarct size is significant higher on week 8 in canines with hemorrhagic MI.

FIGS. 21A and 21B depict the differences in Expand index in early and late acute phase of MI depends between hemorrhagic status and non-hemorrhagic status, as well as the relationship between expand index and hemorrhagic volume. FIG. 21A shows rate of infarct expansion (%) on day x (Dx; compared to day 0, DO)=(MI size day x−MI size day 0)/PET AAR. The mean rate of infarct expansion is significant higher in hemorrhagic (IMH+) compared with non-hemorrhagic (IMH−) canines on day 1, day 3, day 5 and day 7. FIG. 21B is a scatter plot showing the relationship between hemorrhage volume and rate of infarct expansion as determined on day 1 vs day 0, day 3 vs day 0, and day 7 vs day 0. Results from linear regression analysis are shown in the inset legend. Lines of best fit from regression analysis between hemorrhage volume and expand index are shown for day 1.

FIGS. 22A and 22B depict the temporal evaluation of myocardial salvage, in the canines with hemorrhagic MI or non-hemorrhagic MI, respectively. Box plot showed mean myocardial salvage calculated using LGE based infarct size and PET based AAR. The mean myocardial salvage is similar between canines with and without hemorrhagic on day 0. In the canines without hemorrhage, mean myocardial salvage tends to be stable from day 0 (DO) through day 7 (D7) (FIG. 22B). However, the mean myocardial salvage abruptly decreases on day 1 and continued with very low from day 1 through day 7 for canines with hemorrhagic MI (FIG. 22A).

FIG. 23A depicts a schematic showing the time points at which [cTn] were measured and CMR was performed in STEMI patients. FIG. 23B depicts a schematic showing the time points at which [cTn] were measured and CMR was performed in STEMI patients.

FIGS. 24A-24C depict [cTn] kinetics and acute MI size in reperfused STEMI patients depend on hemorrhage status. FIG. 24A shows a box-plot of [cTn] in patients with intramyocardial hemorrhage (IMH+ (red)) and without intramyocardial hemorrhage (IMH− (blue)) at 12 hr, 24 hr, 72 hr and 5-7 days, post PCI. FIG. 24B shows that in IMH+ patients, mean [cTn] was significantly elevated compared to the IMH− patients at all time points post PCI. [cTn] peaked earlier in IMH+ patients compared to IMH− patients, independent of MVO status. FIG. 24C shows that consistent with peak [cTn] in the IMH+ and IMH− groups, MI size (% LV) as assessed by CMR on 5-9 days post PCI was larger in IMH+ patients compared with the IMH− group (p<0.001).

FIGS. 25A-25C depict [cTn] kinetics and acute MI size in canines with reperfused MI, which parallel findings in STEMI patients. FIG. 25A shows a box-plot of [cTn] in canines with intramyocardial hemorrhage (IMH+ (red)) and without intramyocardial hemorrhage (IMH− (blue)) at baseline (BL), 24 hr, 48 hr, 72 hr and 7 days post reperfusion. FIG. 25B shows in IMH+ animals, mean [cTn] was significantly elevated compared to the IMH− animals at all time points post PCI. FIG. 25C shows that consistent with peak [cTn] in the IMH+ and IMH− groups, MI size (% LV) as assessed by CMR at 7 days post reperfusion was larger in IMH+ group of animals compared to the IMH− group (p<0.001). Importantly, the [cTn] and MI size behavior in IMH+ and IMH− groups in canines paralleled the observations in revascularized STEMI patients.

FIGS. 26A-26C depict that reperfusion leads to rapid and expansive myocardial damage within the area-at-risk in canines with IMH. FIG. 26A shows the area-at-risk (AAR) determined using ¹³N-ammonia PET during complete LAD occlusion. Representative apical, mid, and basal slices are shown, along with polar map identifying the “bloodshed” region corresponding to the AAR are shown. FIG. 26C shows representative unprocessed (raw, top row) late gadolinium enhancement (LGE, top) and processed LGE, which identify MI territory (processed, second row), at <1 hr, 24 hrs, 72 hrs, 5 and 7 days post reperfusion. In the processed images suspected MVO is shaded brown, infarcted region is shaded yellow, epicardial and endocardial borders are represented as green and red contours, respectively. Third and fourth rows of FIG. 26B shows fraction of the myocardium infarcted at a segmental level and polar plots of infarct transmurality, respectively. FIG. 26B shows evidence of IMH within the MI zone based on T2* CMR (72-hrs post reperfusion) in the basal, mid and apical short-axis views, and a segmental representation on the right. Compared to MI size within 1-hr of reperfusion, MI size at 24 hrs is substantially larger and by day 7 encompasses most of the area-at-risk.

FIGS. 27A-27C depict reperfusion results in mild increase in myocardial damage within the area-at-risk in canines without IMH. FIG. 27A shows the area-at-risk (AAR) determined using ¹³N-ammonia PET during complete LAD occlusion. Representative apical, mid, and basal slices are shown, along with polar map identifying the “bloodshed” region corresponding to the AAR are shown. FIG. 27C depicts representative unprocessed (raw, top row) late gadolinium enhancement (LGE, top) and processed LGE, which identify MI territory (processed, second row), at <1 hr, 24 hrs, 72 hrs, 5 and 7 days post reperfusion. In the processed images suspected MVO is shaded brown, infarcted region is shaded yellow, epicardial and endocardial borders are represented as green and red contours, respectively. Third and fourth rows of FIG. 27C shows fraction of the myocardium infarcted at a segmental level and polar plots of infarct transmurality, respectively. FIG. 27B shows absence of IMH within the MI zone based on T2* CMR (72-hrs post reperfusion) in the basal, mid and apical short-axis views, and a segmental representation on the right. Compared to MI size within 1-hr of reperfusion, there is no substantial changes in MI size by day 7 encompasses within the area-at-risk.

FIGS. 28A-28D depict that the temporal evolution of MI size normalized to area-at-risk is different between MIs with and without hemorrhage. Box plot of infarct volume normalized to AAR in canines with and without hemorrhagic MI within 1-hr of reperfusion to day 7 post reperfusion are shown in FIG. 28A and FIG. 28B, respectively. As replotted in FIG. 28C, in canines with hemorrhagic MI, infarct volume normalized to AAR expanded significantly by 24 hrs and then stabilized through 7 days post reperfusion; and in contrast, non-hemorrhagic MI showed only a mild increase in MI volume normalized to AAR by 24 hrs and then stabilized through 7 days post reperfusion.

FIG. 28D shows that the scar volumes (% LV) at week 8 post reperfusion were consistent with observations at day 7 post reperfusion, evidening significantly a larger scar size in infarcts with IMH compared to MIs without IMH in the acute phase, despite that both having similar AAR.

FIGS. 29A-29D depict that the temporal evolution of infarct transmurality is different between MIs with and without hemorrhage. Box plot of MI transmurality in canines with and without hemorrhagic MI within 1-hr of reperfusion to day 7 post reperfusion are shown in FIG. 29A and FIG. 29B, respectively. As replotted in FIG. 29C, in canines with hemorrhagic MI, infarct transmurality increased significantly by 24 hrs and then stabilized through 7 days post reperfusion; and in contrast, non-hemorrhagic MI showed only a mild increase in MI transmurality and then stabilized through 7 days post reperfusion. FIG. 29D shows that MI transmurality at week 8 post reperfusion were consistent with observations at day 7 post reperfusion, evidencing a significantly larger MI transmurality in infarcts with IMH compared to MIs without IMH in the acute phase, despite having similar AAR.

FIGS. 30A-30D depict that the rate of MI expansion following reperfusion is sensitive to time after reperfusion and hemorrhage volume. FIG. 30A shows a boxplot of the rate at which MI expands in the days following reperfusion. FIG. 30B shows the scatter plot between the rate of MI expansion and hemorrhage volume at key time intervals following reperfusion. FIG. 30C shows a boxplot of the rate at which transmurality of the MI changes in the days following reperfusion. FIG. 30D shows the scatter plot between the rate of change of infarct transmurality and hemorrhage volume at key time intervals following reperfusion. Results from linear regression analysis for 30B and 30D are also shown within the respective panels. Both the change in infarct size and transmural extension of MI is the greatest within the first 24-hrs of reperfusion and is highly dependent on the extent of intramyocardial hemorrhage. D0-D1 denotes Day 0-Day 1; D1-D2 denotes Day 1-Day 2; D2-D3 denotes Day 2-Day 3; and D3-D5 denotes Day 3-Day 5 post reperfusion.

FIGS. 31A and 31B depict that the extent of myocardial salvage post reperfusion is significantly diminished in canines with reperfusion hemorrhage. 31A: Box plot shows the temporal changes in myocardial salvage following reperfusion in animals with reperfusion hemorrhage. 31B: Box plot shows the temporal changes in myocardial salvage following reperfusion in animals without reperfusion hemorrhage. Note the marked decrease in myocardial salvage in the presence of reperfusion hemorrhage compared to those without reperfusion hemorrhage.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, N.Y. 2006); March, Advanced Organic Chemistry Reactions, Mechanisms and Structure 7^{th} ed., J. Wiley & Sons (New York, N.Y. 2013); and Sambrook and Russel, Molecular Cloning: A Laboratory Manual 4^{th} ed., Cold Spring Harbor Laboratory Press (Cold Spring Harbor, N.Y. 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The terms “treat,” “treatment,” “treating,” or “amelioration” when used in reference to a disease, disorder or medical condition, refer to both therapeutic treatment and prophylactic or preventative measures, wherein the object is to prevent, reverse, alleviate, ameliorate, inhibit, lessen, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a disease, disorder or medical condition is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results, or lower the chances of the individual developing the condition even if the treatment is ultimately unsuccessful. Those in need of treatment include those already with the condition as well as those prone to have the condition or those in whom the condition is to be prevented.

“Beneficial results” or “desired results” may include, but are in no way limited to, lessening or alleviating the severity of the disease condition, preventing the disease condition from worsening, curing the disease condition, preventing the disease condition from developing, lowering the chances of a patient developing the disease condition, decreasing morbidity and mortality, and prolonging a patient's life or life expectancy. As non-limiting examples, “beneficial results” or “desired results” may be alleviation of one or more symptom(s), diminishment of extent of the deficit, stabilized (i.e., not worsening) state of infarct area size, infarct myocardium wall thickness, fat deposition and remodeling in infarction, oxidative stress in the infarct, and/or amelioration or palliation of symptoms associated with myocardial infarction or intramyocardial hemorrhage with infarction.

“Diseases”, “conditions” and “disease conditions,” as used herein may include, but are in no way limited to any form of cardiovascular conditions, diseases or disorders. Cardiovascular diseases are a class of diseases that involve the heart or blood vessels. Non-limiting examples of cardiovascular disease include: myocardial infarction, acute myocardial infarction, hemorrhagic myocardial infarction, persistent microvascular obstruction (PMO), microvascular obstruction (MO), ischemic heart disease (IHD), coronary artery disease, coronary heart disease, cardiomyopathy, stroke, hypertensive heart disease, heart failure, pulmonary heart disease, ischemic syndrome, coronary microvascular disease, cardiac dysrhythmias, rheumatic heart disease (RHD), aortic aneurysms, cardiomyopathy, atrial fibrillation, congenital heart disease, endocarditis, inflammatory heart disease, endocarditis, inflammatory cardiomegaly, myocarditis, valvular heart disease, cerebrovascular disease, and peripheral artery disease (PAD).

The term “administering,” refers to the placement an agent as disclosed herein into a subject by a method or route which results in at least partial localization of the agents at a desired site. “Route of administration” may refer to any administration pathway known in the art, including but not limited to aerosol, nasal, via inhalation, oral, anal, intra-anal, peri-anal, transmucosal, transdermal, parenteral, enteral, topical or local. “Parenteral” refers to a route of administration that is generally associated with injection, including intratumoral, intracranial, intraventricular, intrathecal, epidural, intradural, intraorbital, infusion, intracapsular, intracardiac, intradermal, intramuscular, intraperitoneal, intrapulmonary, intraspinal, intrasternal, intrathecal, intrauterine, intravascular, intravenous, intraarterial, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the pharmaceutical compositions can be in the form of aerosol, lotion, cream, gel, ointment, suspensions, solutions or emulsions. In accordance with the present invention, “administering” can be self-administering. For example, it is considered as “administering” that a subject consumes a composition as disclosed herein.

A “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In addition, the methods described herein can be used to treat domesticated animals and/or pets. “Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

A “subject in need” of diagnosis or treatment for a particular condition can be a subject suspected of having that condition, diagnosed as having that condition, already treated or being treated for that condition, not treated for that condition, or at risk of developing that condition. In some embodiments, the subject in need is a subject with an ST elevation myocardial infarction (STEMI). In some embodiments, the subject in need is a subject with signs of myocardial infarction, such as chest pain, shortness of breath. In some embodiments, the subject in need is a subject experiencing or having undergone reperfusion following myocardial infarction. In some embodiments, the subject in need is a subject suffering from or having a high risk of having hemorrhage following intervention (e.g., reperfusion) after myocardial infarction.

“Hemorrhage” as used herein refers to pooling of blood within a vessel or extravasation of blood into the interstitial space.

The process of “lipomatous metaplasia” (LM) refers to a condition where the collagen within chronic scars is replaced by metaplastic adipose tissue as part of the healing cascade after MI.

“Troponins” (or troponin) are a group of proteins found in skeletal and heart (cardiac) muscle fibers that regulate muscular contraction. Troponin tests measure the level of cardiac-specific troponin in the blood to help detect heart injury. Normally, troponin is present in very small to undetectable quantities in the blood. When there is damage to heart muscle cells, troponin is released into the blood. The more damage there is, the greater the concentration in the blood. There are three types of troponin proteins: troponin C, troponin T, and troponin I. Troponin C initiates contraction by binding calcium and moves troponin I so that the two proteins that pull the muscle fiber shorter can interact. Troponin T anchors the troponin complex to the muscle fiber structure. There is little or no difference in troponin C between skeletal and cardiac muscle, but the forms of troponin I and troponin T are different. Measuring the amount of cardiac-specific troponin T or troponin I in the blood can help identify individuals who have experienced damage to their heart. In some embodiments, as shown in the Examples below, measuring a “cardiac troponin” level refers to measuring cardiac troponin I in animals, and measuring cardiac troponin T in human subjects/patients. In other embodiments, measuring a troponin level comprises measuring a level of troponin T, a level of troponin I, a level of troponin C, or a combination thereof.

Non-limiting symptoms of myocardial infarction include pressure or tightness in the chest; pain the in the chest, back, jaw and other upper body areas that lasts more than a few minutes or that goes away and comes back; shortness of breath; sweating; nausea; vomiting; anxiety; a cough; and lightheadedness or sudden dizziness.

Generally acute phase of myocardial infarction refers to a period of time from the onset of coronary obstruction or symptoms of acute myocardial infarction, including amplification of reactive oxygen species activity from excess heme in the infarct zone, to the transition of ferrous to ferric iron. In some aspects, the acute phase includes the first few hours from onset of symptoms, the first few days (typically 1-3 days), and in some instances into about 1 week from onset of myocardial infarction. In some aspects, the acute phase is identified by determining a dominance of ferrous iron, Fe(II), e.g., mostly in the form of heme, and optionally coupled by evidence of lack of ferric iron crystals in or near the infarcted myocardium. In some aspects, the infarct area expands in the acute phase (e.g., 48-72 hours since coronary obstruction) of myocardial infarction. In some embodimnts, “about” 1 week refers to between 5 days and 9 days. In some embodiments, “about” 1 week refers to between 6 and 8 days. In some embodiments, “about” 1 week refers to between 5 and 8 days. In some embodiments, “about” 1 week refers to between 6 and 9 days.

In further aspects, sub-acute phase is beyond the acute phase, and before the chronic phase, and the sub-acute phase is from days to weeks after MI. Acute phase can be hours to days after MI. Chronic phase can be weeks to months after MI.

Generally, chronic phase of myocardial infarction refers to a period of weeks or months since the coronary obstruction or onset of infarction symptoms where persistent pro-inflammatory or inflammatory burden are exerted. In some aspects, the chronic phase includes 7 days and onward since the onset of infarction symptoms, e.g., 4 weeks, 8 weeks, 3 months, or 6 months.

Generally, iron chelators include an intracellular iron chelator, extracellular iron chelator, or combination thereof. The intracellular iron chelator may chelate ferrous iron, ferric iron, and combinations thereof. The extracellular iron chelator may chelate ferrous iron, ferric iron, and combinations thereof.

Treatments or Intervention

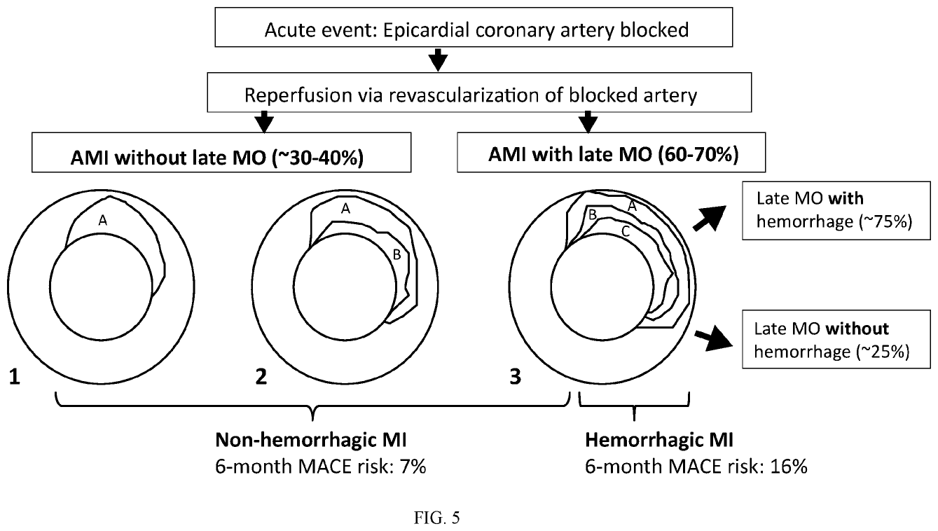

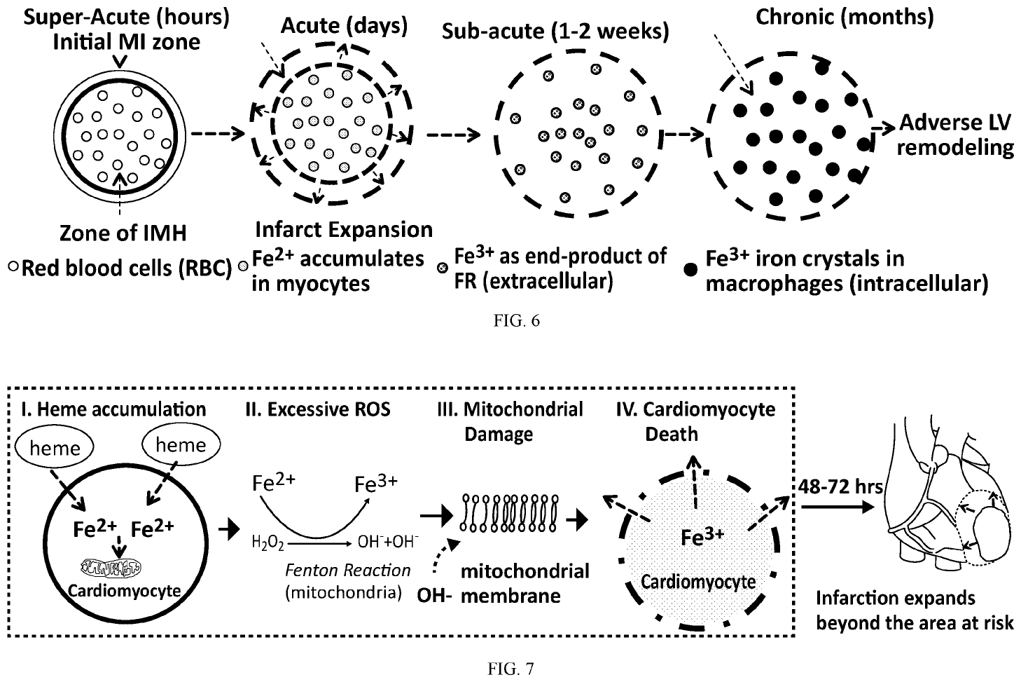

Various types of MI have been proposed based on the “wave front hypothesis.” More than 60% of acute MIs have late microvascular obstruction (type 3 in FIG. 5), resulting in “no-reflow” (zone C in FIG. 5), despite re-establishing flow in the culprit artery. Late microvascular obstruction (late MO) has emerged as a key risk factor for major adverse cardiac events (MACEs). Hemorrhage is present in ˜75% of MIs with late MO, conferring >50% increased risk for MACE over MIs with late MO but no hemorrhage. Thus, hemorrhagic MIs (hMI) present the greatest risk for MACE among all MI patients. In the acute phase, reperfusion injury can increase infarct size; and large MIs often have hemorrhage. hMIs are significantly larger than non-reperfused MIs, indicating that hemorrhage mediates MIs to expand beyond the area at risk. Moreover, late MO limits the influx of inflammatory cells into the MI and thereby delays infarct healing in the sub-acute phase.

Various embodiments of the invention provide therapeutic treatment or intervention for myocardial infarction, heart failure in relation to heart muscle conditions, and heart remodeling from heart muscle related conditions. In some embodiments, the therapeutic treatment or intervention methods disclosed herein involves administering one or more iron chelators, anti-inflammatory agents (e.g., colchicine), etc. to treat myocardial or coronary infarction, heart failure in relation to heart muscle malfunction (e.g., cardiomyopathy), and/or improve heart remodeling resulting from heart muscle malfunction. In some embodiments, the methods are not intended for treating other cardiovascular diseases such as atherosclerosis. In some embodiments, the therapeutic treatment or intervention methods disclosed herein are directed to a subject with treat myocardial or coronary infarction, or heart failure in relation to heart muscle malfunction, who does not have atherosclerosis.

Acute Phase

Without wishing to be bound by a theory, the large quantity of heme from red blood cells in the early phase of hMI promotes ferroptosis of surviving myocytes in the border zone of MI, resulting in ferric iron as a byproduct and leading to infarct expansion. An early degradation byproduct of hemorrhage is heme, which when internalized is further broken down into ferrous (Fe²⁺) irons. When excessive heme is present in the extracellular space, it is taken up by cardiomyocytes. The resulting intracellular Fe²⁺ amplifies Fenton reactions to produce ROS. Excessive ROS exhaust the anti-oxidative capacity of cells, destabilize the mitochondria and promote cell death. Subsequently, in the sub-acute to chronic phases of MI, iron within MI is in ferric state and when it is internalized within lysosomes of macrophages, it crystallizes; and macrophages attempt to clear the iron in the MI zone by internalizing it for degradation within lysosomes. This process leads to accumulation of crystalline iron in lysosomes, resulting in in lysosomal leakage, secondary mitochondrial dysfunction, and further oxidative stress; and upregulation of autophagy with impaired flux (due to lysosomal damage), thereby limiting clearance of damaged mitochondria. Both of these processes invoke inflammasome activation and release of proinflammatory cytokines from the macrophages, and thereby driving adverse remodeling. Reperfused MIs with hemorrhage are significantly larger than non-reperfused MIs (including MIs without hemorrhage). hMIs lead to persistent iron deposition within MI; new macrophages are recruited to the site of iron; and iron within MI is an independent risk factor for adverse remodeling in the chronic period in animals and patients—providing a strong correlation among hMI, iron deposition, inflammation, and adverse remodeling. These findings support the notion that hemorrhage may be a key driver of myocardial damage in reperfused MIs. Lipomatous metaplasia (LM), a process where collagen within chronic scars is replaced by metaplastic adipose tissue, of infarcted myocardium is driven by perpetual iron-induced macrophage activation, lipid oxidation, foam cell formation, ceroid production, foam cell apoptosis and iron recycling, in a process unique to hemorrhagic MIs that culminates in adverse anatomic and functional remodeling; and some aspects of the invention include that these adverse effects can be mitigated through timely reduction of iron from the hemorrhagic MI zone.

The evolving changes of hemorrhage within the infarct zone precipitate a heme-iron mediated death (ferroptosis) of surviving myocytes in the acute MI territory: and the formation of ferric-iron crystals within macrophages that attempt to remove them, which polarizes the macrophages to a proinflammatory state in chronic MI. Hemorrhage promotes a chain of time-dependent events, which damage the heart through infarct expansion in acute phase and adverse left ventricle remodeling in chronic phase of MI. In one aspect, hemorrhage causes myocyte death through heme-mediated ferroptosis in the acute MI zone; and upregulates autophagy, lysosomal leakage and mitochondrial damage, which promotes inflammasome activation favoring a proinflammatory macrophage phenotype in chronic MI zone. The location [extracellular vs. intracellular (myocyte vs. macrophage)], oxidation state (Fe²⁺ vs. Fe³⁺), and form (free vs. crystalline) of iron following reperfusion is time dependent.

Further aspects of the invention include an improved therapy that accounts for the temporal byproducts of hemorrhage and targets the byproducts accordingly, so as to decrease infarct expansion in acute phase and forestall adverse remodeling in chronic phase of hMI, thereby protecting the hearts from infarct expansion and rapid adverse remodeling, especially for patients having symptoms or showing signs of (intramyocardial) hemorrhagic myocardial infarctions. Chelation therapy has been tried for MI in prior studies, but it has been unsuccessful as it did not target hMI or appropriate iron derivatives of heme at the right time.

Exemplary iron chelators and their specific iron target are shown in Table 1 for administration in an effective amount to a subject showing symptoms or having been diagnosed with myocardial infarction so as to bind, occupy or inactivate iron content from the myocardial infarction region. Previous studies with divalent cation chelators have not proven to be effective in reducing MACE. Recent trial to assess chelation therapy (TACT) in post MI patients showed that 6 months of ethylenediaminetetraacetic acid (EDTA) therapy starting 6-weeks post MI did not decrease MACE. Notably, EDTA is not specific (or dosed) for ferric iron; cannot cross cell membranes; and ferric-EDTA complex is unstable: it can be transformed to ferrous-EDTA in vivo and participate in Fenton chemistry and enable the production of ROS. Applicant's data indicate in various embodiments, at 6-weeks post MI, iron within MI is intracellular and is trivalent; and EDTA treatment in post MI rats did not decrease adverse LV remodeling. Therefore, in some aspects, the prevention and/or intervention methods against infarct expansion does not include administering EDTA to a subject showing symptoms or having been diagnosed with myocardial infarction. In other aspects, prevention and/or intervention methods are provided for a subject showing symptoms or having been diagnosed with myocardial infarction, wherein no EDTA is administered. In some embodiments, a cocktail of iron chelators includes (1) BPD and DXZ, (2) BPD and DFP, (3) BPD and DFX, (4) BPD and DFO, (5) DXZ and DFP, (6) DXZ and DFX, (7) DXZ and DFO, (8) DFP and DFX, (9) DFP and DFO, (10) DFX and DFO, (11) BPD, DXZ, and DFP, (12) BPD, DXZ, and DFX, (13) BPD, DXZ, and DFO, (14) BPD, DFP, and DFX, (15) BPD, DFP, and DFO, (16) BPD, DFX and DFO, (17) DXZ, DFP, and DFX, (18) DXZ, DFP, and DFO, (19) DXZ, DFX, and DFO, (20) DFP, DFX, and DFO, (21) BPD, DXZ, DFP, and DFX, (22) BPD, DXZ, DFP, and DFO, (23) BPD, DXA, DFX, and DFO, (24) BPD, DFP, DFX, and DFO, (25) DXZ, DFP, DFX, and DFO, or (26) BPD, DXZ, DFP, DFX, and DFO, or (27) any of the above in combination with EDTA, or (28) EDTA with one, two, three, four or all five of DFO, BPD, DXZ, DFP, and DFX.

TABLE 1
Exemplary iron chelators singly or in combination
as a cocktail for treatment and their properties.

| | Oxidation state of | Location | Molecular |
|---|---|---|---|
| Chelator | chelated iron and | EC: extracellular | weight |
| (acronym) | chemical stability | IC: intracellular | (g/mol) |
| disodium salt | Unstable complex | EC | 292 |
| (EDTA) | with Fe²⁺ and Fe³⁺ | (lipid insoluble) | |
| deferoxamine | Stable Fe³⁺ | EC | |
| (DFO) | complex | (lipid insoluble) | 560 |
| 2,2-bipyridl | Stable Fe²⁺ | IC & EC | 156 |
| (BPD) | complex | | |
| Dexrazoxane | Stable Fe²⁺ | IC & EC | 268 |
| (DXZ) | complex | | |
| Deferiprone | Stable Fe³⁺ | IC & EC | 139 |
| (DFP) | complex | | |
| Deferasirox | Stable Fe³⁺ | IC & EC | 373 |
| (DFX) | complex | | |

Methods for preventing or reducing the likelihood of infarct expansion, improving the likelihood of cardiac remodeling, and/or reducing the likelihood of adverse effect of reperfusion, in a subject in need thereof include administering an effective amount of an agent that binds heme, a scavenger of heme, an agent that regulates heme, or a ferrous iron chelator to the subject during the acute phase (e.g., within 72 hours after the first sign or symptom of myocardial infarction), wherein the subject in need thereof shows symptoms, has experienced or has been diagnosed with myocardial infarction. In some aspects of the methods, ferric iron chelator is not administered during the acute phase. In various embodiments, the subject in need thereof is a subject with hemorrhagic myocardial infarction. In some embodiments, the subject in one or more of the methods is one with reperfusion injury, or intramyocardial hemorrhage following reperfusion. In further embodiments, the subject in need there of in one or more of the methods is a mammalian with an increased blood level of troponin within 12-24 hours following reperfusion therapy, compared to a baseline level (e.g., a baseline level is one of the same subject obtained prior to the reperfusion therapy such as PCI; or a baseline level is one obtained within 12 hours prior to reperfusion; or a baseline level is one obtained subsequent to onset of MI symptoms but within 12 hours prior to reperfusion).

Current standard of care (SOC) does not discriminate between hemorrhagic and non-hemorrhagic patients; hence they are not managed differently. We provide methods to retrospectively identify a hemorrhagic infarction, as well as methods to identify an ongoing hemorrhagic infarction, based on repeat blood sampling and measuring temporal profiles of the troponin level.

In various embodiments, the subject in need thereof is a subject identified to be hemorrhagic following myocardial revascularization (e.g., percutaneous coronary intervention, PCI), whose troponin level peaks within 18 hours following PCI, e.g., peaks (or continues rising) at 8 hours, 9 hours, 10 hours, 11 hours, 12 hours, 13 hours, 14 hours, or 15 hours following the PCI—and the troponin level decreases after 18 hours following the PCI, and whose troponin level has a difference between the highest obtained value and a baseline value (e.g., measured pre-PCI) that is 1.5 ng/mL or greater. In some embodiments, the difference is 1.5-3 ng/mL. In some embodiments, the difference is 3-4 ng/mL. In some embodiments, the difference is 4-5 ng/mL. In some embodiments, the difference is 5-6 ng/mL. In some embodiments, the difference is 6-7 ng/mL. In some embodiments, the difference is 7-8 ng/mL. In some embodiments, the difference is 8-9 ng/mL. In some embodiments, the difference is 9-10 ng/mL. In some embodiments, the difference is 10-12 ng/mL. In some embodiments, the difference is 12-15 ng/mL, or greater. And in some embodiments, the rate is a combination of any two or more of those listed above in this paragraph. In some embodiments, the difference is at least 6 ng/mL. In some embodiments, the difference is at least 7 ng/mL. In some embodiments, the difference is at least 8 ng/mL. In some embodiments, the difference is at least 9 ng/mL.

In other embodiments, the subject is one identified to be non-hemorrhagic following myocardial revascularization (e.g., PCI), whose troponin level peaks or continues to rise after 18 hours post PCI, e.g., continues rising about 20 hours, 21 hours, 22 hours, 23 hours, 24 hours, or 25 hours following the PCI, and subsequently followed by a decrease, and whose troponin level has a difference between the highest obtained value and a baseline value (e.g., measured pre-PCI) that is less than 1.5 ng/mL. In some embodiments, the difference is a difference of 0.1-0.5 ng/mL. In some embodiments, the difference is 0.5-1 ng/mL. In some embodiments, the difference is 1-1.4 ng/mL.

In further embodiments, the subject in need thereof is one identified as having an active/on-going hemorrhagic MI, whose troponin level within the first 12 hours following revascularization (e.g., PCI) is rising at a rate greater than 0.2 ng/mL/hr. In some embodiments, the rate is 0.2-0.3 ng/mL/hr. In some embodiments, the rate is 0.3-0.4 ng/mL/hr. In some embodiments, the rate is 0.4-0.5 ng/mL/hr. In some embodiments, the rate is 0.5-0.6 ng/mL/hr. In some embodiments, the rate is 0.6-0.7 ng/mL/hr. In some embodiments, the rate is 0.7-0.8 ng/mL/hr. In some embodiments, the rate is 0.8-0.9 ng/mL/hr. In some embodiments, the rate is 0.9-1.0 ng/mL/hr. In some embodiments, the rate is 1.0-1.2 ng/mL/hr. In some embodiments, the rate is 1.2-1.5 ng/mL/hr. And in some embodiments, the rate is a combination of any two or more of those listed above in this paragraph. In some embodiments, the subject identified with active hemorrhagic MI has a troponin level that rises within 12 hours following revascularization at a rate of 0.4 ng/mL/hr or greater. In some embodiments, the subject identified with active hemorrhagic MI has a troponin level that rises within 12 hours following revascularization at a rate of 0.7-1.2 ng/mL/hr. This is in comparison to non-hemorrhagic subjects whose troponin level rises at most at a pace of 0.1-0.2 ng/mL/hr in the first 12 hours following the revascularization.

In one aspect, the subject has not had reperfusion, or the administration of the agent that binds heme, the scavenger of heme, the agent that regulates heme, or the ferrous iron chelator is before performing reperfusion to the subject, in the methods above for preventing or reducing the likelihood of infarct expansion, improving the likelihood of cardiac remodeling, and/or reducing the likelihood of adverse effect of reperfusion; thereby the administration as a pre-treatment. In some aspects of the methods, ferric iron chelator is not administered during the acute phase.

In another aspect, the subject has had reperfusion attempted to restore blood flow in the heart, or the administration of the agent that binds heme, the scavenger of heme, the agent that regulates heme, or the ferrous iron chelator is after performing reperfusion to the subject, in the methods above for preventing or reducing the likelihood of infarct expansion, improving the likelihood of cardiac remodeling, and/or reducing the likelihood or severity of adverse effect of reperfusion.

In a further aspect, the subject has reperfusion-induced hemorrhage with myocardial infarction, and the administration of the agent that binds heme, the scavenger of heme, the agent that regulates heme, or the ferrous iron chelator is immediately after hemorrhagic myocardial infarction.

In another aspect, the subject has hemorrhage with myocardial infarction, wherein the subject has taken or been administered with agents that increase capillary permeability such as collagenase or vascular endothelial growth factor; therefore not necessarily related to reperfusion.

In another aspect, the subject does not show evidence of having ferric iron crystal in the infarct myocardium; or the method further include conducting cardiac imaging, and no ferric iron crystal is identified in the infarct myocardium in the subject, and administering the agent that binds heme, the scavenger of heme, the agent that regulates heme, or the ferrous iron chelator.

In yet another aspect, the subject has not been administered EDTA before or after the symptom or onset of myocardial infarction, or the methods above for preventing or reducing the likelihood of infarct expansion, improving the likelihood of cardiac remodeling, and/or reducing the likelihood or severity of adverse effect of reperfusion do not include administering EDTA to the subject.

In one embodiment, a method of preventing or reducing the likelihood of infarct expansion, improving the likelihood of cardiac remodeling, and/or reducing the likelihood of adverse effect of reperfusion in a subject showing symptoms, having experienced or having been diagnosed with myocardial infarction includes administering an effective amount of a pharmaceutical composition including 2,2-bipyridl, dexrazoxane, hemopexin, heme oxygenase (e.g., heme oxygenase 1 (HO-1)), hinokitiol, haptoglobin, albumin, ferritin, α1-microglobulin, α1-antitrypsin, glutathione-S-transferase, liver fatty acid binding protein, heme-binding protein 23, p22 heme binding protein, glyceraldehyde-3-phosphate dehydrogenase, and nuclear factor E2 related factor 2 (Nrf2), or a combination thereof, at the acute phase or within 72 hours of a symptom or from the onset of myocardial infarction to a subject in need thereof. In further embodiments, the methods of preventing or reducing the likelihood of infarct expansion, improving the likelihood of cardiac remodeling, and/or reducing the likelihood of adverse effect of reperfusion in a subject showing symptoms, having experienced or having been diagnosed with myocardial infarction further include administering an agent that regulates heme by increasing the amount of a heme binding protein and/or a heme degrading protein, and the factors include but are not limited to feline leukemia virus subgroup C receptor 1a (FLVCR1a), FLVCR2, and ATP-binding cassette subfamily G member 2 (ABCG2).

Further embodiments of the methods of preventing or reducing the likelihood of infarct expansion, improving the likelihood of cardiac remodeling, and/or reducing the likelihood of adverse effect of reperfusion in the subject include selecting a subject showing symptoms of, having experienced or having been diagnosed with myocardial infarction, optionally further been treated with reperfusion immediately following onset or first symptom of myocardial infarction, or a subject showing symptoms of, having been diagnosed or having experienced intramyocardial hemorrhage with myocardial infarction, and administering an effective amount of a pharmaceutical composition including 2,2-bipyridl, dexrazoxane, hemopexin, heme oxygenase-1 hinokitiol, haptoglobin, albumin, ferritin, al-microglobulin, al-antitrypsin, glutathione-S-transferase, liver fatty acid binding protein, heme-binding protein 23, p22 heme binding protein, glyceraldehyde-3-phosphate dehydrogenase, Nrf2, or a combination thereof, at the acute phase or within 72 hours of a symptom or from the onset of myocardial infarction to a subject in need thereof.

Acute Phase and Chronic Phase

Methods for treating myocardial infarction, such as preventing or reducing the likelihood of infarct expansion, improving cardiac remodeling, and reducing the likelihood or severity of adverse effect associated with reperfusion after myocardial infarction, in a subject in need thereof include administering an effective amount of an agent that binds heme, a scavenger of heme, an agent that regulates heme, (e.g., heme oxygenase-1, haptoglobin, albumin, ferritin, α1-microglobulin, α1-antitrypsin, glutathione-S-transferase, liver fatty acid binding protein, heme-binding protein 23, p22 heme binding protein, glyceraldehyde-3-phosphate dehydrogenase, Nrf2), or a ferrous iron chelator (e.g., 2,2-bipyridl, dexrazoxane, hinokitiol) during acute phase of the myocardial infarction and administering an effective amount of a ferric iron chelator during the chronic phase of myocardial infarction.

In one embodiment, a method of treating myocardial infarction, such as preventing or reducing the likelihood of infarct expansion, and/or treating or reducing the severity of hemorrhagic myocardial infarction thereby reducing the severity or likelihood of adverse cardiac remodeling, includes administering an effective amount of a pharmaceutical composition including 2,2-bipyridl, dexrazoxane, hemopexin, hinokitiol, heme oxygenase-1, haptoglobin, albumin, ferritin, α1-microglobulin, α1-antitrypsin, glutathione-S-transferase, liver fatty acid binding protein, heme-binding protein 23, p22 heme binding protein, glyceraldehyde-3-phosphate dehydrogenase, Nrf2, or a combination thereof, at the acute phase or within 72 hours of a first sign or symptom of myocardial infarction, and administering an effective amount of a pharmaceutical composition including deferoxamine, deferiprone, deferasirox, hinokitiol, pyridoxal isonicotinoyl hydrazone, salicylaldehyde isonicotinoyl hydrazone, or a combination thereof, during the chronic phase, after 7 days or in some embodiments after 72 hours of a first sign or symptom of myocardial infarction to a subject in need thereof. In further aspect, the method does not include administering EDTA. In another aspect, the subject has hemorrhagic myocardial infarction. In yet another aspect, the subject has reperfusion-induced hemorrhage with myocardial infarction.

In some aspects of the methods for treating myocardial infarction, (1) an agent that binds heme, a scavenger of heme, an agent that regulates heme, or a ferrous iron chelator (e.g., 2,2-bipyridl, dexrazoxane, or a combination thereof), optionally with one or more factors that increase the amount of a heme binding protein and/or a heme degrading protein, and (2) a ferric iron chelator (e.g., deferiprone, desferrioxamine, deferasirox, hinokitiol, pyridoxal isonicotinoyl hydrazone, salicylaldehyde isonicotinoyl hydrazone, or a combination thereof) are administered sequentially, and preferably at the acute phase and at the chronic phase of myocardial infarction, respectively.

In other aspects, methods for treating myocardial infarction, such as preventing or reducing the likelihood of infarct expansion, and/or treating or reducing the severity of hemorrhagic myocardial infarction thereby reducing the severity or likelihood of adverse cardiac remodeling, include administering (1) an agent that binds heme, a scavenger of heme, an agent that regulates heme, or a ferrous iron chelator (e.g., 2,2-bipyridl, dexrazoxane, or a combination thereof), optionally with one or more factors that increase the amount of a heme binding protein and/or a heme degrading protein, and (2) a ferric iron chelator (e.g., deferiprone, desferrioxamine, deferasirox, or a combination thereof) concurrently to a subject in need thereof; preferably at the acute phase (e.g., immediately after reperfusion) or within 3 days, 7 days or 1 month of onset of myocardial infarction.

Other Therapeutic Agents

In various embodiments, the therapeutic agent is provided in a pharmaceutical composition. In various embodiments, the therapeutic agent is an iron chelating agent, anti-inflammatory agent, cellular therapies, lipid-lowering agent, carbon monoxide therapy, heme-oxygenase regulating drug, an agent capable of promoting heart blood flow, an agent capable of promoting clearance of iron with enhanced macrophage activity, a phagocytosis-enhancing agent, or an agent capable of disrupting the biosynthesis of iron oxide crystals or preventing aggregation of nanocrystals, or a combination thereof.

In various embodiments, the anti-inflammatory agent is a corticosteroid, nonsteroidal anti-inflammatory drug (NSAID), anti-IL-1beta (e.g., Anakinra), anti-TNF-α (e.g., Etanercept and Infliximab), anti-IL-6 (e.g., Tocilizumab), anti-MMP (e.g., PG-116800 and Doxycycline), macrophage modulators (e.g., phosphatidylserine-presenting liposomes), NLRP3 inflammasome inhibitors (e.g., 16673-34-0 (5-chloro-2-methoxy-N-[2-(4-sulfamoylphenyl)ethyl]benzamide)), inflammasome antagonists (e.g., P2X7 antagonist), or anti-diabetic medications (for example, insulin (e.g., Humulin, Novolin, Humalog), metformin (e.g., Glucophage, Glucophage XR, Fortamet, Glumetza, Riomet), sulfonylureas, meglitinides, incretin mimetics, biguanides, amylinomimetic agent (e.g., Pramlintide), lipase inhibitors such as orlistat (e.g., Xenical, Alli), thiazolidinediones, Pioglitazone (e.g., Actos), Rosiglitazone (e.g., Avandia), corticosteroids such as Prednisone (e.g., Rayos), dipeptidyl peptidase-4 inhibitors, SGLT2 inhibitors, and glucagon-like peptide-1 analogs or agonists such as Exenatide (e.g., Bydureon, Byetta) and Liraglutide (e.g., Victoza)), or a combination thereof.

In various embodiments, the lipid-lowering agent is a statin, cholesterol absorption inhibitors (e.g., ezetimbie), bile-acid-binding resins/sequestrants (e.g., Cholestyramine), niacin, or vitamin B3, or a combination thereof.

In various embodiments, the agent capable of promoting heart blood flow is arterial CO2, adenosine, regadenoson, dypridamole, persantine, or nitric oxide, or a combination thereof. Dosage Regimen

Exemplary dosages of an agent that binds heme, a scavenger of heme, an agent that regulates heme, a ferrous iron chelator, or of a ferric iron chelator, per unit weight of a subject in the methods above include 10-100 μg, 100-200 μg, 200-300 μg, 300-400 μg, 400-500 μg, 500-600 μg, 600-700 μg, 700-800 μg, 800-900 μg, 1-5 mg, 5-10 mg, 10-20 mg, 20-30 mg, 30-40 mg, 40-50 mg, 50-60 mg, 60-70 mg, 70-80 mg, 80-90 mg, 90-100 mg, 100-200 mg, 200-300 mg, 300-400 mg, 400 mg-500 mg, 500 mg-1 g, or 1 g-10 g. Unit weight of a subject can be per kg of body weight or per subject.

Exemplary administration regimen for an agent that binds heme, a scavenger of heme, an agent that regulates heme, a ferrous iron chelator or a ferric iron chelator include immediately following onset of myocardial infarction; immediately following reperfusion; immediately following hemorrhage after reperfusion; daily for 1, 2, 3, 4, 5, 6, 7 or more days post myocardial infarction or post reperfusion; once, twice, three times or more per week for 1, 2, 3, 4, 5, 6, 7, 8 or more weeks; once, twice, three times or more per month for 1, 2, 3, 4, 5, 6, 7, 8, 9, 10, 11, 12 or more months; or a combination thereof.

In some embodiments, the administration is via oral route, intravenous route, or intracoronary route. In some embodiments, the ferrous iron chelator, the agent that binds heme or the agent that regulates heme is incorporated (e.g., coated on surfaces, encapsulated or chemically bonded to the material of the stent, or configured to be able to release therefrom) in a stent configured to recanalize occluded coronary artery of the subject.

Chronic Phase

Collectively the findings here indicate the causal connections between reperfusion hemorrhage, LM, and structural and functional remodeling of hearts sustaining hemorrhagic MIs. Notably, although the heart attempts to restore functional capacity in the early chronic phase of MI, in the absence of therapy to reduce iron and hence LM, the compensatory effort of the heart is compromised in the late chronic phase of MI. Modulating the fat content within the MI zone with an intracellular iron chelator significantly alters the functional recovery of the heart over the chronic phase of MI and prevent the establishment of heart failure.

A growing body of evidence indicates that hemorrhagic MIs lead to chronic iron deposition, and that such deposits facilitate perpetual recruitment of macrophages throughout the chronic phase of MI. Current evidence also indicates that the functional phagocytic capacity of macrophages recruited to the site of MI with abnormal iron content is significantly compromised. The present study investigated the compositional dynamics of the infarct zone with the specific goal of ascertaining differences in fat infiltration between hemorrhagic and non-hemorrhagic MI using a clinically relevant large animal model of reperfused hemorrhagic infarction. First, serial CMR showed that the extent of fat infiltration in hemorrhagic MI is directly dependent on the extent of iron in the sub-acute phase of MI. Subsequently, the present demonstrated that hemorrhagic MI territories are intimately involved in iron-induced macrophage activation, lipid oxidation, foam cell formation, ceroid production, foam cell apoptosis and iron recycling—a vicious cycle that is not observed in non-hemorrhagic MIs. Finally, the present study showed that timely reduction of iron within the hemorrhagic infarction zone is possible using a FDA-approved intracellular ferric iron chelator, and that such a therapy can decrease LM within MI zones and direct the heart towards positive anatomical and functional recovery in the post MI period.

The present study used serial in vivo CMR to determine the time-dependent relationship acute iron content within MI and the extent of fat infiltration. While no relationship between iron and fat was observed within the MI in the acute phase of MI, the relationship became stronger with the passage of time, reaching a strong correlation (R²>0.9) at 6 months post MI. Further, while the iron content within the hemorrhagic MI zones remained unchanged over the 6-month study period, treatment with DFP up to 8 weeks post MI significantly decreased the iron within the MI zone by the end of the treatment, albeit remaining unchanged thereafter to month 6. In conjunction with a reduction in iron content within the MI zone, the fat content also decreased precipitously compared to the untreated control group at the end of the treatment period. However, once the DFP treatment was halted, the fat content between week 8 and month 6 increased, although to a markedly smaller extent than the untreated control group during the same period.

In some embodiments, the present studies employed a moderate dose of DFP to demonstrate effects on chronic MI by probing the relationship between hemorrhagic MI and fat infiltration using a modest clinical dose of DFP for a limited duration. The observational and interventional CMR studies provide evidence to indicate the casual relationship between iron from hemorrhagic MI and fat infiltration within MI zone. These findings from serial in-vivo imaging augments the support formed from at a finer scale from histological investigations in hearts harvested at week 8 and month 6 following reperfused MIs.

The studies also demonstrate that macrophage population in hemorrhagic infarcts also fail to switch from a proinflammatory M1 state to an anti-inflammatory M2 state to efficiently promote myocardial scar healing. Similar to observations in chronic venous leg ulcers, the data indicates that macrophages with an unrestrained proinflammatory M1 activation state in hemorrhagic MI exhibit high expression of M2 iron scavenger receptor CD163. Given that this CD163+M1 population exclusively colocalizes with iron, extracellular lipids, apoptotic siderophage-derived foam cells and extracellular ceroid, it appears that iron-containing ceroid acts as potent proinflammatory chemoattractant promoting a self-perpetuating and amplifying loop of macrophage ingress and expansion of death zone of macrophages infiltrating the chronic MI zone.

Previous studies have shown that the scavenger receptor CD36 plays a key role in facilitating the macrophage binding and internalization of oxLDL. Specifically, oxLDL via CD36 inhibits macrophage migration, which acts as a macrophage-trapping mechanism in atherosclerotic lesions. The internalized oxLDL is known to upregulate the expression of CD36, which is known as an ‘eat me signal.’ This in turn facilitates continuous uptake of oxLDL. Activated macrophages are known to secrete various mediators which oxidize local LDL and thereby increase the pool of available oxLDL. The interaction between CD36 and oxLDL is also known to induce the secretion of cytokines that recruit immune cell infiltrates. Accumulation of extracellular lipids droplets in the infarcted myocardium is known to occur as early as 2 hours post-acute MI (AMI) and progressively increase throughout the 48 hours following MI. Notably, between 6 and 12 hours post-acute MI, lipids are known to begin to disappear from the center of the infarct but persist at the periphery of older infarcts for at least 3 weeks. Equally important, maturing granulation tissue at the periphery of the infarct has been shown to contain moderate number of macrophages laden with lipid droplets. To date however, the link between spatial pattern of extracellular lipid accumulation in the subacute MI and adipose tissue in the old MI was unknown. It was also unclear whether these lipid-laden macrophages carry the lipid load into the chronic phase of MI. Based on the present observations and finding of others in settings outside the heart, it is indicated that siderophages progressively oxidize the lipids from the periphery of MI territory and transform into CD36+ foam cells. Since it appears that LM progressively invades the infarct core, it is likely that the lipid substrate for CD36+ foam cell formation in the core of hemorrhagic scar, which is rich in iron but has low lipid content, stems primarily from apoptotic CD36+ foam cells. The most striking evidence supporting this notion is that bigger islands of adipose tissue from 6-month old scars colocalize with iron only along the adipose tissue border pointing inwards to the core of infarct, while the central fat cells (the core of adipose tissue) is ceroid-positive but nearly iron-free. In contrast, in the 8-week old scars, individual foam cells appear to emerge from the iron-, extracellular lipid-, and ceroid-rich regions within peri-infarct and border zones of hemorrhagic MI. These findings also support that in the process of LM, iron exocytosed by foam cells as well as iron-ceroid complex from apoptotic siderophage-derived foam cells, are both recycled by newly recruited macrophage (MAC387+MΦs, where MΦs denotes unpolarized macrophages) and are progressively pushed toward the center of the myocardial scar.

Macrophage activation in response to stressors such as infection, alcohol, burn, sepsis, etc. is metabolically very expensive. Glucose is the primary fuel metabolized in proinflammatory macrophages (M1 MΦs). Hence, the proinflammatory response of M1 MΦs includes characteristic increased expression of GLUT1 and increased glucose uptake. Moreover, there is now considerable data suggesting that MΦs, which display elevated GLUT1-mediated glucose uptake and metabolism, are forced into a hyperinflammatory state with increased production of multiple inflammatory pathways and protein mediators. Notably, it has recently also been shown that glucose increases expression of the macrophage CD36 scavenger receptor. In some of the present study, we report an increased expression of GLUT1, TNF-alpha, IL-113, and CD36 markers by MΦs in hemorrhagic MI regions, particularly on larger siderophages transforming into foam cells. This indicates that the uncontrolled iron-induced M1 response/phenotype is maintained chronically by increased GLUT1-mediated glucose uptake and metabolism, which further adds to the vicious cycle of foam cell formation and fatty myocardial degeneration. Conversely, knowing that M2 MΦs are primarily dependent on β-oxidation of fatty acids for energy generation, it appears that the inability to fully switch to an M2 phenotype underlies the intracellular accumulation of lipids, as opposed to their utilization.

The role of cardiac mast cells in mediating post-infarction adverse myocardial remodeling has become of interest. Mast cells exert their physiological and pathological functions by secreting cytoplasmic granules containing a variety of mediators (proteoglycans, histamine, proteases, and proinflammatory cytokines). These biologically active mediators are released upon mast cell activation and influence the local tissue microenvironment. Activated mast cells are believed to trigger cholesterol uptake by macrophages and promote their conversion into foam cells in vitro. Furthermore, in vitro studies have shown that mast cells could prevent cholesterol efflux from foam cells. Our findings also indicate that conversion of siderophages into foam cells in hemorrhagic MIs is fine-tuned by persistently activated/degranulated mast cells, based on an understanding that iron is a potent mast cell activator.

Overall, the findings here elucidate the underpinnings of how hemorrhage within MI drives adverse remodeling, with observational studies showing that hemorrhagic MI (a) disposes the heart to infiltration of fat within the MI; ((b) hearts with fatty MI generate weaker local circumferential strain in the MI zone; and (c) increasing levels of fat within the MI overwhelms the compensatory remodeling of the heart with functional collapse that define heart failure. Our interventional studies demonstrated that an intracellular iron chelator administered up to 8 weeks post MI can (a) reduce the fat content within MI; (b) increase circumferential strain in the MI zone; and (c) drive the heart away from functional collapse, compared to control groups with the same MI size and extent of hemorrhage. These studies collectively demonstrate that the fatty remodeling of hearts following hemorrhagic MI accelerates LV remodeling in reperfused hemorrhagic MI; and that reducing the abnormal iron from hemorrhage MI zones can rescue the hearts from progressing to heart failure in the post MI period.

Methods of reducing iron deposition, proinflammatory burden within MI, and/or adverse cardiac remodeling, thereby treating the symptoms of myocardial infarction, in a subject having undergone reperfusion following myocardial infarction is provided, which include administering an effective amount of a ferric iron chelator (e.g., deferiprone, desferrioxamine, deferasirox, or a combination thereof) days after reperfusion (e.g., 3, 4, 5, 6, or 7 days after reperfusion; from 1 week to 3 months, or 4 months, 5 months, or 6 months and longer), so as to reduce iron deposition in the infarct area, reduce proinflammatory burden in the infarct area, or reduce adverse cardiac remodeling. In some aspects the methods do not include administering a ferrous iron chelator (e.g., 2,2-bipyridl or dexrazonxane) during the chronic phase period.

Other methods of reducing iron deposition, proinflammatory burden within MI, and/or adverse cardiac remodeling, thereby treating the symptoms of myocardial infarction, in a subject include administering an effective amount of a ferric iron chelator (e.g., deferiprone, desferrioxamine, deferasirox, or a combination thereof) at the chronic phase of MI, e.g., chronic phase characterized by the presence of Fe³⁺ iron crystals in macrophages in the infarction area and/or at least 7 days, and more preferably at least 2 weeks, after the onset of MI, so as to reduce iron deposition in the infarct area, reduce proinflammatory burden in the infarct area, or reduce adverse cardiac remodeling. In some aspects of the methods, while ferric iron chelator is administered, ferrous iron chelator is not administered during the chronic phase period.

Yet other methods of reducing iron deposition, proinflammatory burden within MI, and/or adverse cardiac remodeling after myocardial infarction (MI), thereby treating the symptoms of myocardial infarction, in a subject include administering an effective amount of a ferric iron chelator (e.g., deferiprone, desferrioxamine, deferasirox, or a combination thereof) at or no earlier than the sub-acute phase of MI, e.g., sub-acute phase characterized by presence of Fe³⁺ iron in the infarct area and/or about 1-2 weeks after onset of MI, so as to reduce iron deposition in the infarct area, reduce proinflammatory burden in the infarct area, or reduce adverse cardiac remodeling.

In some aspects, the subject is a subject exhibiting symptoms of or having experienced intramyocardial hemorrhage in the methods above of reducing iron deposition, proinflammatory burden within MI, and/or adverse cardiac remodeling after myocardial infarction (MI), thereby treating the symptoms of myocardial infarction. In another aspect, the subject has hemorrhagic myocardial infarction. In yet another aspect, the subject has reperfusion-induced hemorrhage with myocardial infarction.

Further aspects of these methods include selecting a subject having experienced intramyocardial hemorrhage with myocardial infarction, and administering an effective amount of a ferric iron chelator (e.g., deferiprone, desferrioxamine, deferasirox, or a combination thereof) days after reperfusion, at the chronic phase or no earlier than the sub-acute phase of MI. Other aspects include selecting a subject showing symptoms of or having been diagnosed with myocardial infarction, optionally further been treated with reperfusion, and administering an effective amount of a ferric iron chelator to the subject in need thereof, preferably at the chronic phase of MI, or no earlier than the sub-acute phase of MI.

In various aspects, the size of myocardial infarction is not increased compared to before the administration of the ferric iron chelator, in the methods above of treating or reducing the severity of hemorrhagic myocardial infarction and/or reducing the severity or likelihood of adverse cardiac remodeling. In other aspects, the size of myocardial infarction is similar to that before the administration of the ferric iron chelator, in the methods above for treating or reducing the severity of hemorrhagic myocardial infarction and/or reducing the severity or likelihood of adverse cardiac remodeling does not change. In further aspect, EDTA is not administered to the subject in the methods above. In yet some aspects, fat fraction within MI relative to the remote myocardium in a subject administered with a ferric iron chelator does not increase over time after hemorrhagic myocardial infarction (e.g., between day 3 and 8 weeks), whereas a control subject with hemorrhagic myocardial infarction not having been administered with an effective amount of a ferric iron chelator has statistically or detectably increased fat fraction within MI relative to remote myocardium. In yet some aspects, infarct-area cardiac wall thickness in a subject administered with a ferric iron chelator increases over time after hemorrhagic myocardial infarction, e.g., significantly larger by six months following ferric iron chelator therapy compared to before or at the beginning of the therapy. In further aspects, a subject administered with a ferric iron chelator has reduced LM in the MI area and improved left ventrical ejection fraction, or compared to the subject before administration of the ferric iron chelator, compared to a control subject having the symptom but not having been administered with a ferric iron chelator.

Diagnostics

One or more methods for diagnosis or determining the presence of myocardial hemorrhage in a subject are provided, including one or more medical imaging techniques, a cardiac troponin measurement, or a combination thereof, which are often performed repeatedly over several hours, days or weeks following signs or symptoms of myocardial infarction, or following a reperfusion therapy, and in various embodiments which are also performed before signs or symptoms of myocardial infarcation or before the reperfustion therapy to establish a baseline.

In some embodiments, methods for diagnosis or determining the presence of intramyocardial hemorrhage following reperfusion in a subject comprise measuring a troponin level or a cardiac troponin level of the subject for one, two or more times over a time span following the reperfusion therapy.

In some embodiments, these methods further comprise performing one or more medical imaging techniques (MRI, PET) at the heart for one, two or more times over a second time span following the reperfusion therapy, and the second can be the same or different from the time span for the measuring of the troponin (or cardiac troponin) level.

In other embodiments, these methods comprising measuring a troponin (or cardiac troponin) level also exclude (or replace) the medical imaging techniques. Therefore, in some embodiments, methods for diagnosis or determining the presence of intramyocardial hemorrhage following reperfusion in a subject consists of measuring a troponin level or a cardiac troponin level of the subject for one, two or more times over a time span following the reperfusion therapy.

In further embodiments, methods for diagnosis or determining the presence of intramyocardial hemorrhage following reperfusion in a subject comprise measuring a higher troponin level or a cardiac troponin level of the subject for one, two or more times over a time span following the reperfusion therapy, compared to a baseline level of troponin or cardiac troponin or compared to a troponin level at a reference time point. In further embodiments, methods for diagnosis or determining the presence of intramyocardial hemorrhage following reperfusion in a subject consists of measuring a higher troponin level or a cardiac troponin level of the subject for one, two or more times over a time span following the reperfusion therapy, compared to a baseline level of troponin or cardiac troponin or compared to a troponin level at a reference time point.

In various embodiments, the time span for measuring troponin level post reperfusion is 1-12 hours, 12-24 hours, 24-72 hours, or 5 days-7 days following a reperfusion therapy. In one embodiment, the methods include measuring at least once a troponin level within 12 hours following reperfusion. In another embodiment, the methods include measuring a troponin level at least once within 12 hours following reperfusion, and at least once between 12-24 hours following reperfusion. In yet another embodiment, the methods include measuring a troponin level at least once within 12 hours following reperfusion, at least another once between 12 and 24 hours following reperfusion, and yet at least another once between 24 and 72 hours following reperfusion. In yet another embodiment, the methods include measuring a troponin level at least once within 24 hours following reperfusion and at least another once between 24 and 72 hours following reperfusion. In yet another embodiment, the methods include measuring a troponin level (1) at least once within 12 hours following reperfusion and at least once between 12 and 24 hours following reperfusion; or at least once within 24 hours following reperfusion; and one or more of: (2) at least once between 24 and 72 hours following reperfusion, (3) at least once between 3 days and 5 days following reperfusion, (4) at least once between 5 days and 7 days following reperfusion. In a further embodiment, the methods include measuring a troponin level (1) at least once within 12 hours following reperfusion and at least once between 12 and 24 hours following reperfusion; or at least once within 24 hours following reperfusion; and two or all three of: (2) at least once between 24 and 72 hours following reperfusion, (3) at least once between 3 days and 5 days following reperfusion, (4) at least once between 5 days and 7 days following reperfusion.

In various embodiments, a higher than baseline level of cardiac troponin level measured within 12 hours following reperfusion is indicative of the presence of an intramyocardial hemorrhage. In various embodiments, a higher than baseline level of cardiac troponin level measured within 24 hours following reperfusion is indicative of an intramyocardial hemorrhage. In further embodiments, the higher than baseline level is at least 7 times, 6.5 times, 6 times, 5.5 times, 5 times, 4.5 times, 4 times, or 3 times higher at a time frame within 12 hours following reperfusion therapy, for a human subject, to indicate the presence of IMH; whereas a human subject with no IMH following a reperfusion therapy would at most have about 2 times higher level of cardiac troponin at a time frame within 12 hours following reperfusion therapy, compared to his/her own baseline level. Generally, a baseline level is one obtained prior to reperfusion of the same subject; or obtained within 24 hours prior to reperfusion; or one obtained following onset of myocardial infarction signs or symptoms but before reperfusion therapy. In some embodiments, the methods include measuring a blood level of troponin or cardiac troponin of the subject at two different time points after the coronary re-vasucularization or the reperfusion therapy. In some embodiments, the methods include measuring a blood level of troponin or cardiac troponin of the subject at three different time points after the coronary re-vasucularization or the reperfusion therapy. In some embodiments, the methods include measuring a blood level of troponin or cardiac troponin of the subject at four different time points after the coronary re-vasucularization or the reperfusion therapy. In some embodiments, the methods include measuring a blood level of troponin or cardiac troponin of the subject at five different time points after the coronary re-vasucularization or the reperfusion therapy. In some embodiments, the methods include measuring a blood level of troponin or cardiac troponin of the subject at six different time points after the coronary re-vasucularization or the reperfusion therapy. In some embodiments, the methods include measuring a blood level of troponin or cardiac troponin of the subject at seven different time points after the coronary re-vasucularization or the reperfusion therapy. In further embodiments, the methods include administering a treatment to the subject to control hemorrhage from the cardiac chamber of the subject, when the blood level of troponin or cardiac troponin rises to a level that is between 3 times and 5 times higher than that before the coronary re-vascularization or the reperfusion therapy in the subject. In other embodiments, the methods include administering a treatment to the subject to control hemorrhage from the cardiac chamber of the subject, when the blood level of troponin or cardiac troponin rises to a level that is between 4 times and 6 times higher than that before the coronary re-vascularization or the reperfusion therapy in the subject. In other embodiments, the methods include administering a treatment to the subject to control hemorrhage from the cardiac chamber of the subject, when the blood level of troponin or cardiac troponin rises to a level that is between 5 times and 7 times higher than that before the coronary re-vascularization or the reperfusion therapy in the subject. In other embodiments, the methods include administering a treatment to the subject to control hemorrhage from the cardiac chamber of the subject, when the blood level of troponin or cardiac troponin rises to a level that is between 3 times and 6 times higher than that before the coronary re-vascularization or the reperfusion therapy in the subject. In further embodiments, the methods include administering a treatment to the subject to control hemorrhage from the cardiac chamber of the subject, when the blood level of troponin or cardiac troponin rises to a level that is between 3 times and 7 times, between 3 times and 8 times, between 3 times and 9 times, between 3 times and 10 times, between 4 times and 7 times, between 4 times and 8 times, between 4 times and 9 times, between 4 times and 10 times, between 5 times and 8 times, between 5 times and 9 times, between 5 times and 10 times, between 6 times and 8 times, between 6 times and 9 times, between 6 times and 10 times, between 7 times and 9 times, between 7 times and 10 times, between 8 times and 10 times, between 9 times and 10 times, or at least 10 times, higher than that before the coronary re-vascularization or the reperfusion therapy in the subject.

In some embodiments, a higher cardiac troponin level measured within 12 hours following reperfusion, compared to the level measured between 12 and 24 hours following reperfusion, in a human subject with myocardiac infarction and having undergone a referpusion therapy (e.g., percutaneous coronary intervention), is indicative of the presence of an intramyocardial hemorrhage. In further embodiments, a higher cardiac troponin level measured between 12 and 24 hours following reperfusion, compared to the level measured between 24 and 72 hours following reperfusion, in the human subject is indicative of the presence of an intramyocardial hemorrhage. In contrast, various embodiments provide that a lower cardiac troponin level measured within 12 hours following reperfusion, compared to the level measured between 12 and 24 hours following reperfusion, in a human subject with myocardiac infarction and having undergone a referpusion therapy is indicative of an absence of intramyoardial hemorrhage in the subject. Accordingly, in some embodiments, a reference time point of troponin level can be an immediate previous measurement time point; or in other embodiments, a reference time point can be an immediate next measurement time point.

In further embodiments, a subject determined to have intramyocardiac hemorrhage is subjected to a treatment to control hemorrhage from a cardiac chamber, such as an iron chelators, heme blockers and inactivators, a hemostatic agent, using carbon monoxide-releasing molecules (CORMs, such as metal carbonyl complexes, Ru(glycinate)Cl(CO)₃) to prevent heme from breaking up, or a combination thereof.

Treatment & Monitoring Following Diagnostics

One or more methods are also provided for treating hemorrhagic myocardial infarction in a subject, and/or mitigating infarct expansion in a subject with hemorrhagic myocardial infarction. While some current standard practices provide anti-platelet therapies before PCI and/or on the PCI table (during or immediately before and following PCI procedure), the disclosed methods identifying hemorrhagic infarction based on measurements of troponin level over time (sampling) can introduce the iron chelator therapies, or in combination with one or more anti-inflammatory agents, in subsequent treatments of the subject, so as to mitigate infarct size expansion and ultimately improves myocardial remodeling.

In some embodiments, a method for treating hemorrhagic myocardial infarction in a subject, and/or mitigating infarct expansion in a subject with hemorrhagic myocardial infarction comprises: diagnosing the subject as having had a hemorrhagic myocardial infarction by detecting from blood samples of the subject a peak level of troponin within 18 hours following a reperfusion therapy and/or an increase in the level of troponin by at least 1.5 ng/mL or greater within 18 hours following the reperfusion therapy compared to a level before the reperfusion therapy, and administering an effective amount of one or more iron chelators, an anti-inflammatory agent, or a combination thereof, to the subject as described above.

In some embodiments, a method for treating hemorrhagic myocardial infarction in a subject, and/or mitigating infarct expansion in a subject with hemorrhagic myocardial infarction comprises: diagnosing the subject as having an on-going hemorrhagic myocardial infarction by detecting from blood samples of the subject an increase in troponin level at 0.4 ng/mL/hr or greater within 12 hours following a reperfusion therapy, and administering an effective amount of one or more iron chelators, an anti-inflammatory agent, or a combination thereof, to the subject as described above.

In some embodiments, a method for treating hemorrhagic myocardial infarction in a subject, and/or mitigating infarct expansion in a subject with hemorrhagic myocardial infarction comprises: obtaining the results of an analysis of blood levels of troponin over time following a reperfusion therapy, and administering an effective amount of one or more iron chelators, an anti-inflammatory agent, or a combination thereof, to the subject as described above when the blood levels of troponin peak within 18 hours following the reperfusion therapy and/or the blood levels of troponin increase by at least 1.5 ng/mL greater within 18 hours following the reperfusion therapy compared to a level before the reperfusion therapy.

In some embodiments, a method for treating hemorrhagic myocardial infarction in a subject, and/or mitigating infarct expansion in a subject with hemorrhagic myocardial infarction comprises: obtaining the results of an analysis of blood levels of troponin over time following a reperfusion therapy, and administering an effective amount of one or more iron chelators, an anti-inflammatory agent, or a combination thereof, to the subject as described above when the blood levels of troponin increase at 0.4 ng/mL/hr or greater within 12 hours following a reperfusion therapy.

In some embodiments, a method for treating hemorrhagic myocardial infarction in a subject, and/or mitigating infarct expansion in a subject with hemorrhagic myocardial infarction comprises: requesting the results of an analysis of blood levels of troponin over time following a reperfusion therapy, and administering an effective amount of one or more iron chelators, an anti-inflammatory agent, or a combination thereof, to the subject as described above when the blood levels of troponin peak within 18 hours following the reperfusion therapy and/or the blood levels of troponin increase by at least 1.5 ng/mL or greater within 18 hours following the reperfusion therapy compared to that before the reperfusion therapy.

In some embodiments, a method for treating hemorrhagic myocardial infarction in a subject, and/or mitigating infarct expansion in a subject with hemorrhagic myocardial infarction comprises: requesting the results of an analysis of blood levels of troponin over time following a reperfusion therapy, and administering an effective amount of one or more iron chelators, an anti-inflammatory agent, or a combination thereof, to the subject as described above when the blood levels of troponin increase at 0.4 ng/mL/hr or greater within 12 hours following a reperfusion therapy.

In some embodiments, a method for treating hemorrhagic myocardial infarction in a subject, and/or mitigating infarct expansion in a subject with hemorrhagic myocardial infarction comprises: administering an effective amount of one or more iron chelators, an anti-inflammatory agent, or a combination thereof, as described above, to the subject who has been determined to have a blood level of troponin that peaks within 18 hours after the subject receives a reperfusion therapy, that increases by at least 1.5 ng/mL or greater within 18 hours following the reperfusion therapy compared to a level before the reperfusion therapy.

In some embodiments, a method for treating hemorrhagic myocardial infarction in a subject, and/or mitigating infarct expansion in a subject with hemorrhagic myocardial infarction comprises: administering an effective amount of one or more iron chelators, an anti-inflammatory agent, or a combination thereof, as described above, to the subject who has been determined to have a blood level of troponin that increases at a rate of 0.4 ng/mL/hr or greater within 12 hours following a reperfusion therapy.

A “peak” of the troponin level refers to an increase followed by a decrease, and in some embodiments refers to that the level rises to a highest point followed by decrease within a specific time frame.

Various embodiments provide for methods for inhibiting the extent, or reducing the likelihood, of myocardial infarct expansion in a subject diagnosed with, suffering, or having had myocardial infarction, which include (A) administering a composition comprising a ferrous iron chelator, or a combination of a ferrous iron chelator and a ferric iron chelator, or an iron chelator, at an appropriate time to the subject; and (B) measuring a troponin level (or cardiac troponin level) over a time span, and/or performing a medical cardiac imaging, to determine the presence or absence of intramyocardial hemorrhage in the subject, especially following a reperfusion therapy. Further embodiments provide the methods include (C) administering a composition comprising an iron chelator and/or a hemostatic agent to the subject when the subject is determined to have intramyocardial hemorrhage.

Further embodiments provide for methods for inhibiting the extent, or reducing the likelihood, of myocardial infarct expansion in a subject in need of or having had a percutaneous coronary intervention for a heart condition such as coronary artery disease, which include (A) administering a composition comprising a ferrous iron chelator, or a combination of a ferrous iron chelator and a ferric iron chelator, or an iron chelator, at an appropriate time to the subject; and (B) measuring a troponin level (or cardiac troponin level) over a time span, and/or performing a medical cardiac imaging, to determine the presence or absence of intramyocardial hemorrhage in the subject. In additional embodiments, the methods include (C) administering a composition comprising an iron chelator and/or a hemostatic agent to the subject when the subject is determined to have intramyocardial hemorrhage.

In some embodiments, methods for reducing myocardial infarct size, and/or inhibiting expansion of the myocardial infarct size, in a subject with hemorrhagic myocardial infarction, or a subject having had a reperfusion therapy, or a subject at risk of developing intramyocardial hemorrhage, comprise (A) administering a composition comprising an effective amount of a ferrous iron chelator, an agent that binds heme, an agent that regulates heme, or a combination thereof, during the acute phase or within 3 days of the onset of myocardial infarction; (B) measuring a blood level of troponin or cardiac troponin of the subject before and after coronary re-vascularization or the reperfusion therapy, or at two or more time frames after the coronary re-vasucularization or the reperfusion therapy; and (C) administering a treatment to the subject to control hemorrhage from the cardiac chamber of the subject, when the blood level of troponin or cardiac troponin rises within 30 minutes to 12 hours following the coronary re-vascularization or the reperfusion therapy, which is at least 7 times, 6.5 times, 6 times, 5 times, 4 times, or 3 times higher compared to the level in the subject before the coronary re-vascularization or the reperfusion therapy; or in (C) no treatment to control hemorrhage from the cardiac chamber is administered to the subject, when the blood level of troponin or cardiac troponin within 30 minutes to 12 hours following the coronary re-vascularization or the reperfusion therapy is not higher, or less than 3 times higher, compared to the level in the subject before the coronary re-vascularization or the reperfusion therapy.

In some embodiments, the composition comprising a ferrous iron chelator, an agent that binds heme, an agent that regulates heme, or a combination thereof, is administered to the myocardium of the subject, or to the heart of the subject, or to the circulation system to reach the heart of the subject.

Detections

One or more methods of detecting a level of troponin in a subject are also provided.

In some embodiments, a method of detecting a level of troponin in a subject with myocardial infarction and undergoing or having undergone a reperfusion therapy, comprises assaying a biological sample (e.g., blood) obtained from the subject, wherein the subject desires a determination regarding hemorrhagic myocardial infarction; and detecting the level of troponin over time within 18 hours following a reperfusion therapy.

In some embodiments, a method of detecting a level of troponin in a subject with myocardial infarction and undergoing or having undergone a reperfusion therapy, comprises assaying a biological sample obtained from the subject, wherein the subject desires a determination regarding hemorrhagic myocardial infarction; and detecting an increased level of troponin by 1.5 ng/mL or greater within 18 hours following a reperfusion therapy compared to a level before the reperfusion therapy, or detecting an increase in the level of troponin by a rate of 0.4 ng/mL/hr or greater within 12 hours following the reperfusion therapy.

In some embodiments, a method of detecting a level of troponin in a subject with myocardial infarction and undergoing or having undergone a reperfusion therapy, comprises assaying a biological sample (e.g., blood) obtained from the subject, wherein the subject exhibits a symptom of hemorrhagic myocardial infarction; and detecting the level of troponin over time within 18 hours following a reperfusion therapy.

In some embodiments, a method of detecting a level of troponin in a subject with myocardial infarction and undergoing or having undergone a reperfusion therapy, comprises assaying a biological sample (e.g., blood) obtained from the subject, wherein the subject exhibits a symptom of hemorrhagic myocardial infarction; and detecting an increased level of troponin by 1.5 ng/mL or greater within 18 hours following a reperfusion therapy compared to a level before the reperfusion therapy, or detecting an increase in the level of troponin by a rate of 0.4 ng/mL/hr or greater within 12 hours following the reperfusion therapy.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1. Hemorrhage Drives a Chain of Time-Sensitive Events which Continually Damage the Heart Via Infarct Expansion in Acute Phase and Persistent Proinflammatory Burden in Chronic Phase of MI

Design and Rationale: Patients with hMI are at the greatest risk for MACE. Large reperfused acute MIs are often accompanied by hemorrhage. In the chronic phase, MIs with reperfusion hemorrhage are liked to prolonged proinflammatory burden. Both MI size and prolonged inflammatory burden are key predictors adverse LV remodeling, which culminates in CHF. Yet, the core biology contributing to these observations are not known.

Preliminary Data:

(a) Reperfused MIs with hemorrhage are larger than non-reperfused MIs.

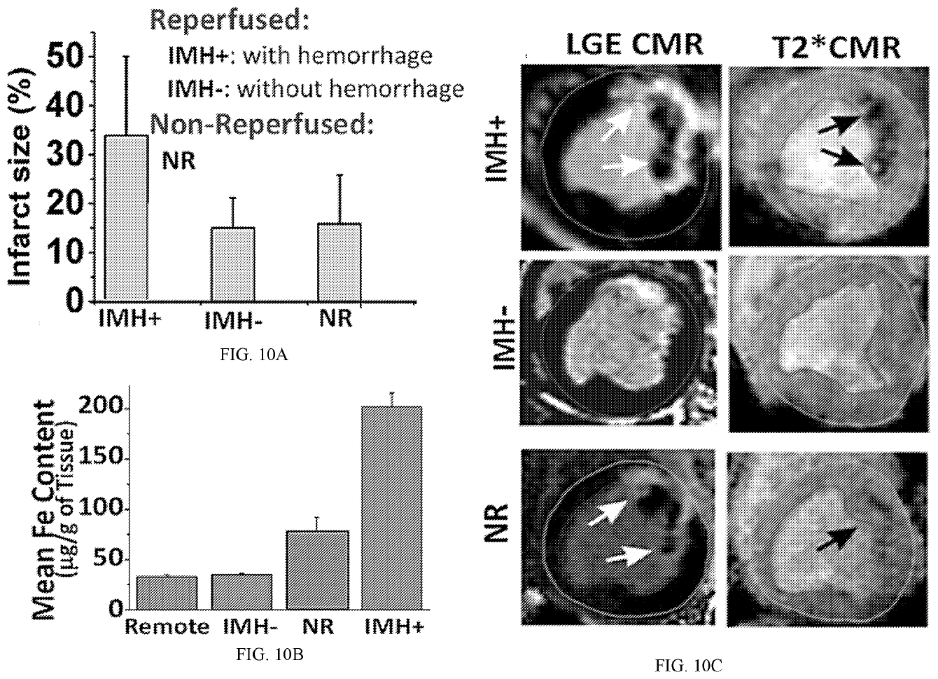

A canonical notion in cardiac pathophysiology is that acute MI size is limited to initial zone of ischemia (area-at-risk, AAR). Given that hMIs are often large, Applicant hypothesized that hemorrhage can drive MI size beyond the AAR due to the exposure of cardiomyocytes to large levels of toxic heme-iron. Dogs (n=40) underwent no-flow ischemia of LAD (below the first diagonal) for 3 hrs; 20 animals were then reperfused and others were not. CMR was performed on weeks 1 and 8 post MI to assess MI size (LGE) and hemorrhage/iron (T2*). MI size was compared between reperfused animals with (MI+) and without hemorrhage (IMH−), and non-reperfused (NR) groups. NR groups served as the control for AAR unaltered by reperfusion injury. Mass spectrometry was used to quantify the amount of iron within MI and remote territories. All dogs developed MIs but the size of hMIs was >2-fold greater than non-hMI or NR animals (p<0.01). Size of hemorrhage and MI were highly correlated (R=0.8, p<0.01) (see FIGS. 10A-10C). Non-hMIs were smaller than NR MIs. hMIs had larger than 10-fold greater iron content than non-hMI and 4-fold greater iron than NR. Summary: Reperfusion may do more harm than good, if it leads to hemorrhage. Notably, the size of MIs with hemorrhage are larger than the associated AAR; and the iron content of hMI is significantly larger than all other MI types. Applicant tests whether heme-iron drives infarct expansion in hMI.

(b) Validation and reproducibility of clinically relevant rat model of hMI.

Occurrence of hemorrhage within reperfused MIs in large animals and patients is known; but, this has not been shown in rats. Methods: 60 animals underwent either a 90-min ischemia and then reperfusion (I/R) (n=22), a 60-min I/R (n=20) or 30-min I/R (n=18) of the LAD. About 80% of 90-min I/R and all rats from other groups survived. 24-hrs after reperfusion rats were injected with Gd and sacrificed. LGE and T2* CMR were performed on excised hearts in a 9.4T scanner. H&E verified tissue damage detected on LGE; and Prussian Blue (PB) stains verified presence of iron on T2*-CMR. Results are shown in FIG. 10D. All animals with 90-min I/R were positive for hMI and all with 30-min I/R were not hemorrhagic (non-hMI). In 60-min I/R, 12 animals developed hMI but 8 animals were non-hMIs. Summary: 90-min I/R always yields hMI, while 30-min I/R consistently yields non-hMI without no reflow. But, 60-min I/R results in both hMIs (60%) and non-hMI (40%), with both groups having no reflow. Applicant uses these models to evaluate the differential effects of hemorrhage from non-hMI with and without no-reflow.

(c) Rats exposed to 90-min I/R demonstrate marked oxidative stress, upregulation of ferroptosis markers in the peri-infarct zone in the acute phase and persistent upregulation of autophagy throughout post MI.

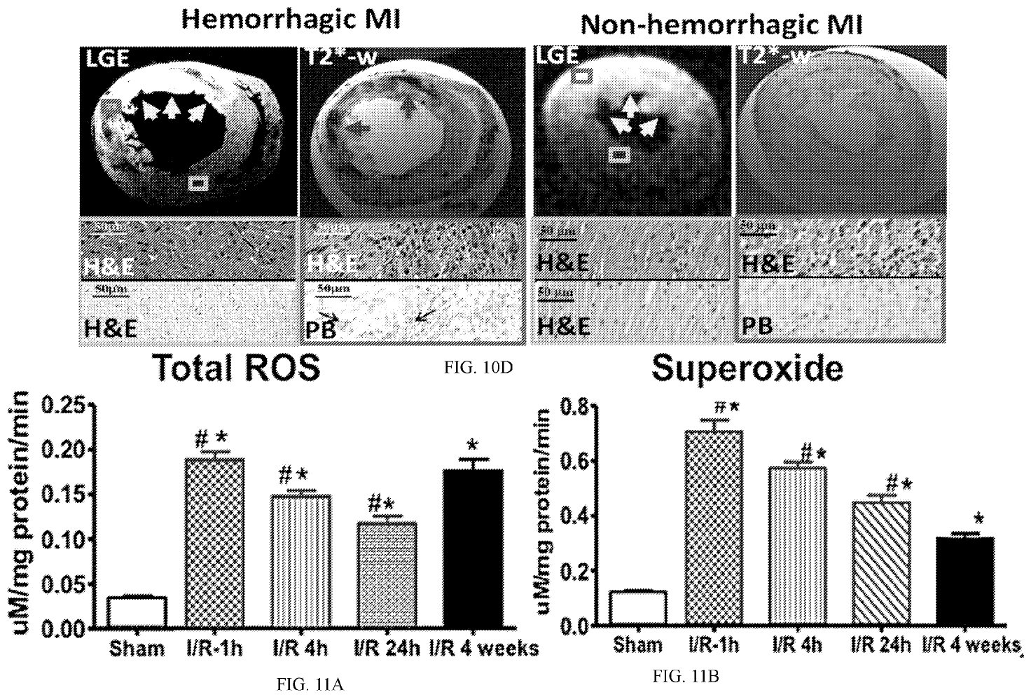

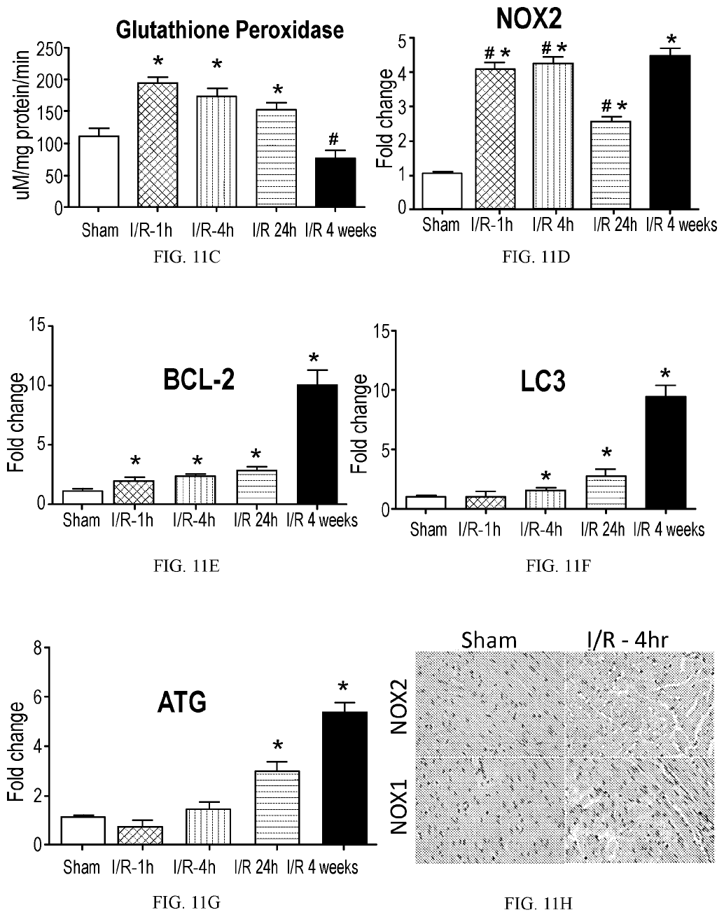

Applicant investigated oxidative stress, ferroptosis and autophagy in the pen-infarct zone in rats undergoing 90-min I/R. Methods: Rats (n=15) were subjected to 90-min I/R (3 animals died) or sham operation (n=12). Animals (3 per group) were sacrificed at 1, 4, 24 hrs and 4 wks post reperfusion or sham operation. Oxidative stress and ferroptosis markers (malonaldehyde antioxidant enzyme glutathione peroxidase and NOX), and autophagic genes were measured in the peri-infarct regions or sham hearts. Results: Preferentially elevated oxidative stress, ferroptosis markers and autophagic genes and decreasing antioxidant levels were observed in the pen-infarct zones (FIGS. 11A-11H). Conclusion: 90-min I/R results in marked oxidative stress and upregulation of ferroptosis in the super acute phase and persistent upregulation of autophagy in acute and chronic phases of MI. Since 90-min I/R results in hMI, this indicates that hMIs are exposed to extensive tissue damage throughout post MI period. Applicant establishes and extends these findings to show that hemorrhage promotes extensive ferroptosis of cardiomyocytes in the peri-infarct zone; and prolonged upregulation of autophagy. (d) Hemopexin reduces oxidative stress and improves LV remodeling in rats with 90-min I/R.

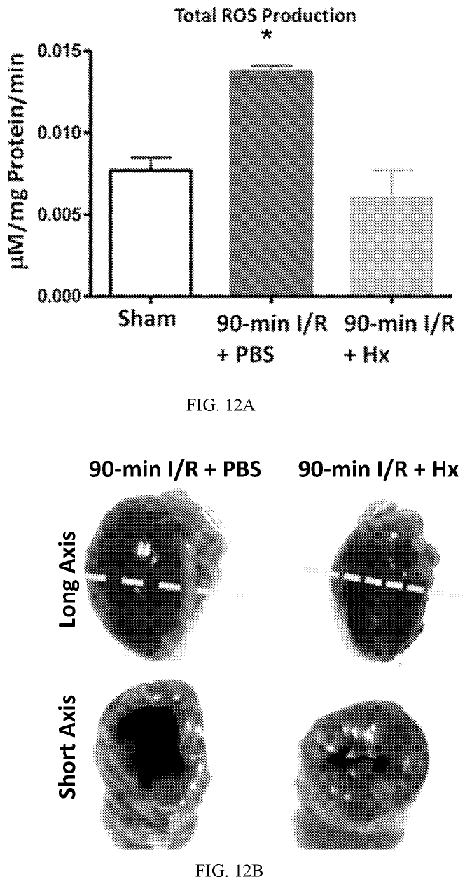

Background: Hemopexin (Hx), a known scavenger of heme, and a key byproduct of hemorrhage. Applicant tested whether treating rats with 90-min I/R (known to yield hMI) with Hx could reduce oxidative stress in the acute phase and improve LV remodeling in the chronic phase. Methods: Rats (n=12) underwent 90-min I/R: 50% of the animals were treated with Hx (700 μg, i.p. immediately and 2 days post reperfusion) and the rest were treated with PBS. LV function was determined with echo. Half of each group was sacrificed at 24-hrs and the rest were sacrificed at 1-month post MI. Results: Total ROS was significantly lower in Hx vs. control group at 24-hrs (see FIGS. 12A and 12B). LVEF were: 51±4% (Hx+) vs 32±5% (control), p<0.05. Summary: Heme-specific scavenger, Hx, can attenuate the adverse effects of hMI.

Research Methods: Applicant performs two tasks: Task 1 will test whether hemorrhage promotes infarct expansion in the acute phase through ferroptosis of cardiomyocytes in the infarct periphery; and Task 2 will test whether the iron, a byproduct of hemorrhage, in the chronic phase polarizes the macrophages, which attempt to clear them, to produce inflammatory cytokines that impair scar formation and cause adverse remodeling. The endpoints of this study are to show that hemorrhage within MI imposes the largest reperfusion injury in the acute phase (<3 days post reperfusion) compared to non-hMIs; and that hemorrhage within MI mediates a persistent proinflammatory phenotype in the chronic phase which support adverse LV remodeling. Animal Groups: Reperfused MIs with and without hemorrhage will be created using a 60-min I/R protocol; expected yields 60% with and 40% without hemorrhage. This allows us to study the effects of hemorrhage without being confounded by ischemia duration preceding reperfusion.

Task 1: Ferroptosis as a mechanism for infarct expansion in the acute phase of hMI.

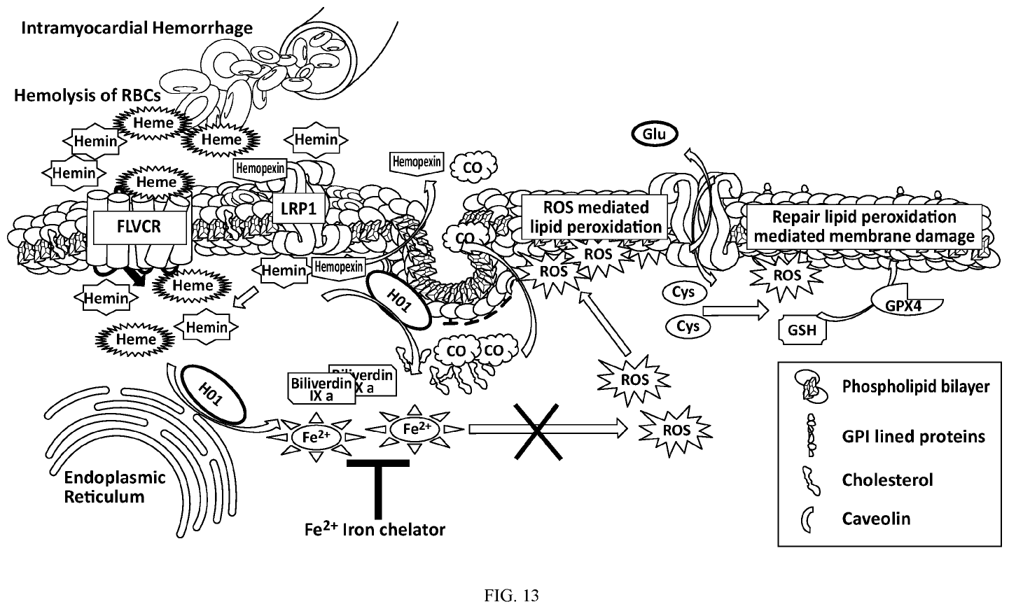

Applicant tests whether ferroptosis drives infarct expansion in hMI. Ferroptosis is caused by loss of activity of the key enzyme that is tasked with repairing oxidative damage to cell membranes—glutathione peroxidase 4 (GPX4); See FIG. 13. Normally, cysteine (substrate for glutathione (GSH) synthesis) is imported into the cell in exchange for glutamate. When this step is inhibited, GSH is depleted and phospholipid peroxidase, GPX4 is inactivated. GPX4 converts potentially toxic lipid hydroperoxides (L-OOH) to non-toxic lipid alcohols (L-OH). Inactivation of GPX4 through depletion of GSH with erastin, or with the direct GPX4 inhibitor (1S,3R)-RSL3 (also known as RSL3), ultimately results in overwhelming lipid peroxidation that causes cell death. GPX4 normally removes the dangerous products of iron-dependent lipid peroxidation, protecting cell membranes from this damage; when GPX4 fails, ferroptosis ensues. To confirm that infarct expansion is mediated by heme via ferroptosis, hemopexin (Hx) will be used to block the effects of heme and demonstrate that GSH depletion, as well as increased malonaldehyde and protein carbonyls levels (key markers of ferroptosis), are absent in rats with hMI treated with Hx vs. PBS. Data Collection: We will use 300 animals (including 20% attrition from un even induction of hMI): 120 hMI (60 with hemopexin (Hx+); 60 without (Hx−)); 120 non-hMIs (60 Hx+; 60 Hx−); 10 rats per time (6 time points, details below). Animals treated with Hx will be receive 700 μg of the drug, i.p. 2 times/week for 1 month.

Rats will be sacrificed at the following time points: immediately after reperfusion or thoracotomy (sham), 4 hrs, day 1, day 2, day 3, and 1-week post MI or thoracotomy. Area-at-Risk (AAR) will be determined with Evans Blue dye. Hearts will be cut into two halves (one half will be used for determination of AAR and TTC-based infarct size (IS) based on planimetry; other half will be sectioned for samples from regions of infarct periphery (BZ), infarct core (IZ) and remote (Rm)). In BZ, IZ and Rm sections ferroptosis will be assessed on the basis of GSH depletion and lipid peroxidation (protein carbonyls and malonaldehyde); and autophagy/mitophagy markers will be measured in the as described previously. Iron content in BZ, IZ and Rm will be determined using mass spectrometry as we previously described. Data AnalvsiF ROS markers, GSH levels and other specific ferroptosis markers (malonaldehyde and protein carbonyls) from BZ, IZ and Rm sections will be compared at each time point using repeated-measure ANOVA. Effectiveness of Hx in decreasing ferroptosis will be assessed based on ROS markers, GSH, protein carbonyl and malonaldehyde levels in the BZ of hMI rats (Hx+vs. Hx−). To determine whether hMI promotes infarct expansion, relative change in IS/AAR between immediately after reperfusion and week 1 post MI will be studied in hMI and non-hMI groups. To determine whether heme promotes infarct expansion, we will test whether: (i) IS/AAR of hMI/Hx->IS/AAR of hMI/Hx+; and (ii) the change in IS/AAR in hMI/Hx+ group is not different from non-hMI group. Differences in iron content in BZ between the hMI/Hx- and hMI/Hx+ will be compared at each time points via paired t-tests.

Data Interpretation: From preliminary data showing that hMIs are larger than non-reperfused infarcts, it is conceived that IS/AAR of hMI/Hx->hMI/Hx+; and that the relative change in IS/AAR between right after reperfusion and week 1 in hMI/Hx->hMI/Hx+ groups. Since heme is known to promote oxidative stress and upregulate ferroptosis genes, it is conceived NOX in BZ of hMI>NOX in BZ of non-hMI, but not different in IZ or Rm. If heme causes ferroptosis of myocytes in the BZ, iron concentration in BZ in hMI>non-hMI at week 1. If infarct expansion proceeds via ferroptosis, BZ will exhibit elevated levels of ferroptosis markers (malonaldehyde, protein carbonyl and GPX4) in hMI compared to non-hMI around day 3, when infarct size is expected to stabilize. If heme drives ferroptosis and infarct expansion, Hx+ rats will have reduced IS/AAR and GSH transcripts compared Hx− post reperfusion. Based on preliminary data (b), all animals subjected to 60-min I/R are expected to develop no-reflow (60% with and 40% without hemorrhage). Thus comparing hMI and non-hMI animals with 60-min I/R provides an opportunity to assess the differential effects of no-reflow and hemorrhage on tissue injury. These studies can be the first to show that heme from hMI causes infarct expansion beyond the AAR and that reperfusion injury in hMI are greater than in non-hMIs with no reflow.

Hx treatment is believed to lead to high levels of Hx in extracellular space of hMI, facilitating efficient scavenging of heme in the acute phase of hMI when heme is externalized from dying red blood cells. This may not fully eliminate heme-mediated reperfusion injury as Hx dose may be suboptimal. Alternatively, if ferroptosis is evident, Applicant would supplement Hx with heme oxygenase (HO) to enable natural detoxification of heme. Although this mechanism normally functions to detoxify heme in vivo, hemorrhage likely overwhelms endogenous capacity.

Task 2: Chronic iron deposition in hMIs leads to proinflammatory cellular cascade in macrophages.

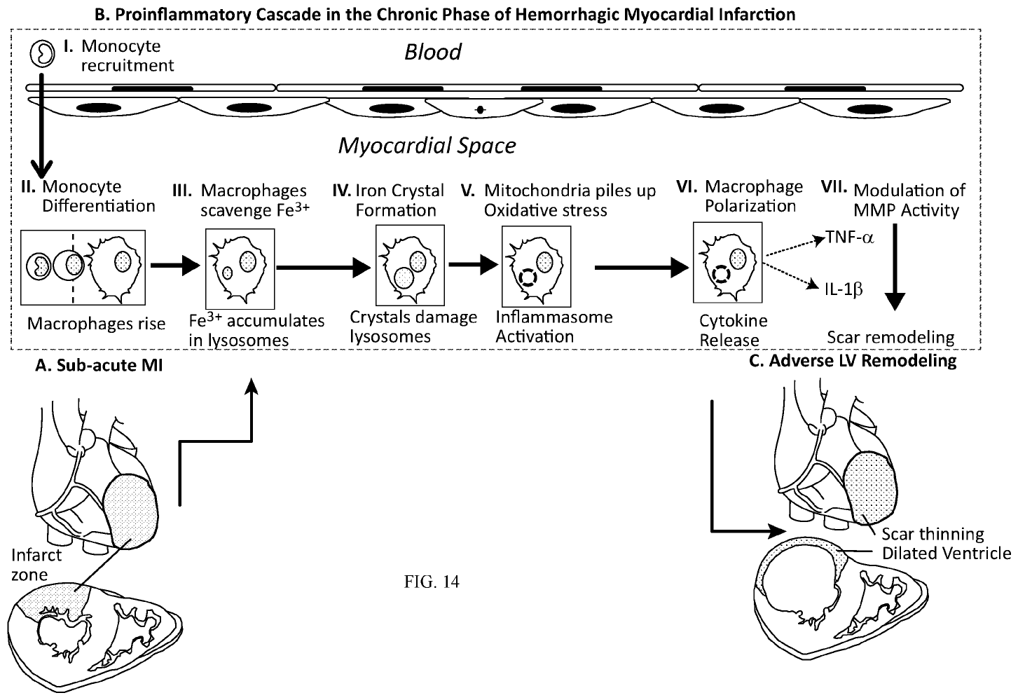

Here applicant studies whether chronic iron deposits drive a proinflammatory cascade in hMI but not in non-hMI. We will test the proposed mechanism that accumulation of iron within lysosomes of macrophages drive autophagy, oxidative stress, and mitochondria! damage which culminate in inflammasome activation and macrophage polarization toward proinflammatory (M1) than anti-inflammatory (M2) phenotype (FIG. 14). The endpoint of this Task 2 is to demonstrate that M1:M2 of hMI>MI:M2 of non-hMI at weeks 2 to 8 post MI.

Data Collection & Analysis. 100 rats will be studied (20% attrition due to uneven hMI and non-hMI yields): 40 hMI and 40 non-hMI; 10 rats per time point (see below). Rats will be euthanized at 1, 2, 3, 4 and 8 wks post MI. Autophagy: RT-PCR and Western blotting will be used to evaluate the up regulation of autophagy genes (LC3, ATG5, 12, 16, BCL2 and BECLIN). SQSTMI/p62 will be used to infer autophagic flux. Punctate LC3 will be assessed by immunostaining to quantify autophagosomes. Lysosomal leakage will be determined based on the uptake of acridine orange or other acidotropic fluorochromes and by redistribution of lysosomal cathepsins to cytosol. Oxidative stress in the mitochondria and cytosol will be measured in tissues as we previously described. Mitochondrial damage will be assessed using TEM and by cytochrome c release. Mitochondrial structure and functional changes will be measured in the heart tissue and in isolated mitochondria. Functional studies will be performed by isolating mitochondria and performing a swelling assay. Mitochondrial permeability transition will be monitored as the changes at 540 nm, in the swelling buffer in the absence or presence of Ca′. Mitochondrial respiratory function will be performed using oxygen electrode. Mitochondrial gene expression for oxidative phosphorylation (OXPHOS) will be measured using RT-PCR and protein levels will be measured using western blotting as we have done in the past. Inflammasome activation will be measured on the basis of proteolytic activation of caspase 1 by western blotting and activity assay with fluorogenic substrate. Macrophage Polarization: Since NOS2 and ARG1 are well established markers of M1 and M2 macrophages, respectively, the ratio of NOS2 to ARG1 protein expression will be determined in hMI and non-hMI territories to assess the polarization of macrophage towards proinflammatory or anti-inflammatory phenotype. Histology, immunohistochemistry, proinflammatory gene and protein expression and mass spectrometry: Histology (Prussian blue, PB) and immunohistochemistry (CD68, CD163, IL-1β TNF-α, MMPs, IL-10 and TGF-β) will be performed and quantified as described previously.

Areas positive for iron and immune markers will be regressed. qRT-PCR and Western-blot analysis will be used to study the level of differential gene and protein expression of IL-16, TNF-a, MMPs, TIMPs, IL-1I3 and TGF-a will be correlated against iron content measured from mass spectrometry as described previously. Data Interpretation: it is conceived that oxidative stress (mitochondrial and cytosolic) and autophagic genes will be elevated during acute and chronic phase of reperfusion injury. Specifically, it is conceived that in the acute phase of MI ischemia will cause hypoxia in the coronary vasculature and reperfusion will result in increased oxidative stress resulting in ferroptosis, which in turn will upregulate autophagy. In the chronic phase, the conversion of Fe′ (from heme) to Fe³⁺ will accentuate oxidative stress leading to greater activation of autophagic genes, formation of autophagosomes, impairment of lysosomal function and accumulation of damaged mitochondria in macrophages. Western blots and gene expression can provide evidence on the hypothesized relationships among iron with IL-β, TNF-α, and MMPs. Since IL-β and TNF-α are known to upregulate MMPs based on preliminary data, both cytokine and MMP levels are believed to be directly correlated with iron. These studies have the potential to provide direct mechanistic evidence implicating iron accumulation increases proinflammatory cytokines and MMPs, both which are important contributors to adverse remodeling.

Using 60-min I/R to generate hMI and non-hMI rats probably yields more hMI than non-hMI. An alternate method for creating hMIs and non-hM Is without being confounded by ischemia time is to expose rats to 30-min I/R, which would otherwise yield non-hMIs, to agents that increase capillary permeability (such as collagenase, VEGF etc.) prior to reperfusion. During the chronic phase of MI, as the heart undergoes adverse remodeling, oxidative stress, autophagy and inflammation may overlap, potentially complicating the separation of cause and effect. If macrophage death is also enhanced at the site of acute hMI, this would provide the first evidence that macrophage infiltration into the infarct core is not simply due to impenetrability of no-reflow zone but due to heme-mediated toxicity.

### Example 2. Time-Dependent Changes in the Spatial, Temporal and Biochemical Features of Iron within hMI which Facilitate Continued Myocardial Damage in the Acute and Chronic Phases of Infarction

Design and Rationale: the preliminary data supports the notion that iron from hemorrhage within MI likely mediates continuous damage to the heart in the post infarction period. Applicant hypothesizes that removing iron from the infarct zone can marginalize this damage. However, iron which starts off bound to the porphyrin ring (heme iron) undergoes multiple time-dependent changes in location (extra- vs. intra-cellular), oxidation state (ferrous vs ferric), and physical state (free, crystalline vs non-crystalline) once the red blood cells are extravasated. While iron chelators can be effective in removing the iron from tissue, not all iron chelators are the same as their chelation efficacy is tied to physicochemical features of iron. Hence, an optimal iron chelation strategy for hMI, requires the knowledge of the evolving features of iron in the post MI setting. The endpoint of this study is to determine the time-dependent features of iron within hMI (specifically, which iron chelators to use and when) so that optimal treatment strategy is possible.

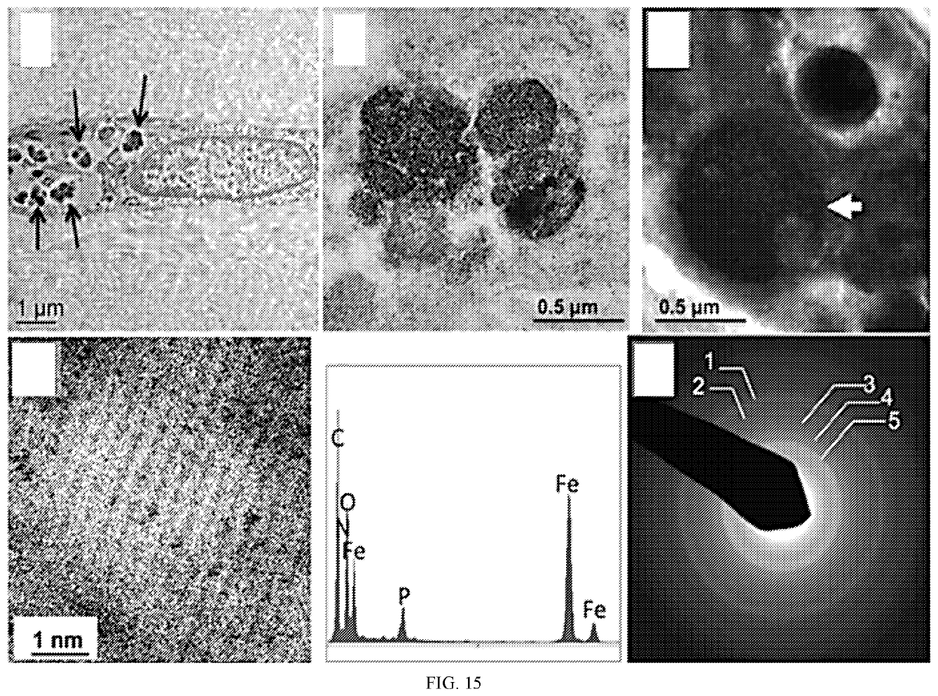

Preliminary data: Iron localization, crystallinity and oxidation state at 2-months post MI. Applicant has shown that iron deposits within chronic hMIs; but its exact location within MI and its physicochemical features are unknown. Applicant studied this in 8-wk old MI territories. Methods: High-resolution electron microscopy (TEM), atomic resolution imaging and energy dispersive spectroscopy (EDS) were performed on ex-vivo sections positive for iron. Results: TEM revealed electron-dense material within macrophages. Atomic imaging showed that this material is organized into nanocrystals (FIG. 15). Summary: At 2 months post-MI, iron is intracellular, crystalline and in Fe3+ form within damaged lysosomes.

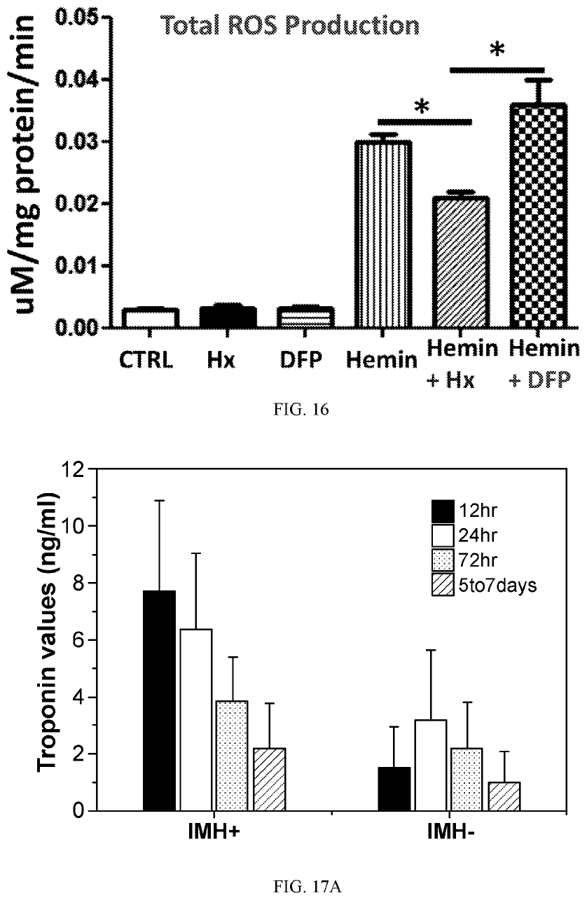

Physiochemical features of degradation products of hemorrhage are important for determining optimal therapy. An early byproduct of hemorrhage is extracellular heme (an Fe²⁺ complex), which is eventually internalized by cardiomyocytes. Applicant tested whether pretreating cardiomyocytes in culture with hemopexin (Hx), a heme scavenger, can rescue the cells from oxidative stress compared to an intracellular Fe³⁺ chelator deferiprone (DFP). Methods: Studies were performed in an cardiomyocyte culture. Total ROS following treatment with Hx, DFP, hemin, hemin+Hx, hemin+DFP were measured. Results: Treating cells with Hx or DFP did not increase total ROS compared to controls; but, hemin markedly increased ROS. Pre-treatment with Hx and then exposing to hemin significantly reduced ROS versus hemin and pre-treatment with DFP (FIG. 16). Summary: Location of hemorrhagic byproducts (intra- vs. extra-cellular) and oxidation state are critical features that need to be accounted for in the development of therapies against hMI. To develop an optimal ICT strategy against hMI we will identify the physiochemical features of hemorrhagic byproducts in rats with hMI sacrificed at different time points.

Methods: Data Collection: Spatial, temporal and biochemical features of iron in hMI and non-hMI rat tissue harvested immediately, 4 hrs, days 1, 2, 3 and wks 1, 2, 3, 4, and 8 post MI will be studied. TEM and EDS will be used to identify the cell types and organelles implicated. EDS and scanning TEM will be used to determine whether iron is free or crystalline. The oxidation state of iron will be determined using electron energy loss spectroscopy. 3D imaging of organelles will be performed to assess membranes integrity. Data Analysis & Interpretation: A byproduct of hemorrhage is heme, which is hydrophobic and easily crosses cell membranes, and results in increasing cytosolic and mitochondrial Fe2+ within myocytes in the first week of MI. Applicant correlates the extent of Fe2+ with ferroptosis (GSH depletion). Based on preliminary data, it is believed that Fe (a byproduct of Fenton reaction converting Fe2+) will co-localize with macrophages in the MI zone and accumulate in lysosomes as crystals within weeks 2 to 8. Applicant correlates the extent of iron deposition with TNF-α and IL-1β to assess the dependence between Fe3+ concentration and proinflammatory cytokine expression. Crystals in macrophages are expected to damage lysosomal membranes, which should become apparent by week 8. These findings are believed to be unique to hMIs. This will be the first study to characterize key time-dependent features of iron between hMIs and non-hMIs.

If in the acute phase of MI macrophages in the infarct periphery also accumulate iron and die, this would indicate that iron is toxic to macrophages and thereby delays the healing process due to diminished clearance in MI territory.

### Example 3. Iron Chelation Therapy to Reduce Ferroptosis and Infarct Expansion in Acute Phase and Proinflammatory Burden and Adverse Remodeling in Chronic Phase of in Hemorrhagic MI

Design and Rationale: There are no therapies to overcome the damaging effects of hMI. While, cardiac MRI (CMR) and iron chelation therapy (ICT) are clinically used to manage myocardial iron in non-ischemic heart disease, they have not been used to treat hMIs. A recent study in mice with reperfused MI showed that pre-treating with BPD (Fe2+ chelators) prior to ischemia, but not DFO (Fe3+ chelators), could reduce mitochondrial iron and reperfusion injury, but the link to hMI was not elucidated. Applicant hypothesizes that ICTs tailored to the spatio-temporal distribution, redox state, and crystallinity of iron can minimize infarct expansion in the acute phase and adverse remodeling in the chronic phase of hMI. Applicant tests whether FDA-approved iron chelators can significantly reduce the adverse effects of hMIs throughout the post reperfusion period. Positive outcome from this study will establish a causal relation between hMI, infarction expansion and adverse LV remodeling; and provide the first small animal, as well as and large animal evidence of translatability of an effective ICT strategy towards mitigating CHF post hMI.

Preliminary data: delayed deferiprone (DFP), but not EDTA, post reperfusion confers marked reduction in adverse inflammatory, compositional, structural and functional remodeling in rats with hemorrhagic myocardial infarction.

Applicant tested the therapeutic efficacy of DFP against EDTA in rats subjected to hMIs (90-min ischemia and then reperfusion, I/R). Methods: hMIs (n=15) were created and verified by echocardiography to have left ventricle ejection fraction (LVEF)<35%. Rats were treated as follows: with DFP (100 mg/kg/day, oral gavage) at 3 days post reperfusion (DFP+, n=5); placebo (PBS) treatment (n=5); or EDTA upon reperfusion (EDTA+, n=5, 40 mg/kg/day, oral gavage). All surviving animals were followed for 4 weeks after surgery, and then sacrificed and T2*-w CMR, histology and immunohistochemistry (H&E, EMT, PB, IL-113, TNF-α and MMP9) were performed. LVEF was assessed using echocardiography at baseline, week 1 and prior to sacrifice.

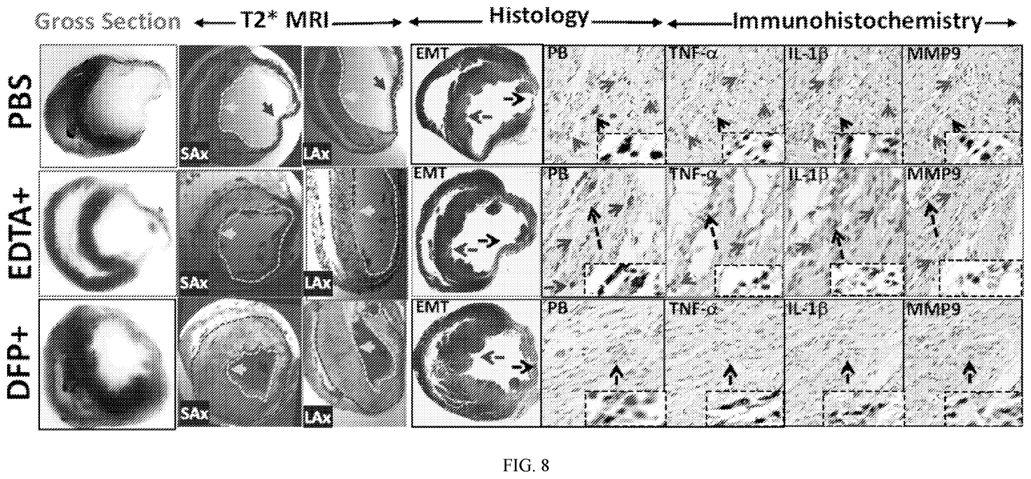

Results: In rats receiving delayed DFP, multiple indicators of protection were observed vs. EDTA-treated or placebo controls: (a) LVEF was 42±4% (DFP) vs 32±3% (EDTA) vs 30±4% (PBS); (b) little to no iron in MI zones, thicker walls (infarcted and remote) and reduced LV dilatation; and (c) reduced iron and proinflammatory markers in the scarred myocardium. See FIG. 8. Summary: (a) Delayed DFP treatment is safe and effective in reducing iron within MI and improving structural, functional, and inflammatory LV remodeling; (b) EDTA treatment does not provide cardioprotection as DFP, hence specificity of chelator is a key determinant of outcome; and (c) rat model is a viable alternative to large animal models for studying the consequences of hMI.

### Example 3-1: Prevention Arm—Reduce Infarct Expansion in Acute Phase and Mitigate Adverse Remodeling in Chronic Phase of MI

Applicant tests the efficacy of Fe²⁺ and Fe′ iron chelation therapies delivered at time points identified in Example 2 (e.g., acute phase and early chronic phase, respectively). Applicant has confirmed that key aspects of hMI (iron deposition and remodeling) between rats, large animal models and humans are similar; and absence of collaterals in rats can be used to predict myocardial injury accurately.

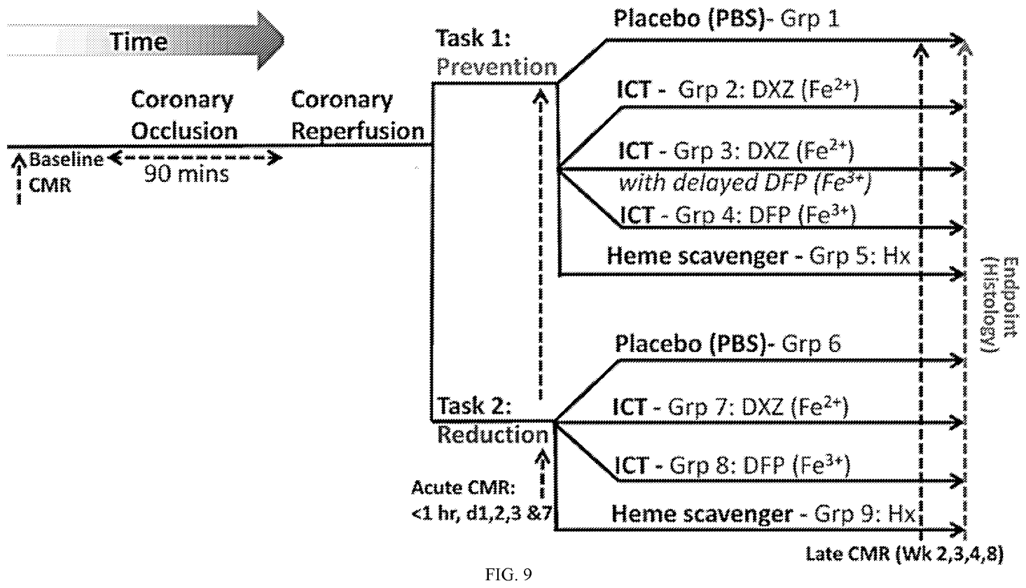

Five groups of rats (10 per group, including 20% attrition; total 63 rats) will be subjected to hMI (90-min I/R of LAD, left anterior descending coronary artery). Applicant studies the specificity of iron chelator (intracellular (DXZ, DFP) vs extracellular (EDTA), Fe²⁺ (DXZ) vs Fe³⁺ (DFP)), at clinically safe dose of 100 mg/kg, per day. Applicant compares the findings with hemopexin (Hx)-treated groups. All treatments will be initiated following reperfusion. Treatment Groups: Group 1—PBS; Group 2—DXZ for 8 weeks; Group 3—DXZ until iron crystals are formed and then DFP; Group 4—DFP; Group 5—Hx. FIG. 9 depicts the diagram. Rats will undergo cardiac MRI (CMR) (cine, T2*, and LGE) at baseline, acute MI (1-2 hrs, day 1, 2, 3, and 7), and in the late phase of MI (week 2, 3, 4 and 8). After the final cardiac MRI, hearts will be harvested, stained with TTC for infarction and undergo gene, protein and immunohistochemistry. Iron chelators will be administered at a dose of 100 mg/kg/day (oral gavage). Statistics: Statistical tools will be used to test if the mean iron content ([Fe] from T2* CMR), infarct expansion (acute phase LGE CMR), gene and protein expression. Extent of inflammation will be quantified in Definiens software, regional infarct remodeling (between day 0 and week 8) and global remodeling (change LVEF from cine CMR between day 0 and week 8, post MI) will be determined. We will identify the treatment strategy that maximally reduces infarct expansion and adverse remodeling. Mixed models with random intercepts will be used to compare the mean iron and other continuous outcomes between the various treatment groups.

Cardiac MRI (CMR): Whole-heart, ECG- and respiratory-gated 2D cine (for accurate assessment of function), T2* (accurate quantification of iron concentration), LGE (infarct size) CMR will be acquired on a 9.4T system (rat studies) or 3.0T PET/MR system (dog studies). Gene & Protein Expression and TEM of Tissue: Explanted MI sections will be used for gene and protein expression and TEM. Gene expression analysis by RT-PCR and protein expression by Western blot analysis will be performed. TEM will be used to assess morphology of cardiomyocytes. Electron Paramagnetic Resonance (EPR): Free radical production rates, along with total reactive oxygen species (ROS) and superoxide (O2.-) levels, will be measured using an EPR.

Data Interpretation: Based on the preliminary data above showing that hMIs are larger than area-at-risk and associated with extensive ROS and NADPH oxidation, it is believed that MI size of Groups 2 (DXZ ICT), 3 (DXZ and DFP) and 5 (Hx) to be much smaller than Group 1 (PBS) and Group 4 (Fe³⁺ ICT). It is possible that the MI size of Group 2 (DXZ ICT) to be even smaller than Group 5 (Hx). If this happens, this would indicate that DXZ iron chelator therapy treatment is involved in reducing Fe³⁺ not only from hemorrhage but from other sources (e.g., myoglobin stores from cardiomyocytes) as well. However, it is believed the difference to be small since the key source of Fe in acute MI is from hemorrhage. Since it is believed that DXZ will remove Fe²⁺ and DFP will remove Fe³⁺, Applicant expect to find that the optimal outcome (reduced acute MI size and LV remodeling) to be most evident in the group receiving the higher doses of DXZ in the acute phase and then receiving DFP following the formation of iron crystals. If this is not observed but Groups 2, 3 and 5 all remodeled the same (but better than Groups 1 and 4), this would indicate that removing the early byproducts of hemorrhage (extracellular heme or its intracellular Fe′ counterpart) is sufficient to limit both the acute and chronic effects of reperfusion hemorrhage. These findings would be the first in-vivo evidence that selective targeting of iron can markedly reduce the adverse effects of hemorrhage.

If limited penetration of the chelator DXZ is observed due to no reflow, a smaller iron chelator BPD may prove more effective in reducing MI size in the acute phase. Alternatively, Hx could emulate the benefits of DXZ. It is possible that based on CMR scans, [Fe] is not reduced within MI but infarct expansion is reduced; this would indicate that iron is chelated and made ineffective, though it cannot be cleared from the MI zone.

### Example 3-2: Reduction Arm—Limit Chronic Effect of hMI and Reduce Adverse Remodeling Once Iron Crystals are Formed

Applicant tests the capacity of delayed delivery of Fe³⁺ chelators to reduce adverse remodeling once iron crystals are formed, and translate the findings in a rodent model in a validated canine model of hMI.

Four groups of rats (10 per group including 20% attrition; total 50 rats) will be subjected to hMI as in example 3-1. There are two key differences between this example and example 3-1. First, treatment (PBS, ICT (DXZ and DFP) will start at the earliest time-point when iron crystals are formed (determined in Example 2; see FIG. 9). Second, CMR will be performed at wks 1, 2, 3, 4 and 8 post MI as infarct expansion is believed to be complete within 1-wk post-MI. Statistics: Iron, gene and protein expression and immunohistochemistry, structural and functional remodeling will be regressed against T2* at wk 1. One-way ANOVA will be used to determine if the change in mean values of the variables of interest between the post-treatment scans, differ between groups. Statistical power estimation is similar to Example 3-1. Data Interpretation: Based on preliminary data shown above, remodeling is believed to be better in Group 8 (DFP ICT) than all other groups. This would indicate that in the Reduction Arm only the long-term effects of Fe³⁺ can be mitigated and that the benefits of DXZ and Hx that mitigate the acute effects of hemorrhage are no longer available. Findings here can be the first to show that even a delayed ICT treatment could be beneficial for hMI patients.

It is possible that DXZ would be observed to also reduce adverse remodeling but not to the same extent as DFP. This would indicate that this effect may be mediated through the removal of residual Fe²⁺ iron, limiting conversion to Fe³⁺. Additional studies to quantify the Fe²⁺ to Fe³⁺ ratio when iron crystals are formed may be performed.

### Example 3-3: Translational Study-Evaluation of Prevention Arm in a Canine Model of hMI

Treatment Protocol & Data Collection: 4 groups of dogs (11 per group including 10% attrition; n=44) will be subjected to hMI to test the capacity of prevention arm to reduce MI expansion in the acute phase (within 72 hours (3 days) of reperfusion) and adverse remodeling in the chronic phase (week 8). Treatment Groups: Grp 1—PBS; Grp 2—DXZ for 8 weeks; Grp 3—DXZ until iron crystals are formed and then DFP; and Grp 4—DFP. LAD of dogs will be instrumented with hydraulic occluder prior to MI induction. hMIs will be created in the LAD territory by inflating the hydraulic occluder while the animal is inside the scanner. All studies will be performed in a whole-body PET/MR system. To remove the potential effects of collaterals, MI size will be normalized to the AAR determined using N-ammonia PET perfusion following LAD ligation. Data Analysis: Iron, gene and protein expression and immunohistochemistry, and remodeling parameters will be regressed against T2* at week 1. Statistical tools will be the same as in Example 3-1. Data Interpretation: Based on the close relationships Applicant has seen between rats and dogs (hemorrhage, iron deposition and inflammation), it is believed the findings would be similar to that in Example 3-1. These findings would represent the first large-animal evidence that selective targeting of iron can markedly reduce the adverse outcomes associated with hMI.

If studies in Example 2 does not provide distinct time frames in which Fe²⁺ and Fe³⁺ chelators could be tested, applicant alters the experiments to test whether outcomes associated with DXZ only, DFP only and DXZ and DFP delivered as a cocktail starting immediately after reperfusion would provide differential results. This would not be tested at the outset in order to have the opportunity to independently evaluate the time-dependent benefits of different chelators to identify an optimal therapeutic strategy. If iron is only partially cleared by DXZ and DFP, one approach applicant takes is increasing higher doses of iron chelators.

Dogs in this study will receive the same dose of iron chelator therapies as rats, though lower doses may also be efficacious due to lower metabolic rates of dogs compared to rats. Nonetheless, iron chelators at a dose of 100 mg/kg has been shown to be safe in dogs.

### Example 4-1. Infarct Expansion Studies in Subjects with Hemorrhagic or Non-Hemorrhagic MI

Myocardial infarction is a pathological process characterized by myocyte cell death precipitated by a profound reduction in blood flow to the myocardium. It is most frequently initiated by an upstream coronary artery blockage secondary to thrombosis. The severity of myocardial infarction is a function of 1) The volume of myocardium exposed to the reduction in blood flow, 2) The duration of the ischemic event, and 3) Other less well-defined factors.

Following the cessation of blood flow, myocytes and endothelial cells within the myocardial tissue swell as intracellular energy stores deplete, platelets become activated and fibrin is deposited in the ischemic capillary bed. Subsequently, apoptosis pathways are activated within injured cells and tissue architecture breaks down. The resultant damage to the microvasculature renders the restoration of normal blood flow to the tissue impossible even when the precipitating epicardial blockage is relieved.

It has been observed in both experimental models and clinical trials that the late restoration of blood flow to ischemic myocardium often results in greater infarct size than when the coronary blockage is left uncorrected. This reperfusion injury has led to guideline recommendations against late revascularization and is characterized by intramyocardial hemorrhage within the infarction zone. It remains controversial as to whether the presence of infarct associated hemorrhage contributes to myocardial injury or is a bystander phenomenon.

Advances in Cardiac MRI imaging techniques have provided greater insight into the pathophysiology of myocardial infarction as techniques to image infarct size, myocardial blood flow, microvascular obstruction, and the presence of hemorrhage have become available. We utilized these techniques to determine if differences in the pathophysiology of infarct development could be detected when comparing hemorrhagic and non-hemorrhagic infarctions.

Patients in the Study

This study was approved by our Institutional Review Board Committee, and all patients provided written informed consent. Between July 2017 and March 2019, consecutive STEMI patients undergoing primary PCI were prospectively enrolled and underwent their CMR scan at 5-8 days post PCI on a 3.0T (MAGNETOM Verio, Siemens Healthcare, Germany). The CMR scan is where cardiac cine (for function), T2* mapping (for IMH) and late-gadolinium enhancement (LGE, for MI sizing and MVO) covering the entire left ventricle were acquired. The main exclusion criteria were previous myocardial infarction (MI), ongoing arrhythmia, history of heart failure, renal insufficiency, metallic prosthetic implant, allergy to (or contraindication for) contrast agents, and claustrophobia. Among all the 70 patients initially recruited, six were excluded due to poor image quality (n=3), a previous cardiac stent (n=2), and absence of LGE (n=1). Finally, 64 STEMI patients made up the study cohort.

Proof-of-Concept Studies in Canines

Canines (n=25, 20-25 kg) were studied with surgically induced coronary stenosis. All animals were studied according to the NIH “Guide for the Care and Use of Laboratory Animals” following approval of the Institutional Animal Care and Use Committee. All animals underwent baseline scans and then subjected to (left) thoracotomy for imposing coronary stenosis studies. During left thoracotomy, one animal died, and the remaining 24 animals (n=24) were studied with 3 hours surgically induced flow-limiting stenosis of the left-anterior descending coronary artery (LAD) and subsequent reperfusion within the scanner. To introduce coronary stenosis, left lateral thoracotomy was performed as previously described and a 20 MHz Doppler probe was attached immediately distal to the first branch of the left anterior descending coronary artery (LAD) to enable measurement of coronary blood flow velocity (CBFV). An externally actuated hydraulic occluder was affixed proximal to the Doppler flow probe. Subsequently, the chest was closed.

The animals were scanned immediately after reperfusion. Animals were monitored postoperatively until they are aware of their surroundings and sternal recumbent. Following surgery, animals received routine postoperative analgesia and were monitored daily for discomfort or distress. Signs of discomfort and/or distress were defined as listlessness, failure to produce stools and/or urine, failure to eat, failure to show usual signs of mobility, and unusual physical symptoms, including redness or swelling of the surgical site. Postoperatively, before each dog awakened, Buprenex (0.1 mg/kg IM) was administered to the animal to alleviate pain and stress. This dosage was continued every 6 hours for 24-36 hours, as indicated by the comfort level of the animal. Antibiotics, Cefazolin (25 mg/kg, IV) was postoperatively administered to animals every 8 hrs for at least 24 hrs. Induction of anesthesia was with Brevital (Methohexital sodium, 11 mg/kg IV), along with pre-anesthetic tranquilizer Innovar (Fentanyl citrate 0.4 mg/ml and Droperidol 20 mg/ml). Prior to all imaging studies, animals were fasted, sedated, intubated and anesthetized with propofol (2.0-5.0 mg/kg, IV). During the imaging studies, anesthesia was maintained with a continuous infusion of propofol (0.03-0.1 mg/kg/min, IV).

Patients Troponin T Assessment

Troponin T was measured (Elecsys Troponin T; Roche) as a biochemical measure of infarct size in patients. The assay reaches a level of detection of 0.01 pg/mL and achieves <10% variation at 0.03 pg/mL, corresponding to the 99^{th} percentile of a reference population. All the patients underwent venous blood sampling for troponin measures at five occasions, that is, before PCI, less than 12 h, less than 24 h, less than 72 h, and 5 to 7 days post PCI.

Canines Troponin T Assessment

Troponin T was measured (Elecsys Troponin T; Roche) as a biochemical measure of infarct size in patients. The assay reaches a level of detection of 0.01 pg/mL and achieves <10% variation at 0.03 pg/mL, corresponding to the 99^{th} percentile of a reference population. All the patients underwent venous blood sampling for troponin measures at five occasions, that is, baseline, immediate after I/R, one day, three days, five days, and seven days post PCI.

Cardiovascular MR in Patients

Cardiac MR studies were performed within 10 days post PCI at 1.5- and 3.0T (MAGNETOM Aera and Verio, Siemens Healthcare, Erlangen, Germany) clinical MR scanner. Short-axis balanced steady-state free precession (SSFP) cine imaging, T2* maps, and late gadolinium enhancement (LGE) covering the entire heart were respectively performed.

1.5T: Typical Imaging parameters for cine-SSFP were TR/TE=2.5/1.1 ms, flip angle 80°, and GRAPPA with a parallel acceleration factor of 2. Intramyocardial hemorrhage were evaluated using T2*-maps from multi-gradient recalled acquisitions. Typical Imaging parameters were: TR=800 ms, 8 echoes with TEs=2.1, 3.9, 5.7, 7.5, 9.4, 11.2, 13, and 14.8 ms, flip angle 18°, slice thickness 8 mm, and bandwidth=814 Hz/pixel. Segmented breath-hold LGE images were acquired 10 min post-injection of contrast agent using segmented phase-sensitive inversion recovery (PSIR) reconstruction (TR/TE/TI=1 lms/3.2 ms/300 ms, flip angle 25°, bandwidth=140 Hz/pixel).

3.0T: Typical Imaging parameters for cine-SSFP were TR/TE=40.4/2.4 ms, flip angle 12°, and GRAPPA with a parallel acceleration factor of 2. T2* maps were acquired using the following imaging parameters: TR=149 ms, eight echoes with TEs=1.5, 3.8, 6.0, 8.2, 10.4, 12.6, 14.9, and 17.1 ms, flip angle 10°, slice thickness 8 mm, and bandwidth 1030 Hz/pixel. Typically used LGE imaging parameters were TR/TE=750 ms/1.6 ms, TI=300 ms, flip angle 20°, and bandwidth=465 Hz/pixel.

Cardiovascular MR in Canines

CMR was performed on eight occasions (baseline, immediate, one day, two days, three days, five days, seven days, and eight weeks post I/R) using 3T (Biograph mMR, Siemens Healthcare, Erlangen, Germany) MR system.

Late-gadolinium-enhancement (LGE) CMR: Phase-sensitive inversion recovery (PSIR) LGE acquisitions were prescribed to detect infarctions. PSIR LGE images were acquired 10 minutes after Gd-DTPA infusion (0.2 mmol/kg, Gadovist, Bayer Healthcare.), using non-selective inversion recovery preparation with GRE readout (TR/TE=3.2/1.5 ms, FA=20°, BW=586 Hz/pixel, matrix=96×192, in-plane resolution=1.3×1.3 mm2; and slice thickness=6.0 mm). A TI-scout sequence was used to find the optimal TI for nulling the healthy myocardium (240-270 ms).

Slice-matched Short-axis T2* maps were used to evaluate intramyocardial hemorrhage based on the parameters as follows: TR=240 ms, 6 TEs=3.4-18.4 ms with ΔTE=3.0 ms, flip angle 12°, and bandwidth=566 Hz/pixel. T2* maps at 3.0T were obtained (TR=20 ms, 6TEs=2.0-21.5 ms with ΔTE=3.9 ms, flip angle 10°, and bandwidth=930 Hz/pixel).

CMR Image Analysis in Patients and Canines

All CMR image analyses in both patients and canines were performed on cvi⁴² software (Circle Cardiovascular Imaging Inc., Calgary, Canada) by two experienced radiologists with at least six years of CMR experience, and a consensus was reached between readers in case of disagreement.

Myocardial infarctions (MIs) were identified based on LGE images by using a threshold setting at 5SD above the mean signal intensity of remote myocardium, and the regions of microvascular obstruction (MVO) were manually delineated as part of the MI zone. The hemorrhagic zones were defined as hypointense regions with mean signal intensity (T2*) at least 2SD below that of the reference region of interest (ROI) within MI on the T2* maps. ROIs of hemorrhage were then manually drawn at each slice around the identified myocardium, and the whole heart T2* value was then obtained. The hemorrhage volume was measured as percentage of left ventricular volume. For the non-hemorrhagic MI cases, the T2* value was acquired using the ROI of MIs copied from LGE, and the regions affected by off-resonance artifacts were manually excluded. Myocardial salvage was calculated by subtraction of percentage of infarct size from the percentage of area at risk. The myocardial salvage index was calculated by dividing the myocardial salvage area by the initial area at risk.

13N-Ammonia PET Study in Canines

All PET images were acquired using a whole-body clinical Biograph mMR (Siemens Healthcare) in 3D list mode using 13N-ammonia [100 MBq, IV bolus (30 s) followed by 10 cc saline flush] as the blood flow tracer. Before each PET scan, MR images were acquired to correct for photon attenuation. Attenuation correction was performed using 2-point Dixon MR images. PET data were obtained over 10 minutes and was started a few seconds before the 13N-ammonia injection. In group intact, images were acquired during adenosine infusion at rest. Specifically, under adenosine, PET acquisitions were prescribed after 2 min of adenosine infusion.

Quantification of Total Reduction in Perfusion Volume and Visual Scoring

The myocardial perfusion defect was measured using the rest perfusion analysis in QPET software. Total reduction in myocardial perfusion volume was derived as a fraction of the total LV myocardial volume (TRP, % LV). The extent of perfusion defect was also measured by consensus of 2 observers following guidelines of the American Society of Nuclear Cardiology. Images were scored using the 17-segment model and 5-point scoring (0, normal perfusion; 1, mild count reduction; 2, moderate count reduction; 3, severe count reduction; 4, absent uptake). Visual scores of 3 or greater were defined as the cutoff for disease.

Statistical Analysis

Continuous variables were expressed as mean±standard deviation (SD) or median and interquartile range (IQR) as appropriate. Categorical variables were reported as numbers and percentages were analysed using the Chi-squared test. The normality test of continuous variables was assessed by the Kolmogorov-Smirnov test. Differences between subgroups were compared by unpaired t-test, and continuous variables were correlated using Pearson's correlation coefficient and Spearman's correlation coefficient. Receiver operating characteristics (ROC) analysis was then performed using the mean-2SD approach as the reference standard, and comparison of ROC or independent ROC curve (AUC) was evaluated using the DeLong method. All tests were two-tailed, and a p value<0.05 was indicative of statistical significance. Statistical analyses were conducted using IBM SPSS Statistics, V.20.0 (Chicago, Ill., USA) and MedCalc version 19 (MedCalc Software, Mariakerke, Belgium).

Results

All the 64 patients had undergone successful revascularization (defined as post-PCI TIMI flow ≥2) with primary PCI within 12 h of symptoms onset (median: 300 min, range: 240-594). The demographic, angiographic, laboratory results, medication during admission, as well as CMR findings, are respectively shown in Table 2. The culprit vessel was the left anterior descending, left circumflex, and right coronary artery in 43, 9, and 14 patients, respectively. Myocardial hemorrhage was detected in 45 patients (70%). There was no significant difference in age, time from symptoms onset to revascularization, or history of angina, cardiovascular risk factors between hemorrhagic and non-hemorrhagic MI patients. More patients in the hemorrhagic MI group had pre-PCI TIMI flow 0 or 1 (P<0.001). No differences were found in post-PCI TIMI flow. All patients received the antiplatelet drug during PCI because this is standard of care at our institution.

Out of 64 STEMI patients enrolled, 48 patients underwent a 1.5-T MR, and the remaining 16 patients underwent a 3.0-T MR. On CMR at median five days (range four to nine), all patients had hyperenhancement on delayed enhancement imaging in the area subtended by the infarct-related artery. The mean infarct size on late enhancement CMR was 31.6% of LV mass. MVO was present as enhance defects on delayed enhancement CMR in 79.6% of patients (n=51), which involved a median of 1.58% of the LV mass (IQR 0.64 to 4.10). Intramyocardial hemorrhage was present in 70% (n=45) of the patients, and in these patients with IMH, it represented a median of 4.6% of LV mass (IQR 2.2 to 6.0). It was always less than the area of MVO in the corresponding post gadolinium images.

Difference of the Kinetic of Troponin Tin Relation to Hemorrhage Status in STEMI Patients

Based on the presence or absence IMH, patients were classified into two groups: nineteen patients with IMH(−), and forty-five patients with IMH(+). Patients with hemorrhage had a lower level of blood pressure and a higher proportion of TIMI flow one before PCI (p<0.001). Primary PCI produced a successful epicardial result in all 64 patients with TIMI 3 flow. The time between chest pain onset and reperfusion was similar in all three groups. (p=0.82)

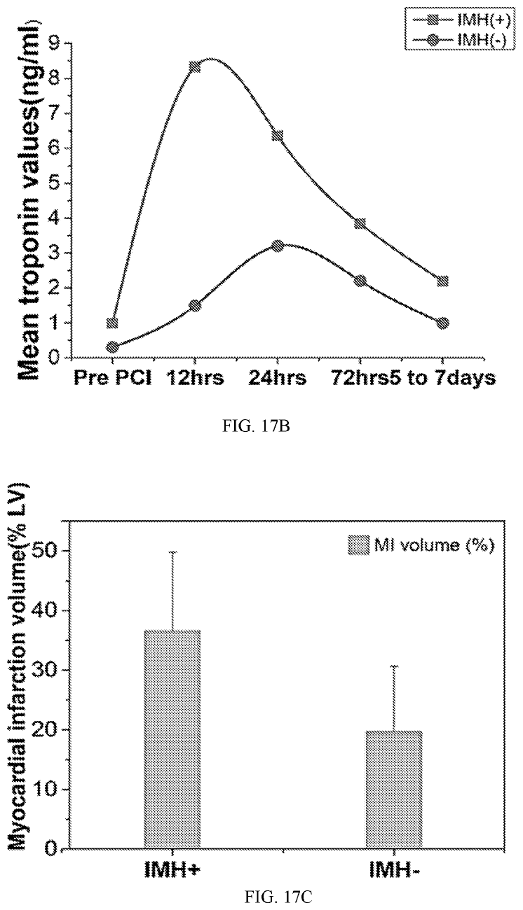

The baseline troponin T values before PCI were similar among patients in all three groups (p=0.34). However, after reperfusion, the troponin T values were significant higher in patients with IMH(+) group compared with patients IMH(−) at less than 12 h (p<0.001), 24 h (p<0.001), 72 h (p=0.001), and 5 to 7 days (p=0.002) post PCI. Consistent with the troponin data, the infarct size (percentage of left ventricular mass) assessed by CMR was also significant larger in patients with IMH₍₊₎ group compared with patients IMH₍₋₎ (36.6±13.2 vs 18.2±9.5) (Table 2, FIG. 17C). In patients with IMH, a sharp and significant increase in troponin T values was detected as early as less than 12 h post PCI. In contrast, the peak of troponin T level was presented later at 24 h post PCI with a significantly lower level in patients without IMH independent of the absence of MVO. (FIG. 17B).

TABLE 2
Patient’s baseline characteristics

| | | | No | | | | | | |
|---|---|---|---|---|---|---|---|---|---|
| | Total | | hemorrhage | | MVO without | | Hemorrhage | | |
| | cohort | | No MVO | | hemorrhage | | with MVO | | |
| | (n = 64) | | (n = 13) | | (n = 6) | | (n = 45) | | p value |
| Age | 56 ± 8 | | 56 ± 10 | | 51 ± 14 | | 57 ± 7 | | 0.27 |
| Male sex, n (%) | 57 | (89%) | 12 | (92%) | 4 | (67%) | 41 | (91%) | 0.81 |
| Body mass | 24.6 ± 2.6 | | 24.9 ± 3.3 | | 26.0 ± 3.0 | | 24.3 ± 2.4 | | 0.34 |
| index (kg/m²) | | | | | | | | | |
| Cardiovascular risk | | | | | | | | | |
| factors, n (%) | | | | | | | | | |
| Hypertension | 37 | (58%) | 9 | (69%) | 4 | (67%) | 24 | (53%) | 0.53 |
| Diabetes | 19 | (30%) | 4 | (31%) | 2 | (33%) | 13 | (29%) | 0.87 |
| Hyperlipidemia | 26 | (41%) | 5 | (38%) | 3 | (50%) | 18 | (40%) | 0.88 |
| Smoking | 39 | (61%) | 9 | (69%) | 2 | (33%) | 28 | (62%) | 0.31 |
| Heart rate (bpm) | 80 ± 12 | | 77 ± 10 | | 87 ± 17 | | 80 ± 11 | | 0.27 |
| Systolic blood | 121 ± 19 | | 131 ± 21 | | 138 ± 16 | | 116 ± 16 | | <0.005* |
| pressure (mmHg) | | | | | | | | | |
| Diastolic blood | 76 ± 13 | | 82 ± 17 | | 84 ± 16 | | 73 ± 10 | | <0.05* |
| pressure (mmHg) | | | | | | | | | |
| Symptom onset to | 5.0 | (4.0-9.9) | 6.0 | (3.5-9.5) | 4.0 | (2.8-15.3) | 4.0 | (4.0-9.8) | 0.82 |
| reperfusion (hrs), | | | | | | | | | |
| median (IQR) | | | | | | | | | |
| Number of diseased | | | | | | | | | 0.37 |
| arteries, n (%) | | | | | | | | | |
| 1 | 22 | (34%) | 6 | (46%) | 3 | (50%) | 13 | (29%) | |
| 2 | 19 | (30%) | 4 | (31%) | 0 | (0%) | 15 | (33%) | |
| 3 | 23 | (36%) | 3 | (23%) | 3 | (50%) | 17 | (38%) | |
| Infarct-related artery, | | | | | | | | | 0.10 |
| n (%) | | | | | | | | | |
| Left anterior | 41 | (64%) | 8 | (62%) | 3 | (50%) | 30 | (67%) | |
| descending | | | | | | | | | |
| Left circumflex | 9 | (14%) | 4 | (31%) | 2 | (33%) | 3 | (7%) | |
| Right coronary | 14 | (22%) | 1 | (8%) | 1 | (17%) | 12 | (27%) | |
| artery | | | | | | | | | |
| TIMI flow grade | | | | | | | | | <0.05* |
| before PCI, n (%) | | | | | | | | | |
| 0-1 | 57 | (89%) | 9 | (69%) | 5 | (83%) | 43 | (96%) | |
| 2-3 | 7 | (11%) | 4 | (31%) | 1 | (17%) | 2 | (4%) | |
| TIMI flow grade 3 | 64 | | 13 | (100%) | 6 | (100%) | 45 | (100%) | / |
| after PCI, n (%) | | | | | | | | | |
| Blood results less | | | | | | | | | |
| than 24 h post PCI | | | | | | | | | |
| Troponin T (ng/ml) | 5.6 ± 3.0 | | 3.1 ± 2.3 | | 3.4 ± 3.0 | | 6.4 ± 2.7 | | <0.001* |
| CK-MB (U/L) | 142 | (90-228) | 105 | (68-214) | 132 | (58-224) | 144 | (96-221) | 0.50 |
| NT-pro BNP (pg/ml) | 918 | (608-1676) | 585 | (261-1060) | 948 | (382-3400) | 1026 | (668-2049) | <0.05* |
| hs-CRP (mg/L) | 8.2 | (4.2-19.2) | 5.6 | (1.5-10.4) | 4.8 | (3.2-31.4) | 11.3 | (5.0-34.6) | <0.02* |
| Laboratory test | | | | | | | | | |
| TC (mmol/L) | 4.7 ± 1.3 | | 4.7 ± 1.4 | | 4.3 ± 1.2 | | 4.7 ± 1.2 | | 0.87 |
| TG (mmol/L) | 1.7 ± 1.0 | | 1.9 ± 1.2 | | 1.5 ± 0.5 | | 1.7 ± 1.0 | | 0.72 |
| LDL-C (mmol/L) | 2.8 ± 1.1 | | 2.9 ± 1.0 | | 2.7 ± 1.1 | | 2.9 ± 1.1 | | 0.97 |
| HDL-C (mmol/L) | 1.1 ± 1.1 | | 1.0 ± 0.3 | | 0.9 ± 0.1 | | 1.1 ± 0.3 | | 0.37 |
| BUN (mmol/L) | 4.9 ± 1.5 | | 4.5 ± 1.2 | | 4.2 ± 1.4 | | 5.2 ± 1.6 | | 0.14 |
| Cr (μmol/L) | 84 ± 19 | | 84 ± 15 | | 74 ± 21 | | 84 ± 20 | | 0.47 |
| Uric acid (μmol/L) | 364 ± 94 | | 351 ± 92 | | 410 ± 91 | | 362 ± 91 | | 0.41 |
| eGFR | 92 | (76-100) | 89 | (79-101) | 97 | (80-115) | 92 | (77-100) | 0.55 |
| (ml/min/1.73), | | | | | | | | | |
| median (IQR) | | | | | | | | | |
| Glucose (mmol/L), | 6.6 | (5.8-8.0) | 6.0 | (5.4-8.0) | 6.6 | (5.5-7.6) | 7.1 | (6.2-8.5) | 0.22 |
| median (IQR) | | | | | | | | | |
| Medication during | | | | | | | | | |
| admission, n (%) | | | | | | | | | |
| Antiplatelet drug | 64 | (100%) | 13 | (100%) | 6 | (100%) | 45 | (100%) | / |
| Beta blocker | 61 | (95%) | 12 | (92%) | 6 | (100%) | 43 | (96%) | 0.76 |
| ACEI | 53 | (83%) | 11 | (85%) | 4 | (67%) | 38 | (84%) | 0.55 |
| ARB | 15 | (23%) | 5 | (38%) | 2 | (33%) | 8 | (18%) | 0.25 |
| CCB | 5 | (8%) | 0 | (0%) | 1 | (17%) | 4 | (9%) | 0.40 |
| Diuretic | 26 | (41%) | 2 | (15%) | 4 | (67%) | 20 | (44%) | 0.07 |
| Statin | 64 | (100%) | 13 | (100%) | 6 | (100%) | 45 | (100%) | / |
| Amiodarone | 6 | (13%) | 0 | (0%) | 0 | (0%) | 6 | (13%) | 0.25 |
| Nitrate | 56 | (88%) | 13 | (100%) | 6 | (100%) | 37 | (82%) | 0.15 |
| Cardiac magnetic resonance findings | | | | | | | | | |
| Hemorrhagic | | | 0 | | 0 | | 4.61 | (2.18-5.96) | / |
| volume (% LV) | | | | | | | | | |
| Hemorrhagic T2* | 26.5 ± 14.7 | | 48.8 ± 10.1 | | 37.7 ± 12.1 | | 18.5 ± 5.7 | | <0.001* |
| value (ms) | | | | | | | | | |
| Infarct volume | 31.6 ± 14.7 | | 18.2 ± 9.5 | | 23.0 ± 13.9 | | 36.6 ± 13.2 | | <0.001* |
| (% LV) | | | | | | | | | |
| MVO volume | 1.58 | (0.64-4.10) | 0 | | 0.93 | (0.64-2.37) | 2.68 | (1.42-4.48) | <0.001* |
| (% LV) | | | | | | | | | |

TC indicates total cholesterol;
TG, triglyceride;
LDL-C, low density lipoprotein cholesterol;
HDL-C, high density lipoprotein cholesterol;
BUN, blood urea nitrogen;
Cr, Creatinine.
Data are reported as mean ± SD, median (IQR), or n (%) as appropriate.

Morphological and Infarct Parameters at Baseline in Canines

Of the 25 canines that enrolled the study, four were excluded: one died during the reperfusion phase, one at least one infarction was absent, 2 with pretreatment. Of the remaining 21 dogs, four were killed at three days, and the other 17 canines were all kept at least eight weeks. Serial Troponin measurements were available in all 21 canines.

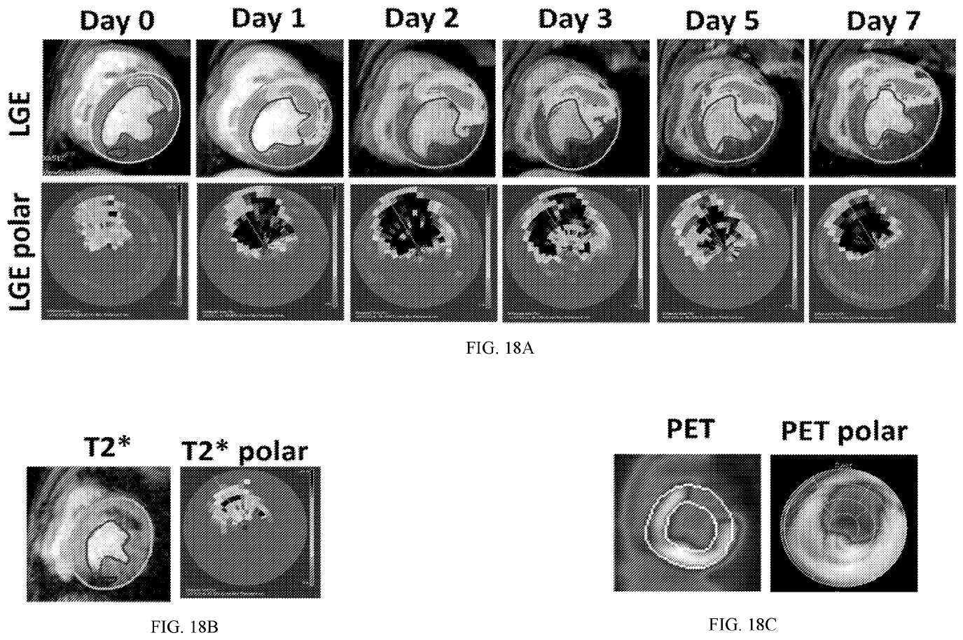

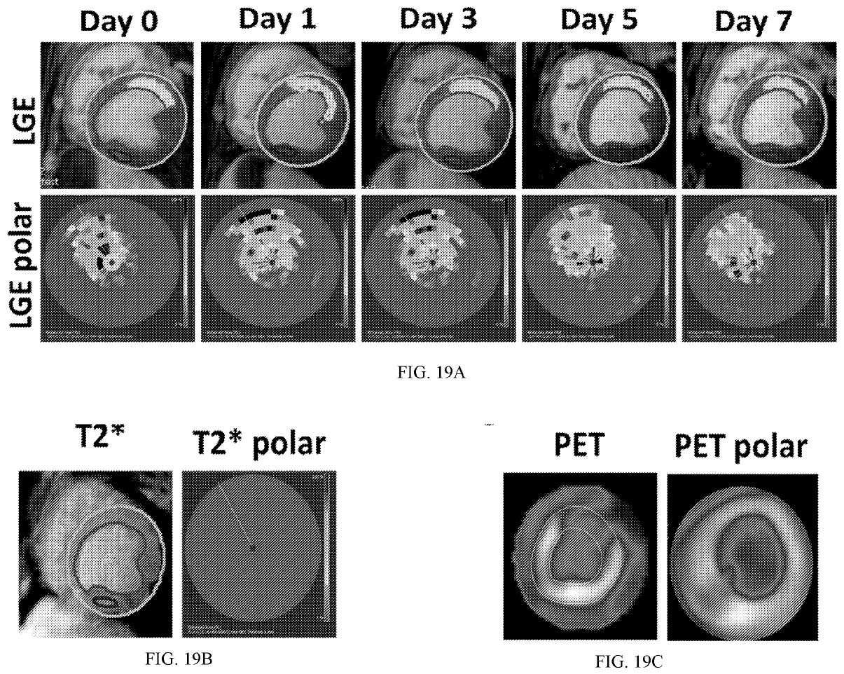

Out of twenty-one animals were available for imaging, hemorrhage was evident in 12 animals and appeared hypointense on T2* CMR. No hemorrhage was evident in the other animals (n=9). LGE images from the same animals with hemorrhage and without hemorrhage in these serial scans (immediate, one day, two days, three days, five days and seven days and eight weeks post I/R) of MI, and corresponding noncontrast-enhanced T2*, PET images are shown in FIGS. 18A-18C and 19A-19C, respectively. The area at risk assessed by PET and the ratio of infarct size to the area at risk were similar in the hemorrhagic compared with the non-hemorrhagic group on day 0(47.6±6.3 vs. 45.4±9.7, p=0.590; 45.1±11.2 vs. 42.5.0±11.8, v0.644). MVO was present in all animals with hemorrhagic MI and in 2 (22%) of animals with non-hemorrhagic MI.

Temporal Evolution of Infarct Size in Relation to Hemorrhage Status

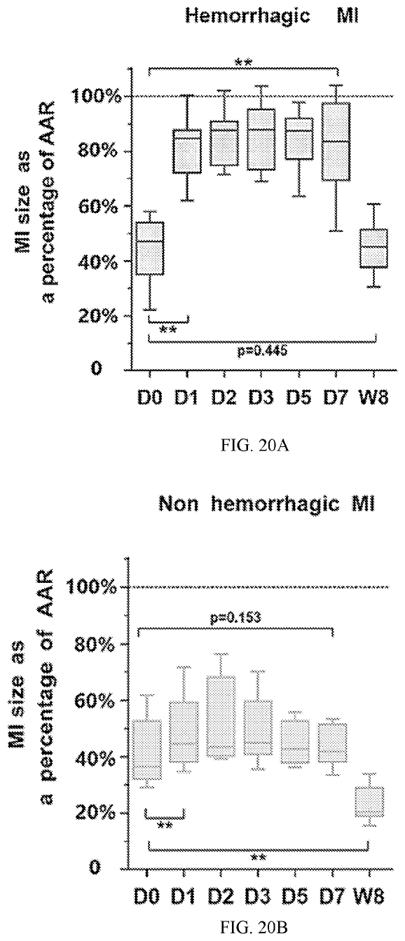

Hemorrhagic MIs were visualized as hypointense regions on T2* maps in acute and chronic phases 3.0-T (FIG. 19A) within the MI territories identified on LGE CMR. The same animals followed 24 h post I/R subsequently increased compared with the MI size of immediately post I/R (18.7±5.7 vs. 22.0±5.4, p<0.01) and followed by a stabilized status until seven days (p=0.153) (FIG. 18A and FIG. 20A). Even with a decreased trend of MI size, the infarcted regions of the same animals followed to the chronic phase of MI (8 weeks) were still visualized much larger than the animals without hemorrhage.

Non-hemorrhagic MIs were identified as those MIs without T2* loss in the acute phase (FIG. 19C). Compared to LGE images immediately post I/R, the same animals followed one day post I/R show slightly increase of MI size (21.5±5.8 vs. 39.0±6.2, p<0.001) and by a subsequent decrease over the following days to reach values on day seven similar to those observed at immediate post I/R (p=0.153) (FIG. 18A and FIG. 20A).

Correlation of MI Expansion with a Post I/R Myocardial Hemorrhage Size

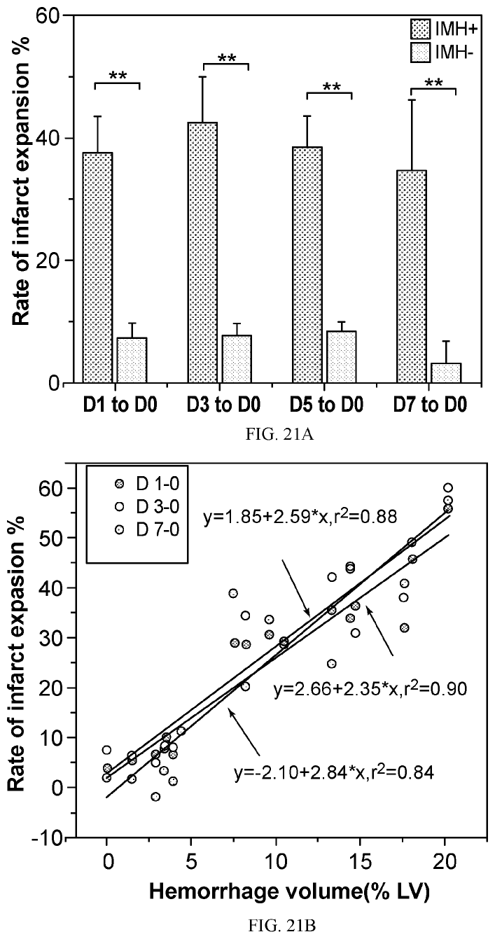

Canines with hemorrhagic MI had significantly larger expand index than non-hemorrhagic MI at both one day and 7 days (all p<0.001) (FIG. 21A). Percentage infarct transmurality was substantially higher in the hemorrhagic group both at baseline and at eight weeks. Myocardial hemorrhage size ranged from 7.5 to 20 of LV % (median: 13.2%). FIG. 21B shows the relationship between hemorrhage and expand index at day 1 minus day 0 according to the ratio of infarct size to AAR. The strong linear correlation was noted between the myocardial hemorrhage size (LV %) and the MI expand index (%) (r=0.8984; P<0.001).

Temporal Evolution of Myocardial Salvage in Relation to Hemorrhage Status

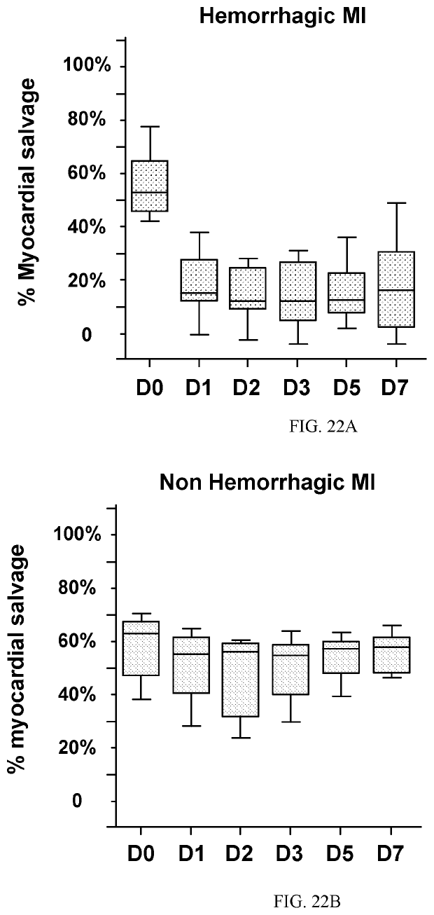

Myocardial salvage calculating by LGE based myocardial infarct size and PET based AAR was similar at immediate post I/R on both hemorrhage MI and non-hemorrhagic MI groups (52.2±19.4 vs. 57.4±11.8, p=0.538). Besides, there is no significant reduction of the amount of salvageable myocardium with the timing of serial CMR post I/R until seven days in non-hemorrhagic MI group (50.1±12.7 vs. 57.4±11.8, p=0.538). However, there were significantly less salvageable myocardium in same canines with hemorrhage MI from 1 day to 7 days than immediate post I/R (17.36±12.9 vs. 52.2±19.4, p<0.001) (FIGS. 22A and 22B).

### Example 4-2. Intramyocardial Hemorrhage and the “Wave-Front” of Reperfusion Injury

Overall

Reperfusion therapy in ST-elevation MI is life-saving; however, its benefits can be paradoxically diminished from further increase in MI size even after the blood flow to the epicardial coronary artery is restored. This phenomenon, often referred to as reperfusion injury, has been associated with microvascular injury as well as incremental damage to the myocardium. To date, experimental therapies to mitigate reperfusion damage have not been successful, leading to calls for improved understanding of reperfusion injury. Intramyocardial hemorrhage (IMH), a potential consequence of reperfusion is associated with larger MIs, but whether it contributes to infarct expansion is unknown. We hypothesized that hemorrhagic transformation of reperfused MI catalyzes time-dependent infarct expansion and is a key determinant of final MI size.

We studied cardiac troponin kinetics (cTn) of ST-elevation MI patients (n=70) treated with primary percutaneous coronary intervention (PCI) identified to be hemorrhagic (70%) or non-hemorrhagic MI, before PCI and at multiple time points after PCI (12 h, 24 h, 48 h, 72 h and 5-7 days). To validate our clinical findings, we performed ischemia-reperfusion studies in canines (n=25) with (60%) and without IMH, and serially followed cTn levels and sized MIs based on time-lapse imaging following reperfusion (1 h, 24 h, 28 h, 72 h, 5 and 7 days).

In patients with IMH, cTn rose rapidly following reperfusion and peaked earlier and was higher than in patients without IMH (8±4 ng/ml (IMH+, 12 h) vs 2±2 ng/ml 24 h), p<0.01), paralleling the findings in animals. Time-lapse imaging showed that in hemorrhagic animals, reperfusion led to rapid and expansive myocardial necrosis within the area-at-risk (% LV/AAR), compared to non-hemorrhagic animals within the first 24 h (35±5% vs. 8±2%, p<0.001); and that the rate of MI expansion following reperfusion was dependent on time after reperfusion (change in MI size between IMH+ IMH− groups, p<0.001 at 24 h but p>0.5 beyond 24 h) and hemorrhage volume (r²=0.90, p<0.001 at 24 h). Further, infarct expansion in animals after reperfusion followed a wave-front pattern with epicardial involvement only in the presence of hemorrhage (IMH+: 52.7±9.9% (<1 h) to 77.9±11.1% at (˜24 h), p<0.01, compared to IMH 55.6±3.4% (<1 h) vs. 59.2±1.7% (˜24 h), p=0.284), consistent with final infarct characteristics in patients. Notably, while myocardial salvage normalized to area-at-risk was not different at 1-h post reperfusion in animals with and without hemorrhage (47.6±6.3% (IMIH+) vs. 45.4±9.7% (IMH−), p=0.59), a marked loss in salvageable myocardium was evident within the first 72-h after reperfusion only in the presence of hemorrhage (20±10% (IMH+) vs 50±10% (IMH−)).

Myocardial hemorrhage is a determinant of final infarct size. It can drive a substantial loss of salvageable myocardium following reperfusion and compromise the expected benefits of reperfusion. Our findings support the notion that if hemorrhage can be avoided/reduced or its effects are mitigated following reperfusion, reperfusion therapy can confer major additional clinical benefit.

Details

Studies have shown that reperfusion can result in infarct zones with or without obstructed microvessels (microvascular obstruction, MVO). MVOs have been shown to expand following reperfusion and their expansion has been shown to parallel myocardial necrosis following reperfusion injury. Studies have also shown that regions of MVO may also be accompanied by microvascular destruction resulting in intramyocardial hemorrhage (IMH). To date however, whether IMH plays a role in infarct expansion has not been explored despite the observation that large MIs are accompanied by IMH. It is known that while red blood cells serve a vital role in oxygen delivery, their externalization into the interstitial space can be detrimental. First, following reperfusion IMH can exacerbate microvascular compromise and extend zone of hypoxia. Second, the hemolysis of red blood cells in the extracellular environment can expose cardiomyocytes to heme, an iron-binding component of hemoglobin that is cytotoxic. Thus, both effects emanating from the introduction of IMH may drive expansion of infarction zone. Based on this premise, we hypothesized that hemorrhagic transformation of reperfused MI catalyzes a time-dependent expansion of the MI zone, which contributes to a larger final MI size compared to infarctions without hemorrhage, independent of MVO.

We investigated our hypothesis in ST-elevation MI patients using repeat sampling of cardiac biomarkers pre- and post-reperfusion with patients stratified as hemorrhagic or non-hemorrhagic on the basis of magnetic resonance imaging (MRI). To validate our findings in the myocardium, we used a canine model of ischemia-reperfusion injury. Along with cardiac biomarkers, we used a clinical dual-modality system (an integrated positron-emission tomography (PET) system simultaneously registered with MRI) to perform a non-invasive time-lapse imaging study using each animal as their own control. This allowed us to use gold-standard imaging approaches to quantify the area at risk, MI size, MVO and IMH and to assess the temporal evolution of myocardial necrosis in the super-acute, sub-acute and chronic phases of reperfused infarctions with and without IMH.

Patients (n=70) undergoing primary percutaneous coronary intervention (PCI) for ST-elevation MI (STEMI) were recruited to the study. Patient studies targeted cardiac troponin kinetics to index cardiomyocyte necrosis in hemorrhagic and non-hemorrhagic MIs prior to PCI and also at multiple time points post PCI. Presence of IMH was determined on the basis of cardiac magnetic resonance imaging (CMR) exams performed on 5-7 days post PCI. Blood samples were collected in patients before and after PCI. Given the difficulties in imaging STEMI patients repeatedly, especially within the first 72 hours post PCI, large animals (n=25) with and without hemorrhagic MIs were also studied. Animal studies were performed to gather insight into the findings in patients with the specific goal of relating cardiac troponin kinetics in animals with and without hemorrhage to time-dependent changes in MI size. These studies were performed in a dual-modality clinical positron emission tomography (PET)/MRI system to assess area-at-risk based on ¹³N-ammonia PET during ischemia and infarct size based on CMR post reperfusion. This allowed us to determine the AAR with a gold-standard approach, rather than other methods (such as T2-based CMR) which remain to be widely accepted. Additional follow-up CMR studies were performed to track MI size. Blood samples were collected from animals at time points corresponding to blood draws in patients to assess the congruence in cardiac troponin kinetics.

Blood Markers and Non-Invasive Cardiovascular Imaging for Serial Assessment of Myocardial Damage

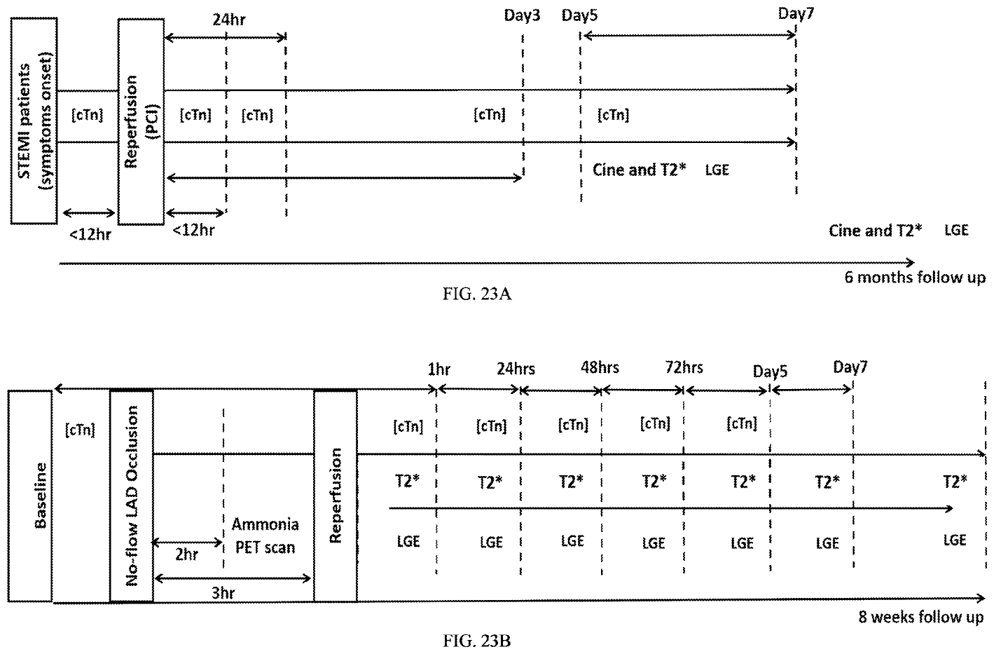

Patients in the Study are described as that in Example 4-1. A flow chart outlining the key time points at which blood sampling and CMR studies were performed is provided in FIG. 23A. Cardiac MRI studies were performed on a 3.0T MRI systems (MAGNETOM Verio, Siemens Healthcare, Erlangen, Germany) 5 to 8 days and 6-8 months post PCI. ort-axis balanced steady-state free precession (SSFP) cine imaging, T2* maps, and late gadolinium enhancement (LGE) covering the entire heart were performed. Typical Imaging parameters for cine-FLASH were TR/TE=40.4/2.4 ms, flip angle 12°, and GRAPPA with a parallel acceleration factor of 2. T2* maps were acquired using the following imaging parameters: TR=149 ms, eight echoes with TEs=1.5, 3.8, 6.0, 8.2, 10.4, 12.6, 14.9, and 17.1 ms, flip angle 10°, slice thickness 8 mm, and bandwidth 1030 Hz/pixel. Typically used LGE imaging parameters were TR/TE=750 ms/1.6 ms, TI=300 ms, flip angle 20°, and bandwidth=465 Hz/pixel.

Animal Study is described as that in Example 4-1. Additional details outlining the surgical procedure include the following. To introduce coronary occlusion, left lateral thoracotomy was performed as previously described and an externally actuated occluder was affixed immediately distal to the first branch of the left anterior descending coronary artery (LAD) to ensure no-flow ischemia. Subsequently, the chest was closed and the animals were recovered. During recovery, dogs were monitored postoperatively until they are aware of their surroundings and sternal recumbent. The animals received routine postoperative analgesia and were monitored daily for discomfort or distress after the surgery and before imaging. Signs of discomfort and/or distress were defined as listlessness, failure to produce stools and/or urine, failure to eat, failure to show usual signs of mobility, and unusual physical symptoms, including redness or swelling of the surgical site. Postoperatively, before each dog awakened, Buprenex (0.1 mg/kg IM) was administered to the animal to alleviate pain and stress. This dosage was continued every 6 hours for 24-36 hours, as indicated by the comfort level of the animal. Antibiotics, Cefazolin (25 mg/kg, IV) was postoperatively administered to animals every 8 hrs for at least 24 hrs. Induction of anesthesia was with Brevital (Methohexital sodium, 11 mg/kg IV), along with pre-anesthetic tranquilizer Innovar (Fentanyl citrate 0.4 mg/ml and Droperidol 20 mg/ml). Prior to all imaging studies, animals were fasted, sedated, intubated and anesthetized with propofol (2.0-5.0 mg/kg, IV). During the imaging studies, anesthesia was maintained with a continuous infusion of propofol (0.03-0.1 mg/kg/min, IV). The timing of imaging is illustrated in FIG. 23B. During the first imaging section, baseline images were acquired and was followed by the induction of a no-flow LAD occlusion. Ammonia PET images were acquired two hours into the no-flow LAD occlusion to ascertain the AAR. The LAD occlusion was maintained for three hours and was followed by gently releasing the LAD occlusion to re-establish blood flow. Subsequently, CMR images were acquired post reperfusion. Follow-up CMR scans in the animals were performed within 24 hrs, 48 hrs, 72 hrs, day 5, day 7 and 8 weeks post reperfusion.

Imaging studies were performed on a PET/MR system operating at 3.0T (Biograph mMR, Siemens Healthcare, Erlangen, Germany). ¹³N-ammonia PET images were acquired at least 2-hrs after the induction of ischemia (and prior to reperfusion) to determine the area-at-risk based on myocardial blood flow deficit. Following 3-hrs of no-flow ischemia, LAD was reperfused and CMR scans similar to the human patients studies (cine, T2* and LGE) were performed to assess cardiac function, IMH, MI size and MVO. Venous blood sampling and CMR studies were performed at baseline, within 1 hr, 24 h, 48 h, 72 h, 5 days and 7 days, post reperfusion. At 8-weeks post reperfusion, CMR images were acquired again to characterize chronic state of the injury. CMR was performed on eight occasions (baseline, immediate post ischemia, one day, two days, three days, five days, seven days, and eight weeks post reperfusion) using a 3T scanner (Biograph mMR, Siemens Healthcare, Erlangen, Germany). Late-gadolinium-enhancement (LGE) CMR: Phase-sensitive inversion recovery (PSIR) LGE acquisitions were prescribed to detect infarctions. PSIR LGE images were acquired 10 minutes after Gd-DTPA infusion (0.2 mmol/kg, Gadovist, Bayer Healthcare.), using non-selective inversion recovery preparation with GRE readout (TR/TE=3.2/1.5 ms, FA=20°, BW=586 Hz/pixel, matrix=96×192, in-plane resolution=1.3×1.3 mm²; and slice thickness=6.0 mm). A TI-scout sequence was used to find the optimal TI for nulling the healthy myocardium (240-270 ms). Slice-matched short-axis T2* maps were used to evaluate intramyocardial hemorrhage based on the parameters as follows: TR=240 ms, 6 TEs=3.4-18.4 ms with ΔTE=3.0 ms, flip angle 12°, and bandwidth=566 Hz/pixel. T2* maps at 3.0T were obtained (TR=20 ms, 6TEs=2.0-21.5 ms with ΔTE=3.9 ms, flip angle 10°, and bandwidth=930 Hz/pixel). All PET images were acquired 2 hours after the occlusion of the LAD artery using a whole-body clinical Biograph mMR (Siemens Healthcare). Data was collected 3D list mode using 13N-ammonia [100 MBq, IV bolus (30 s) followed by 10 cc saline flush] as the blood flow tracer. Before each PET scan, MR images were acquired to correct for photon attenuation using 2-point Dixon MR images as previously described. PET data were obtained over 10 minutes and was started a few seconds before the 13N-ammonia injection.

Cardiac troponin concentrations ([cTn]) were measured from serum samples (Canine Ultra-sensitive Cardiac Troponin-I ELISA, Cat #-CTNI-4-US, Life Diagnostics, PA, USA) using the manufacturer recommended protocol.

CMR Image Analysis is described as that in Example 4-1. Regions of MVO were manually delineated as those parts of the MI zone that were hypointense on LGE. IMH volume was measured on each slice positive for MI, summed and reported as percentage of LV volume. Both patients and animals were identified to have had a hemorrhagic MI if T2* maps were positive for IMH with IMH volume >5% of MI size. Subjects positive for IMH were labeled as IMH+, otherwise IMH−. Rate of MI expansion was calculated as the change in MI size per day normalized by PET area-at-risk. Infarct transmurality was determined as the percentage extent of the infarct along 100 equally spaced chords on each slice. Mean transmurality was obtained by averaging the infarct transmurality across all the chords that have >1% scar extent. The wall thickness of enhanced tissue and that of nonenhanced tissue were measured along 100 chords that were equally distributed along the circumference of the LV on the LGE CMR. The mean thickness of infarct myocardium was determined by averaging the measurements in each enhanced segment.

PET: Area-at-risk (AAR) as determined as the territory of total myocardial perfusion defect on rest perfusion images during complete occlusion of the LAD using QPET software. Total reduction in myocardial perfusion volume was derived as a fraction of the total LV myocardial volume (TRP, % LV). The extent of perfusion defect was also measured by two experienced reviewers in consensus based on the guidelines of the American Society of Nuclear Cardiology. The volume of the perfusion defect under complete LAD occlusion was defined as the AAR. Myocardial salvage was determined as the difference in total perfusion volume and volume of infarction (based on LGE), which is normalized to the total volume of myocardium, and reported as a percentage. The myocardial salvage index was calculated by dividing the myocardial salvage area by the AAR. Given that images were acquired in a dual modality PET/MR system, there was near-perfect registration between PET and MR images.

Statistical analysis is described as that in Example 4-1.

Results

We prospectively analyzed a cohort of clinical infarcts stratified by CMR into hemorrhagic and non-hemorrhagic MI types. 70 patients presenting to a large tertiary care hospital with STEMI and managed with successful mechanical revascularization (defined as post-PCI TIMI flow >2) within 12 h of symptoms onset (median: 300 min, range: 240-594 min) were recruited. 64 patients were included in the study; six patients were excluded on the basis of CMR due to poor image quality (n=3), absence of LGE (n=1) and previous cardiac stent (n=2). Baseline characteristics of the population, discriminated on the basis of hemorrhagic vs non-hemorrhagic STEMI, are shown in Table 3. The culprit vessel was identified as the left anterior descending (41 patients), left circumflex (9 patients), and right coronary artery (14 patients). Based on T2* CMR, myocardial hemorrhage was detected in 45 of the 64 patients (70%). There was no significant difference in patient age, time from symptom onset to revascularization, history of angina, or cardiovascular risk factors identified between the IMH+ and IMH− groups. No differences were found in post-PCI flow grade. All patients received dual antiplatelet therapy at time of PCI. There was no significant difference found in the time to reperfusion between the IMH+ and IMH− groups (P=0.97; Table 3). Patients with IMH did have lower blood pressure (p<0.05), were more likely to show reduced left-ventricular ejection fraction (LVEF, p<0.001), and a higher proportion demonstrated reduced TIMI flow before PCI (TIMI flow 0 or 1; P<0.001) compared to patients without IMH.

TABLE 3
Baseline characteristics of STEMI Patients.

| | Total cohort | | No hemorrhage | | Hemorrhage | | |
|---|---|---|---|---|---|---|---|
| | (n = 64) | | (n = 19) | | (n = 45) | | p value |
| Age | 56 ± 8 | | 55 ± 11 | | 57 ± 7 | | 0.43 |
| Male sex, n (%) | 57 | (89%) | 16 | (84%) | 41 | (91%) | 0.42 |
| Body mass index (kg/m²) | 24.6 ± 2.6 | | 25.2 ± 3.2 | | 24.3 ± 2.4 | | 0.23 |
| Cardiovascular risk factors, n (%) | | | | | | | |
| Hypertension | 37 | (58%) | 13 | (68%) | 24 | (53%) | 0.26 |
| Diabetes | 19 | (30%) | 6 | (32%) | 13 | (29%) | 0.83 |
| Hyperlipidemia | 26 | (41%) | 8 | (42%) | 18 | (40%) | 0.88 |
| Smoking | 39 | (61%) | 11 | (58%) | 28 | (62%) | 0.75 |
| Heart rate (bpm) | 80 ± 12 | | 80 ± 13 | | 80 ± 11 | | 0.80 |
| Systolic blood pressure | 121 ± 19 | | 133 ± 20 | | 116 ± 16 | | <0.005* |
| (mmHg) | | | | | | | |
| Diastolic blood pressure | 76 ± 13 | | 83 ± 16 | | 73 ± 10 | | <0.05* |
| (mmHg) | | | | | | | |
| Symptom onset to reperfusion | 5.0 | (4.0-9.9) | 5.0 | (3.0-10.0) | 4.0 | (4.0-9.8) | 0.97 |
| (hrs), median (IQR) | | | | | | | |
| Number of diseased arteries, n (%) | | | | | | | 0.34 |
| 1 | 22 | (34%) | 9 | (47%) | 13 | (29%) | |
| 2 | 19 | (30%) | 4 | (21%) | 15 | (33%) | |
| 3 | 23 | (36%) | 6 | (32%) | 17 | (38%) | |
| Infarct-related artery, n (%) | | | | | | | <0.05* |
| Left anterior descending | 41 | (64%) | 11 | (58%) | 30 | (67%) | |
| Left circumflex | 9 | (14%) | 6 | (32%) | 3 | (7%) | |
| Right coronary artery | 14 | (22%) | 2 | (11%) | 12 | (27%) | |
| TIMI flow grade before PCI, n (%) | | | | | | | <0.05* |
| 0-1 | 57 | (89%) | 14 | (74%) | 43 | (96%) | |
| 2-3 | 7 | (11%) | 5 | (26%) | 2 | (4%) | |
| TIMI flow grade 3 after PCI, | 64 | | 19 | (100%) | 45 | (100%) | / |
| n (%) | | | | | | | |
| Blood results less | | | | | | | |
| than 24 h post PCI | | | | | | | |
| Troponin T (ng/ml) | 5.6 ± 3.0 | | 3.2 ± 2.4 | | 6.4 ± 2.7 | | <0.001* |
| CK-MB (U/L) | 142 | (90-228) | 105 | (67-197) | 144 | (96-221) | 0.26 |
| NT-proBNP (pg/ml) | 918 | (608-1676) | 704 | (320-1218) | 1026 | (668-2049) | <0.05* |
| hs-CRP (mg/L) | 8.2 | (4.2-19.2) | 4.9 | (1.9-8.5) | 11.3 | (5.0-34.6) | <0.01* |
| Laboratory test | | | | | | | |
| TC (mmol/L) | 4.7 ± 1.3 | | 4.6 ± 1.3 | | 4.7 ± 1.2 | | 0.86 |
| TG (mmol/L) | 1.7 ± 1.0 | | 1.8 ± 1.1 | | 1.7 ± 1.0 | | 0.61 |
| LDL-C (mmol/L) | 2.8 ± 1.1 | | 2.8 ± 1.0 | | 2.9 ± 1.1 | | 0.94 |
| HDL-C (mmol/L) | 1.1 ± 0.3 | | 1.0 ± 0.2 | | 1.1 ± 0.3 | | 0.18 |
| BUN (mmol/L) | 4.9 ± 1.5 | | 4.4 ± 1.2 | | 5.2 ± 1.6 | | 0.05* |
| Cr (μmol/L) | 84 ± 19 | | 81 ± 17 | | 84 ± 20 | | 0.53 |
| Uric acid (μmol/L) | 364 ± 94 | | 369 ± 93 | | 362 ± 91 | | 0.78 |
| eGFR (ml/min/1.73), median | 92 | (76-100) | 95 | (82-101) | 92 | (77-100) | 0.43 |
| (IQR) | | | | | | | |
| Glucose (mmol/L), median | 6.6 | (5.8-8.0) | 6.0 | (5.5-7.7) | 7.1 | (6.2-8.5) | 0.08 |
| (IQR) | | | | | | | |
| Medication during admission, n (%) | | | | | | | |
| Dual antiplatelet therapy | 64 | (100%) | 19 | (100%) | 45 | (100%) | / |
| Beta blocker | 61 | (95%) | 18 | (95%) | 43 | (96%) | 1.00 |
| ACEI | 53 | (83%) | 15 | (79%) | 38 | (84%) | 0.72 |
| ARB | 15 | (23%) | 7 | (37%) | 8 | (18%) | 0.12 |
| CCB | 5 | (8%) | 1 | (5%) | 4 | (9%) | 1.00 |
| Diuretic | 26 | (41%) | 6 | (32%) | 20 | (44%) | 0.34 |
| Statin | 64 | (100%) | 19 | (100%) | 45 | (100%) | / |
| Amiodarone | 6 | (13%) | 0 | (0%) | 6 | (13%) | 0.17 |
| Nitrate | 56 | (88%) | 19 | (100%) | 37 | (82%) | 0.09 |

TC indicates total cholesterol;
TG, triglyceride;
LDL-C, low density lipoprotein cholesterol;
HDL-C, high density lipoprotein cholesterol;
BUN, blood urea nitrogen;
Cr, Creatinine.
Data as mean ± SD, median (IQR), or n (%).

Reperfused STEMI Patients with Intramyocardial Hemorrhage have Larger MIs

Acute phase CMR was performed at a median of five days post PCI (range four to nine days). All patients had hyperenhancement on LGE CMR in the area subtended by the infarct-related artery. The mean MI size on LGE CMR was 31.6% of LV mass, with MI size being significantly larger in the IMH+(36.6±13.2% LV) compared to the IMH− group (19.7±10.9% LV). Similar observations were made with respect to MI transmurality with 80.3±7.9% (IMH+) vs. 64.0±12.4% (IMH−), p<0.001 demonstrating LGE reaching the subepicardium. MVO was observed as defects within the area of delayed enhancement in 51 patients, which involved a median volume of 1.58% of the LV mass (IQR 0.64 to 4.10%). Intramyocardial hemorrhage, which evident in 45 patients encompassed a median of 4.6% of LV mass (IQR 2.2 to 6.0%). CMR Findings in STEMI Patients—Myocardial Tissue Characteristics and Cardiac Function—is summarized in Table 4.

TABLE 4
CMR findings at acute phase and follow-up.

| | Total cohort | No hemorrhage | Hemorrhage | |
|---|---|---|---|---|
| | (n = 64) | (n = 19) | (n = 45) | p value |
| Acute phase (n = 64) | | | | |
| LV ejection fraction (%) | 46.4 ± 8.5 | 53.8 ± 4.4 | 43.3 ± 7.8 | <0.001* |
| LV end-diastolic volume | 84.8 ± 13.9 | 80.5 ± 11.1 | 86.6 ± 14.6 | 0.11 |
| index (ml/m²) | | | | |
| LV end-systolic volume | 45.9 ± 12.6 | 37.2 ± 6.8 | 49.5 ± 12.7 | <0.001* |
| index (ml/m²) | | | | |
| Hemorrhagic volume | | 0 | 4.61 (2.18-5.96) | / |
| (% LV) | | | | |
| Hemorrhagic T2* value | 26.5 ± 14.7 | 45.7 ± 11.5 | 18.5 ± 5.7 | <0.001* |
| (ms) | | | | |
| Infarct volume (% LV) | 31.6 ± 14.7 | 19.7 ± 10.9 | 36.6 ± 13.2 | <0.001* |
| Late MVO volume | 2.34 (0.86-4.58) | 0.0 (0.0-0.85) | 3.25 (1.89-5.92) | <0.001* |
| (% LV) | | | | |
| Transmurality (%) | 75.5 ± 12.0 | 64.0 ± 12.4 | 80.3 ± 7.9 | <0.001* |
| Six-month follow up | (n = 41) | (n = 10) | (n = 31) | |
| (n = 41) | | | | |
| LV ejection fraction (%) | 47.5 ± 9.0 | 56.8 ± 2.4 | 44.6 ± 8.3 | <0.001* |
| LV end-diastolic volume | 85.3 ± 16.8 | 76.5 ± 9.0 | 88.1 ± 17.8 | 0.06 |
| index (ml/m²) | | | | |
| LV end-systolic volume | 45.7 ± 15.9 | 33.1 ± 4.7 | 49.8 ± 16.2 | <0.001* |
| index (ml/m²) | | | | |
| Infarct volume (% LV) | 22.5 ± 10.8 | 10.3 ± 6.1 | 26.5 ± 8.8 | <0.001* |
| Transmurality (%) | 69.7 ± 13.1 | 52.8 ± 10.4 | 75.2 ± 8.4 | <0.001* |

LV indicates left ventricle;
MVO, microvascular obstruction.
Data as mean ± SD, median (IQR).

Cardiac Troponin Kinetics is Different Between Hemorrhagic and Non-Hemorrhagic STEMI Patients

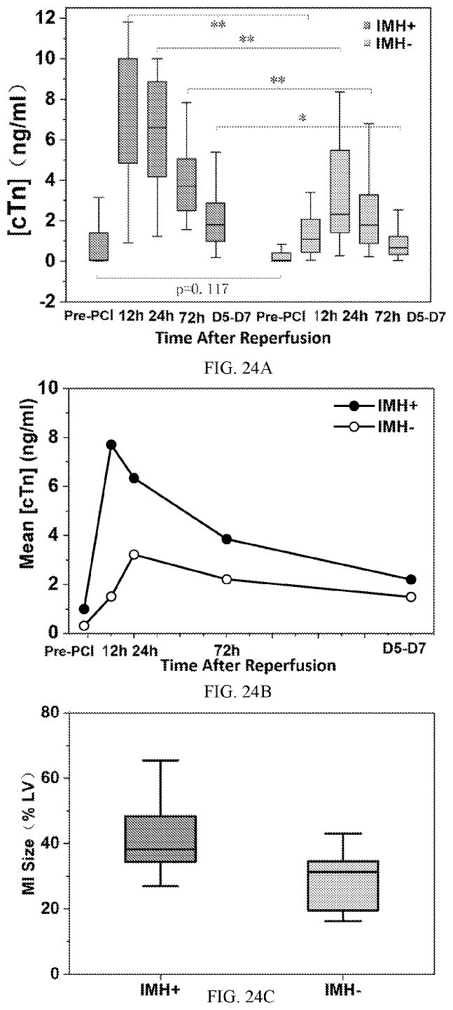

Plasma concentration of cardiac troponin ([cTn]) at initial presentation (before PCI) were not statistically different between IMH+ and IMH− groups (0.6±1.0 vs 0.3±0.5, p=0.22). However, after reperfusion the [cTn] rose significantly more in the IMH+ patients: 12 h (p<0.001), 24 h (p<0.001), 72 h (p=0.001), and 5 to 7 days (p=0.002) compared to IMH− patients (FIGS. 24A-24C), also summarized in Table 5.

TABLE 5
Plasma concentration of cardiac troponin (cTn, ng/ml) in STEMI paitents.

| | Pre-PCI | <12 hr | <24 hr | <72 hr | 5-7 day |
|---|---|---|---|---|---|
| Hemorrhage + | 0.60 | 9.11 | 7.60 | 4.31 | 2.8 |
| Hemorrhage − | 0.30 | 3.26 | 2.75 | 2.31 | 0.98 |

Further, a sharp and significant increase in [cTn] was observed as early as 12 h post PCI in IMH+ patients, and this did not occur in the IMH− patients. The maximal [cTn] was not only significantly lower in IMH− patients at each time point compared to IMH+ patients but also peaked more slowly (peaking ˜24 h post PCI). The differences in the rise in [cTn] were independent of microvascular obstruction (MVO) status, which suggested to us that myocardial necrosis following reperfusion in MIs with IMH evolve differently from those without IMH. To further investigate these observations in a setting free of clinical confounders, we undertook controlled studies in a previously validated large animal model of hemorrhagic MI.

### Cardiac Troponin Kinetics in Canines is Dependent on Hemorrhage Status and Replicates Findings in STEMI Patients

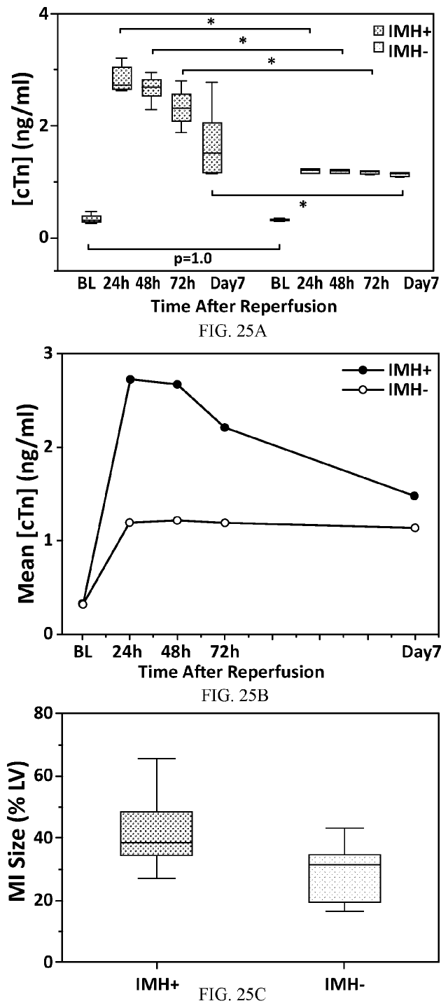

Canines (n=25) underwent controlled ischemia-reperfusion injury in the LAD territory to simulate a mechanically revascularized MI, six died following reperfusion (two <1 hour of reperfusion; and four <3 days of reperfusion) and two animals failed to develop MI. Thus, 17 animals were available for serial studies and were followed for a minimum of eight weeks at regular intervals. Repeat blood sampling was used to evaluate the [cTn] kinetics and repeat CMR was used to evaluate time-dependent changes in myocardial tissue characteristics. Based on post reperfusion CMR, MVO was evident within MI territories in all animals but IMH was observable in only 10 (60%) animals (IMH+ group); the other 7 animals were identified as IMH−. [cTn] levels at baseline, within 24 h, 48 h, 72 h and 7 days post-reperfusion from IMH+ and IMH− animals are shown in FIGS. 25A-25C. Consistent with the [cTn] data observed in STEMI patients, [cTn] at baseline (before reperfusion) were similar between canines in IMH+ and IMH− groups (0.34±0.07 ng/mL vs 0.32±0.02 ng/mL, p=1.00). However, post-reperfusion, [cTn] values showed a robust rise which was significantly higher in the IMH+ group compared to IMH− group: ˜24 h (2.83±0.23 ng/mL vs 1.33±0.38 ng/mL, p<0.001), ˜48 h (2.66±0.21 ng/mL vs 1.21±0.03 ng/mL p<0.001), ˜72 h (2.34±0.32 ng/mL vs 1.19±0.03 ng/mL, p<0.001), and 7 days (1.76±0.52 ng/mL vs 1.14±0.03 ng/mL, p=0.03) (FIGS. 25A and 25B). Furthermore, in the IMH+ group a sharp and significant increase in [cTn] was observed at 24 hrs post-reperfusion but progressively decreased from 24 to 168 hrs post reperfusion. In contrast, in the IMH− group there was no significant difference in [cTn] between 24 hrs and day 7 (1.33±0.50 (˜24 hrs) vs 1.12±0.03 (˜7 days)), p=0.288).

### Time-Lapse Non-Invasive Imaging Shows that the Temporal Evolution of MI Size Post Reperfusion Depends on IMH Status

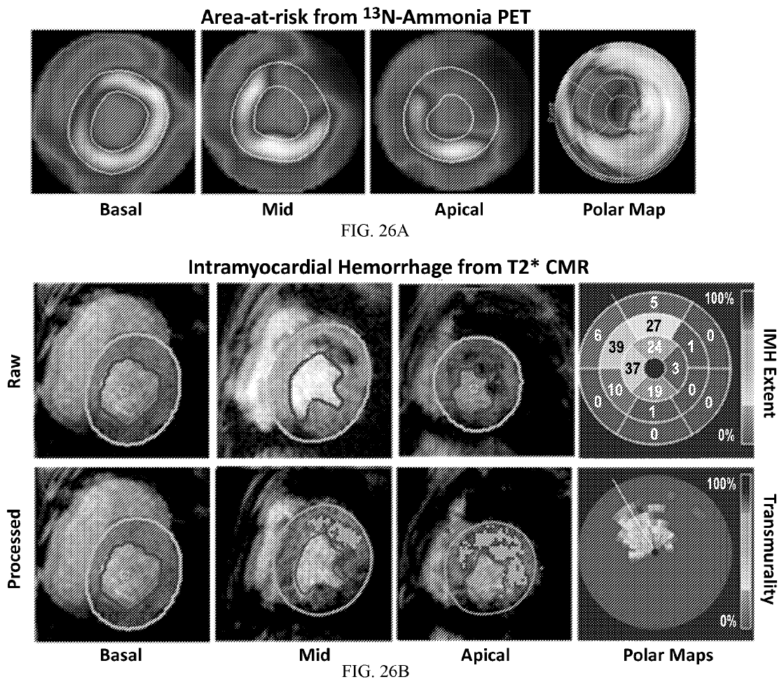

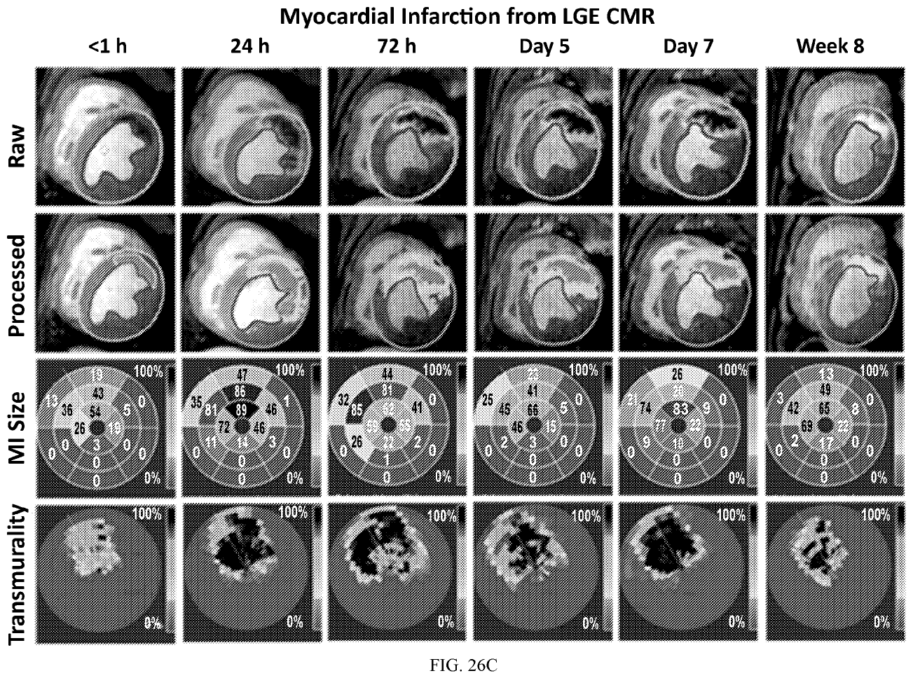

A set of representative images from an animal which developed hemorrhage following ischemia and reperfusion is shown in FIGS. 26A-26C. ¹³N-ammonia PET images were acquired during complete vascular occlusion below the first diagonal of LAD and defined the “area at risk” (AAR). The AAR is the “bloodshed” of a vessel and is representative of the theoretical maximum extent of injury possible for a given occlusion. LGE CMR images acquired within 1 h, as well as at ˜24 h, ˜72 h, ˜5 days, ˜7 days and 8 weeks following reperfusion, along with bull's eye plots depicting MI size and transmurality are shown. T2* CMR images acquired at 72 h post reperfusion, along with bull's eye plot, show the evidence for hemorrhage within the zone of MI identified on LGE. Note the rapid increase in MI size and transmurality between <1 h and ˜24 h of reperfusion.

A similar set of representative images from an animal which did not develop hemorrhage following an equivalent period of ischemia, and then reperfused is shown in FIGS. 27A-27C. Note the modest and slow increase in MI size and transmurality in the cases without hemorrhage compared to the cases with hemorrhage.

The ratio of MI size immediately following reperfusion to the AAR (determined prior to reperfusion) were not different between the hemorrhagic and non-hemorrhagic groups (47.6±6.3% (IMH+) vs. 45.4±9.7% (IMH−), p=0.59).

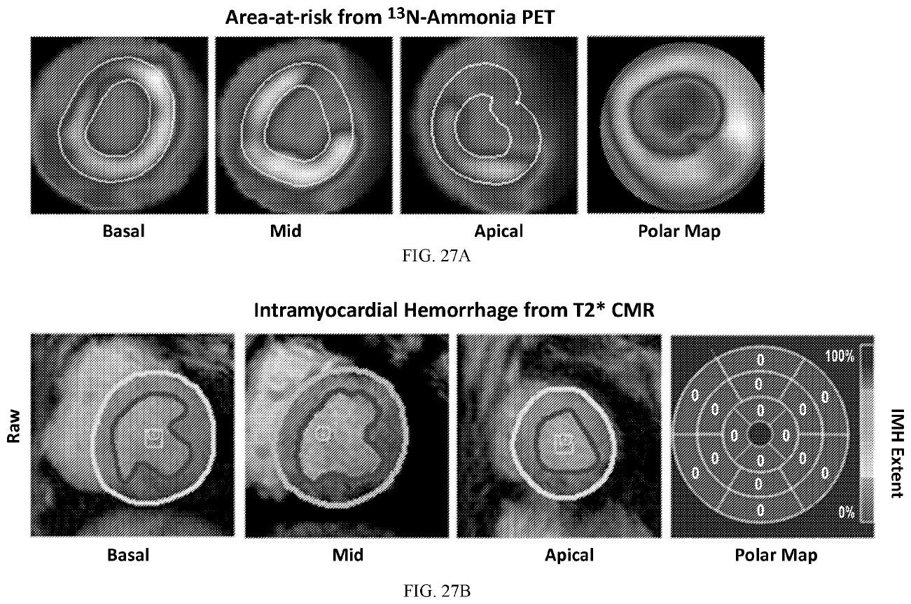

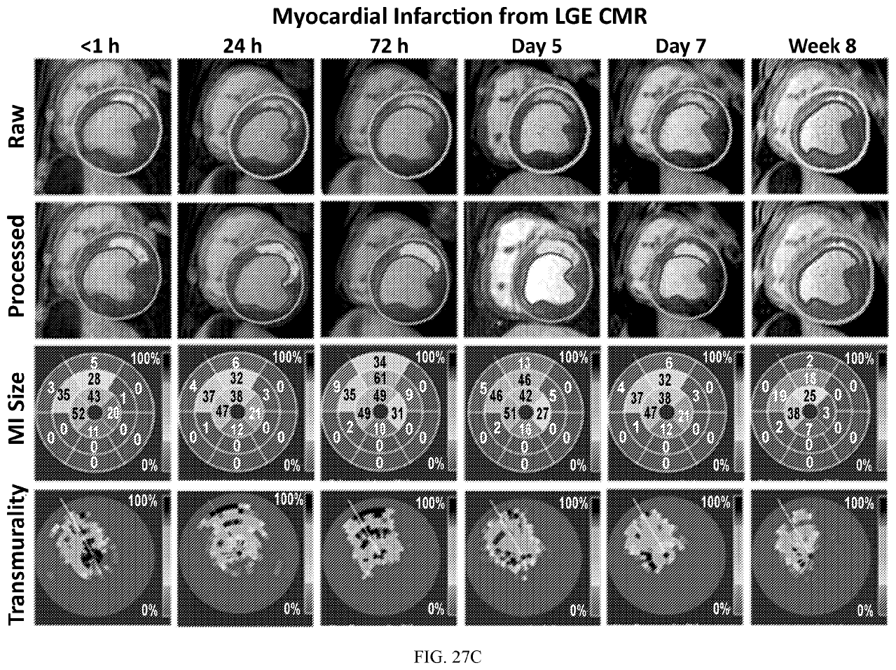

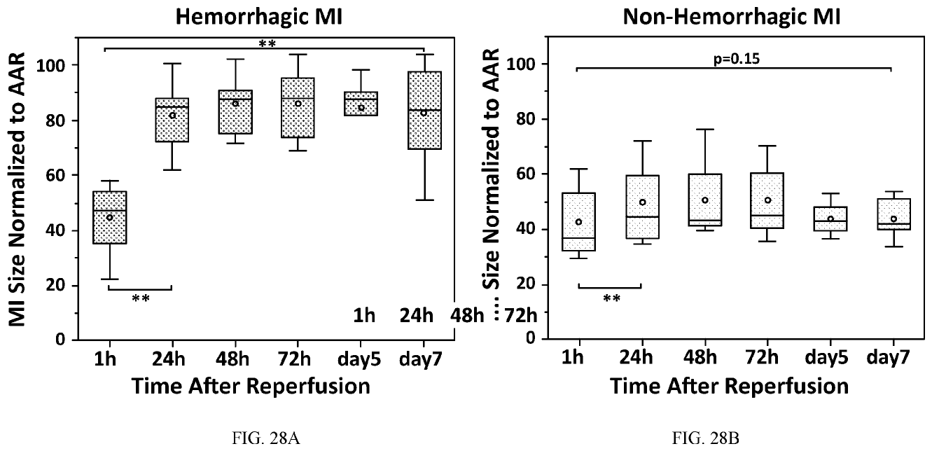

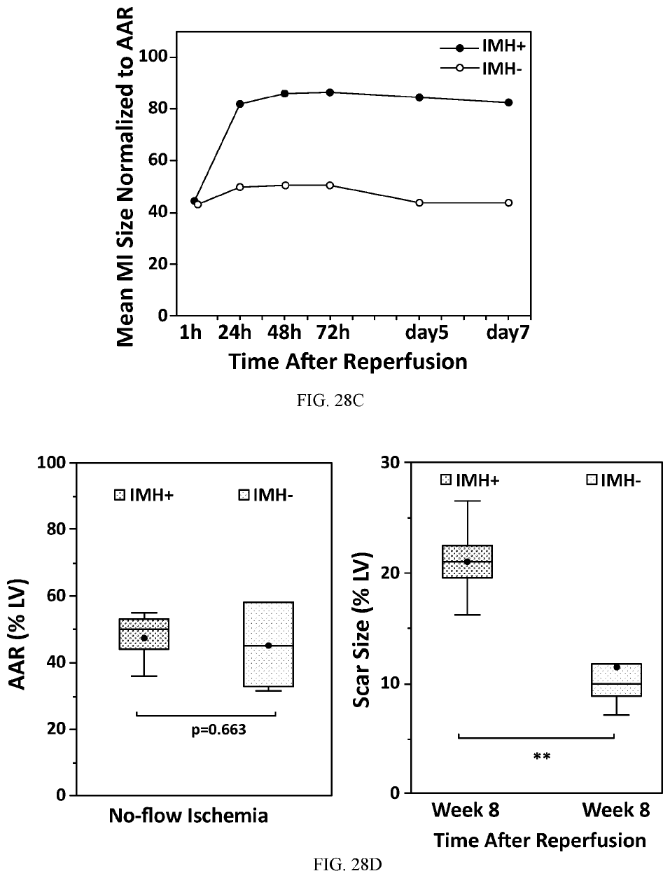

Also within 1 hr of reperfusion, MI size as a % LV mass was not different between the groups (21.0±4.8% (IMH+) vs 18.8±5.70% (IMH−)). However, at all other time points after reperfusion the acute MI size (% LV) of the IMH+ group became significantly higher than that of the IMH− group: at ˜24 hrs: 38.1±7.8% (IMH+) vs 22.0±6.0% (IMH−); at ˜48 hrs: 41.9±5.3% (IMH+) vs 21.4±3.8% (IMH−); at ˜72 h; 43.5±5.8% (IMH+) vs 22.4±6.2% (IMH−) at 5 days: 41.6±6.9% (IMH+) vs. 19.4±2.7% (IMH+); and at 7 days: 37.4±7.8% (IMH+) vs 20.2±6.6% (IMH−). Notably MI size changed significantly in the IMH+ group within the first 72 h of reperfusion but plateaued after 72 h of reperfusion. The mean post reperfusion MI size (% LV) normalized to animal-specific AAR for the IMH+ and IMH− groups as a function of time is shown in FIG. 28A-28C. Further, at week 8, MI size (% LV), as assessed by LGE was 83% greater in the IMH+ group compared to IMH− group (21.0±3.1% (IMH+) vs. 11.5±4.9% (IMH−), p<0.001). Based on serial determination of MI size in every animal from the IMH+ and IMH− groups, ratios of MI size between <1 h of reperfusion and 24 h was computed for each animal and averaged across all animals. This ratio was significantly larger in the IMH+ group compared to the IMH− group (1.80±0.18 (IMH+) vs. 1.18±0.0 (IMH−), p<0.001); and when the same ratio was computed between <1 h and 7 days post reperfusion, similar marked differences were observed 1.79±0.28 (IMH+) vs. 1.04±0.04 (IMH−), p<0.001.

### MIs with Hemorrhage Expand Transmurally in a “Wave Front” Fashion Post Reperfusion and Involve the Epicardium

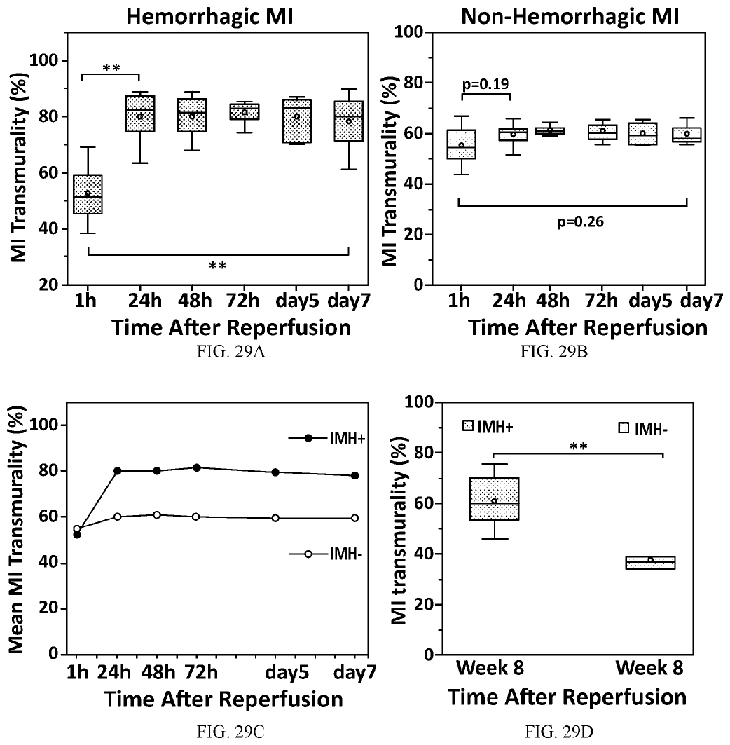

Hemorrhagic infarcts were observed to expand from the endocardium to epicardium as a “wave-front” in the post reperfusion period with infarct transmurality peaking by 24 h and then remaining stable over the first week of reperfusion (see FIG. 26A-26C vs. FIG. 27A-27C). Notably the IMH+ group expanded transmurally from 52.7±9.9% (<1 h) to 77.9±11.1% at (˜24 h), p<0.001. While this was observed only mildly and did not reach statistical significance in the IMH− group: 55.6±3.4% (<1 h) vs. 59.2±1.7% (˜24 h), p=0.284. This pattern of transmural difference, with notable involvement of epicardium in the IMH+ group was evident at week 8 post MI as well (59.8±11.7% vs.37.3±9.0%, p=0.003). See FIG. 29A-29D.

### Rate of MI Expansion Following Reperfusion is Correlated with the Extent of Hemorrhage

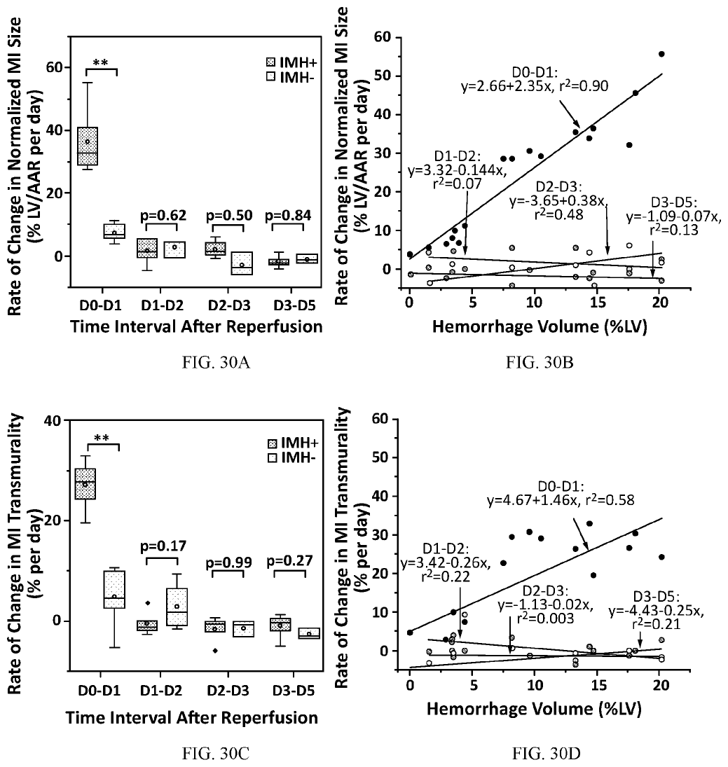

To further characterize the rate of evolution of myocardial injury between the IMH+ and IMH− groups, we investigated the change in MI size and transmurality during the time after the index injury. In contrast to the IMH− group, IMH+ group demonstrated a significantly faster rate of MI expansion between the first 24 hours compared to time windows outside of the first 24 h (24-48 h, 48-72 h, 72 h-5 days, all p<0.001; FIG. 30A). The rate of MI expansion within the first 24 h showed a strong correlation (r2=0.90, P<0.001) with IMH volume, with MI volume increasing at a rate of 2.35% LV per 1% of IMH volume (% LV). There was no significant correlation between MI expansion outside the 24-h period (FIG. 30B). Similar observations were evident with respect to infarct transmurality as well (FIGS. 30C and 30D).

### Hemorrhage Drives the Extent of MVO and Rate of Expansion of MVO Post Reperfusion

All animals, with and without IMH (exposed to indifferent ischemic burden (ischemia time and AAR)), showed evidence for microvascular obstruction throughout the acute phase of MI (1 h-7 days, post reperfusion). However, in the IMH positive group, the extent of MVO (% LV) within the first hour of reperfusion was 4.7-fold greater than in non-hemorrhagic MIs (10.5±4.0% (IMH+) vs 2.3±1.4 (IMH−), p<0.001) but decreased and remained stable thereafter to 7 days. The extent of MVO within the first hour of reperfusion was highly correlated with IMH volume (R2=0.81, p<0.001) but only weakly correlated with MI size within first hour of reperfusion (R2=0.21, p<0.05), indicating that the MVO extent is predominantly modulated by IMH extent than the initial MI size driven by ischemia. Consistent with this, the ratio of the extent of MVO (% LV) to MI size (% LV) was also significantly higher within the first hour of reperfusion in the IMH positive vs. IMH negative group (0.49±0.17 vs 0.12±0.07, p<0.0001) but not at other time points (p>0.01).

### Myocardial Salvage is Time Dependent in MIs with Hemorrhage

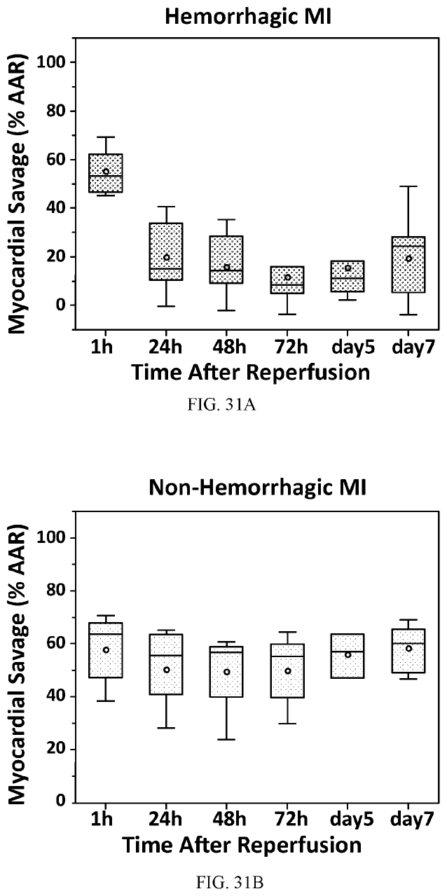

Myocardial salvage in the IMH+ and IMH− groups, calculated as one minus the ratio between MI size as determined by LGE and the associated PET based downstream perfusion bed size (AAR), (or (1−MI size/AAR size)×100%,) immediately post reperfusion (<1 h) were not different (55.2±11.0% vs. 57.5±11.9%, p=0.663). However, in the days following reperfusion the IMH+ group demonstrated a significant loss of salvageable myocardium, with no recovery in net salvageable area over the subsequent week (19.9±18.4% (7 days post reperfusion) vs. 55.2±11.0% (<1 h of reperfusion), p<0.001; FIG. S6). In contrast, the IMH− group showed no significant change in myocardial salvage (56.1±7.3% (<1 h of reperfusion) vs 57.5±11.9% (7 days post reperfusion), p=0.781; FIGS. 31A and 31B). In effect, in spite of having an indifferent salvageable myocardium at <1 h of reperfusion, the salvageable myocardium in the IMH+ group decreased by 2.8-fold in the days following reperfusion. This extensive loss in salvageable myocardium cannot be explained by greater myocardial swelling in the IMH+ group, as wall thickness in MI areas did not differ significantly between the two groups (54.3±7.4 (IMH+) vs. 46.8±13.0 (IMH−), p=0.144). This was also evidenced by the large difference in scar size and infarct transmurality at week 8 between IMH+ and IMH− groups, in spite the fact both groups had indifferent AAR at the terminal point of ischemia. These data collectively demonstrate that while MIs with and without hemorrhage do not differ significantly with respect to apparent size immediately following initial injury (see FIGS. 26A-26C and 27A-27C), the identification of IMH portends a much different evolution of the injury over subsequent days. The presence of IMH predicts both differing kinetics of infarct development, and ultimately worsened myocardial injury.

Our translational study yields new insight into the evolution of tissue injury in the acute phase, especially characterizing the detrimental role of myocardial hemorrhage in ischemia and reperfusion injury. Reperfusion injury was frequent, with MVO occurring in 80% of patients and hemorrhage occurring in 70% of patients—despite of all patients having TIMI 3 flow after reperfusion therapy based on invasive angiographic assessment at the time of PCI—the patients were angiographically not identified as suffering from no-reflow as a reflection of ischemia-reperfusion injury. Possibly, a certain threshold of injury in the microvascular bed may have to occur to lead to a reduction in coronary flow, and that threshold may not have been met immediately at the time of PCI. Refined tissue characterization methods such as cardiac MRI may be more sensitive to detect reperfusion injury as compared to coronary flow measurement at the moment of PCI, and may therefore be necessary if further risk stratification is required.

Reimer and Jennings demonstrated that the two most important determinants of infarct size are the size of vascular bed or amount of myocardium dependent on the culprit lesion, as well as the duration of ischemia to that territory. Cardiomyocyte necrosis evolves in a wavefront-like pattern within this dependent territory, culminating in complete loss of viability if reperfusion therapy is not applied in a timely fashion. Early reperfusion therapy halts the wavefront of necrosis and leads to myocardial salvage, resulting in smaller infarcts, less heart failure and better prognosis. Our investigation adds a reperfusion injury, especially hemorrhage as a third major determinant of infarct size. We observed that hemorrhagic MIs evolved in a wavefront-like fashion resulting in further expansion of infarct size, long after reperfusion therapy had been applied. While microvascular obstruction without hemorrhage was associated with a mild expansion of infarct size around 20%, the wavefront of hemorrhagic MIs rather resembled a tsunami, almost doubling in infarct size (80% increase), thereby nearly completely annihilating the myocardial salvage that was initially achieved with reperfusion therapy. Infarct size, therefore, is not only be determined by ischemic injury alone, but in clinical practice may be the result of ischemic injury plus the iatrogenic reperfusion injury—and the reperfusion hemorrhage may be catastrophic from a perspective of myocardial salvage.

This observation identifies hemorrhage as a new therapeutic target: most earlier research aiming to improve reperfusion focused on platelet aggregation, oxidative stress and endothelial dysfunction. The pathophysiology triggered by hemorrhage may be different and requires further basic science exploration. Importantly, our time lapse approach with serial imaging also identified that infarct expansion driven by hemorrhage evolves mostly within 72 h, thus identifying a critical time window for possible myocardial salvage. If hemorrhage could be avoided or reduced by therapeutic measures in this 72 h time window, infarct expansion could possibly be slowed or halted, resulting in major additional clinical benefit.

We used T2* CMR to identify patients with myocardial hemorrhage 5-7 days of PCI. Our study showed that troponin kinetics are significantly different in hemorrhagic versus non-hemorrhagic MI, and this yields a diagnostic opportunity to differentiate hemorrhagic and non-hemorrhagic MI for daily clinical use. While the troponin kinetics in patients matched that in animals with and without hemorrhage, additional patient studies may further validate our observations in patients. This would require imaging before and at multiple time point after reperfusion which is nearly impossible to perform as such as study in the acute setting would also need to control for multiple parameters (characterization of area-at-risk, true pain-to-balloon time, stability and suitability of patients for repeat imaging in the acute phase of injury, etc.). Given these restrictions, we sought large animal studies to systematically investigate the contribution of IMH toward infarct expansion, all-the-while controlling for known parameters of tissue injury.

### Example 5. Disrupting the Fatty Remodeling of Hearts Following Hemorrhagic Myocardial Infarction with an Intracellular Iron Chelator

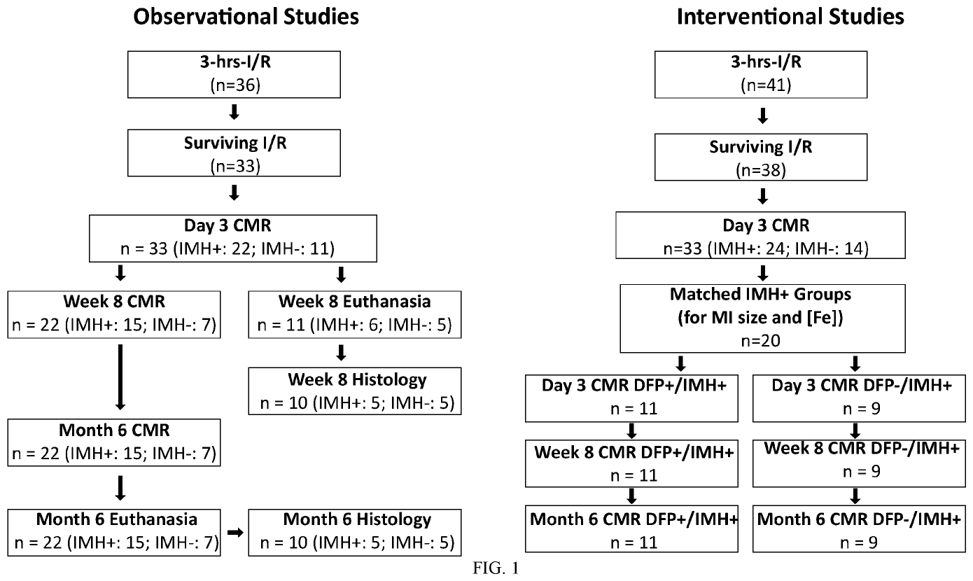

Studies were performed in dogs (n=77; 25-30 kg) subjected to ischemia of 3-hours followed by reperfusion in a series of studies comprising an observational arm and an interventional arm. The observational arm (n=36) was used to serially study the tissue specific changes in iron and fat over a 6-month period with cardiac magnetic resonance imaging (CMR) and histology following reperfused MIs with and without IMH. The interventional arm (n=41) was used to investigate whether an intracellular iron chelator can disrupt the iron deposition, fat infiltration and adverse remodeling in hemorrhagic MIs. The study timeline, animal groups and terminal end points of the various investigation are outlined in FIG. 1.

### Extent of Lipomatous Metaplasia within MI Depends on Acute MI Iron Concentration

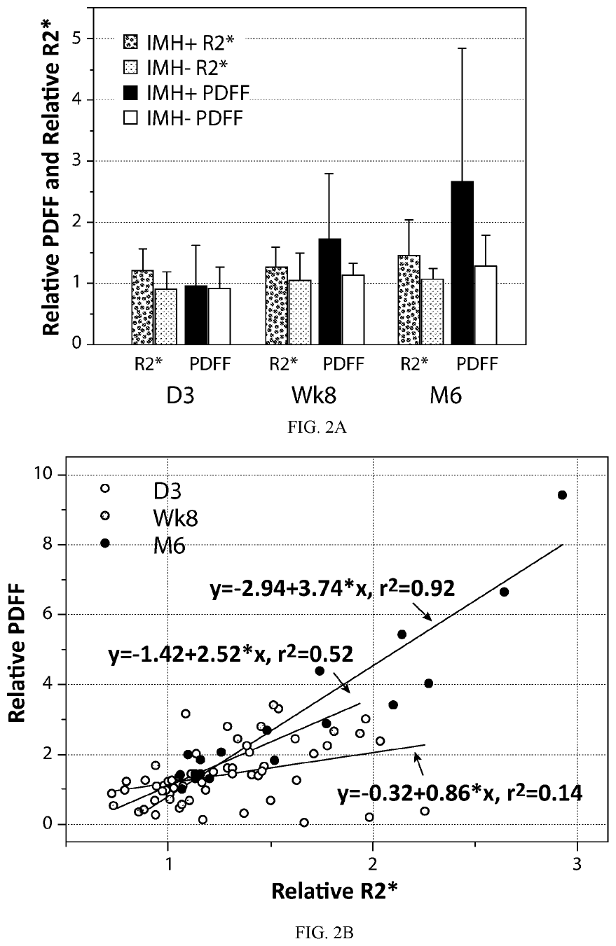

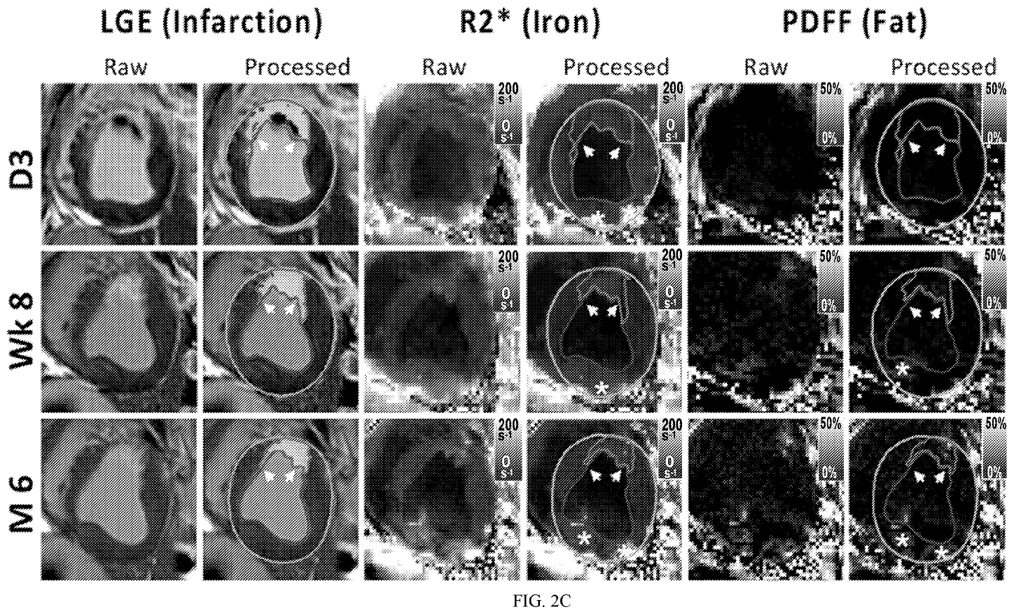

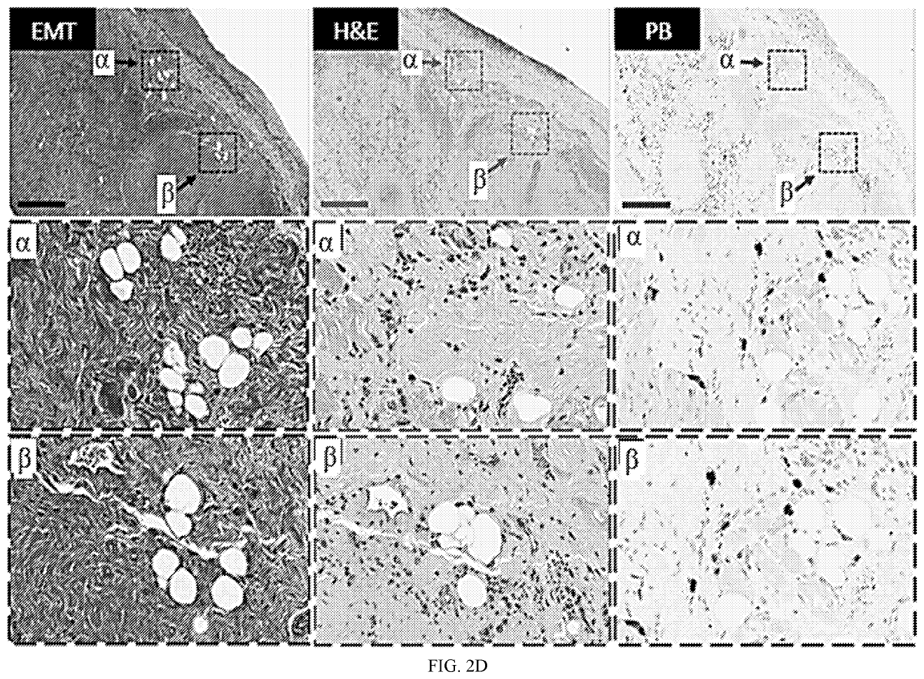

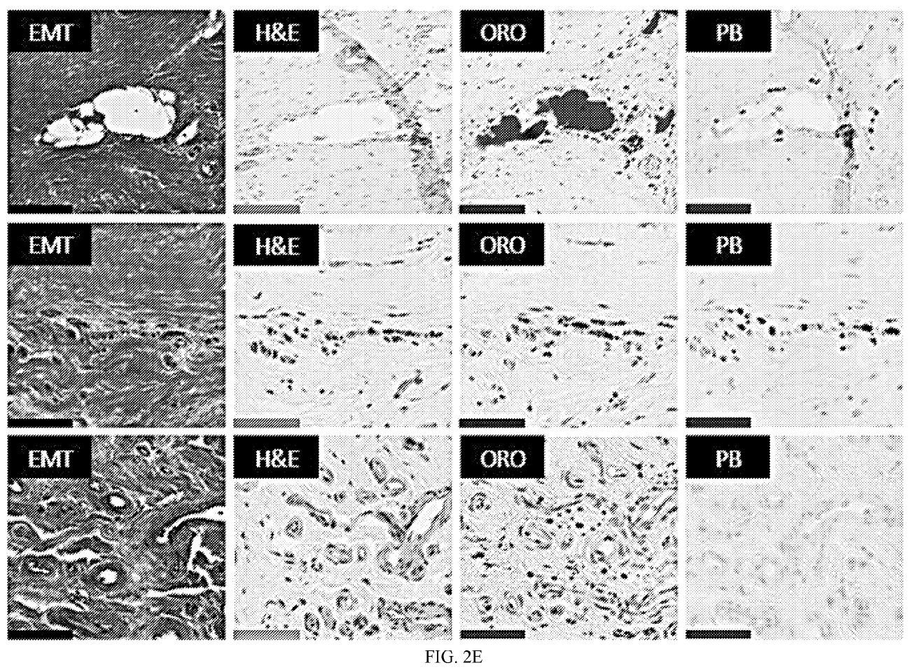

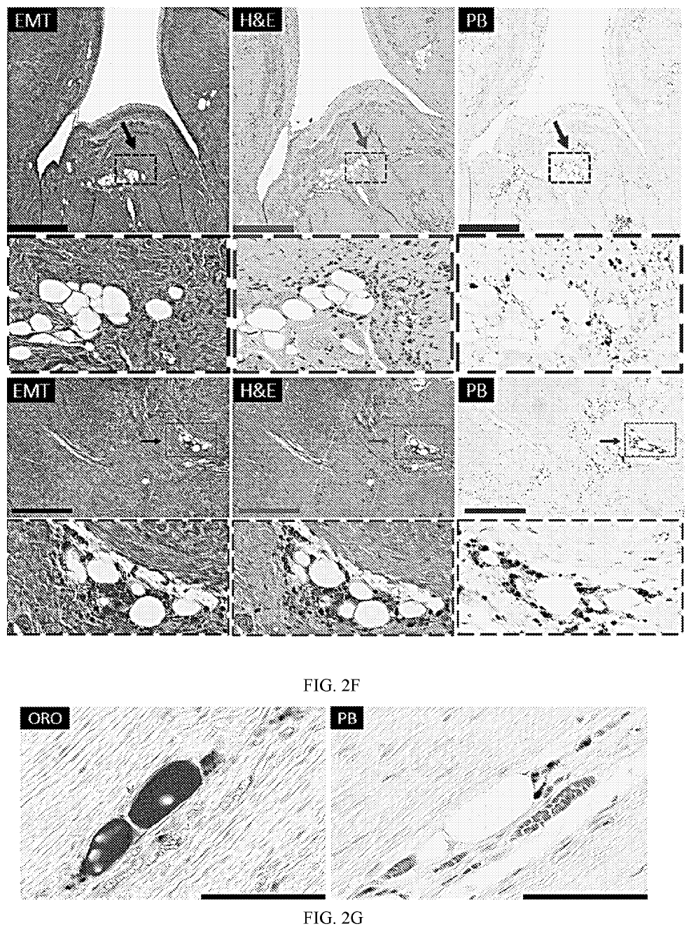

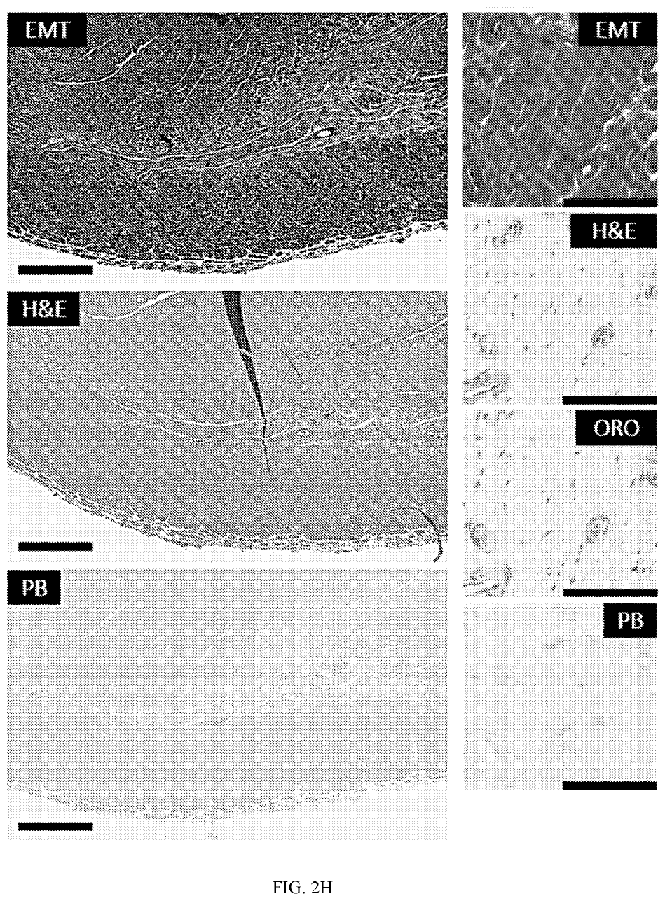

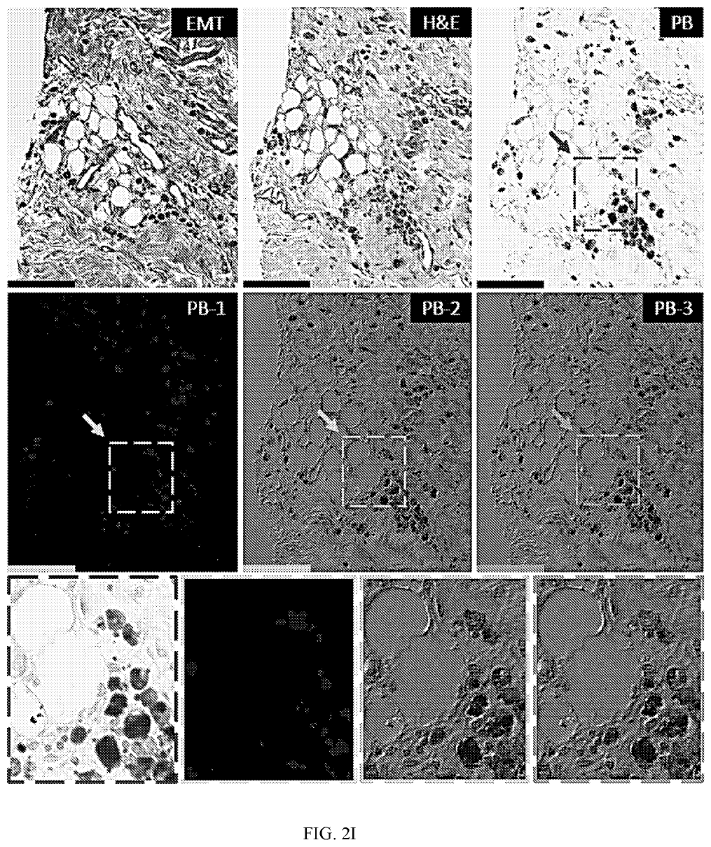

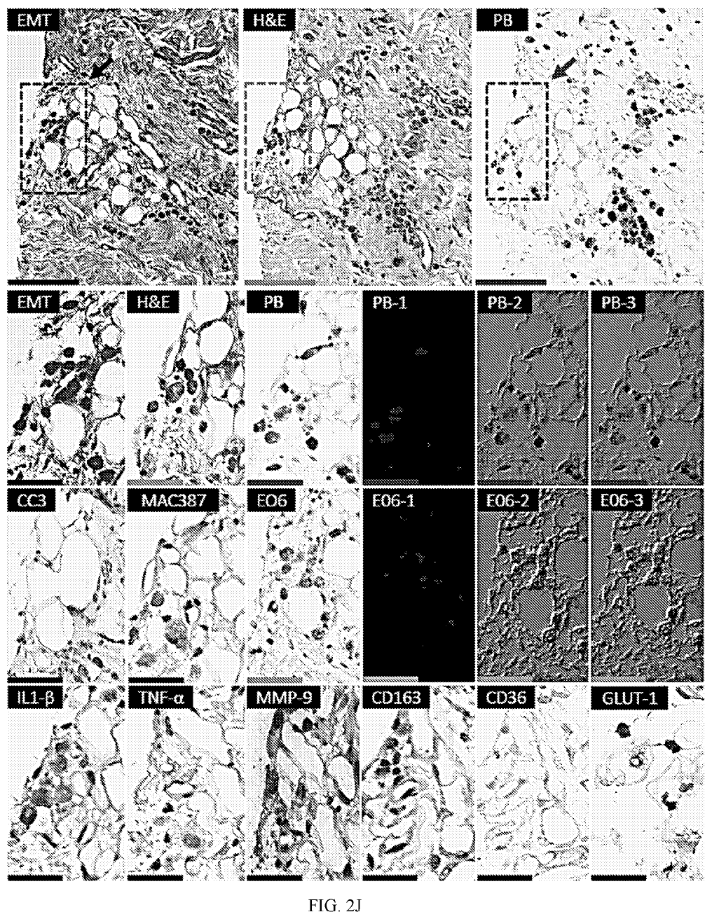

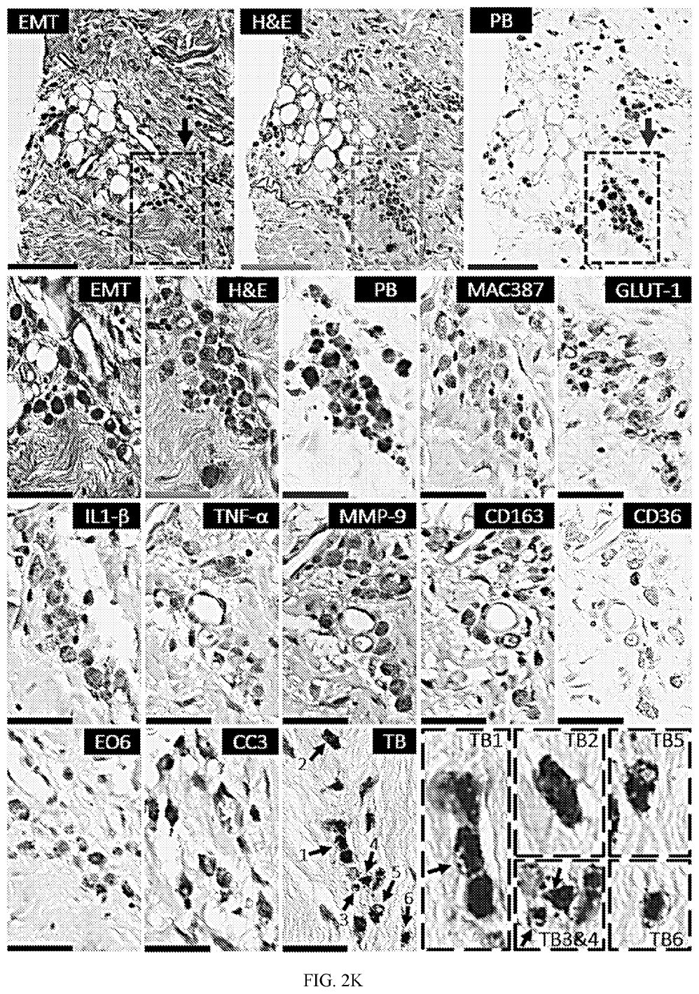

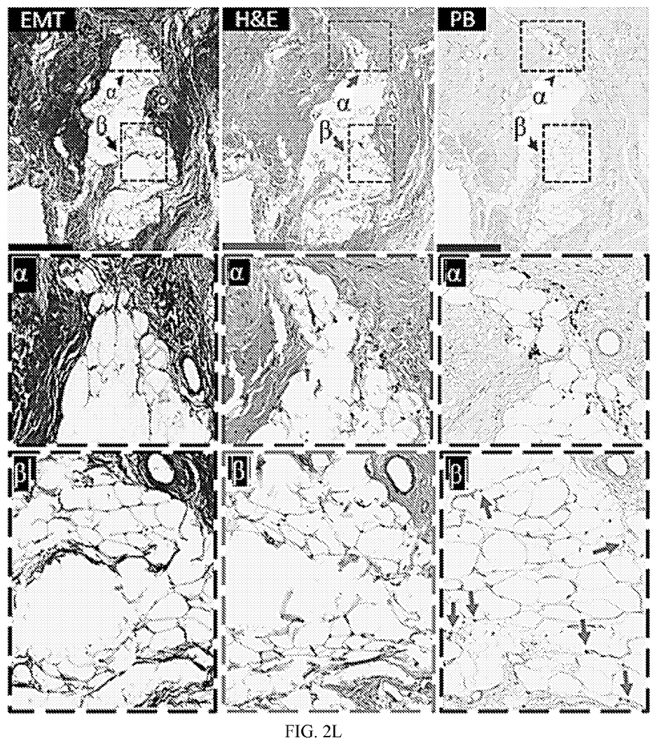

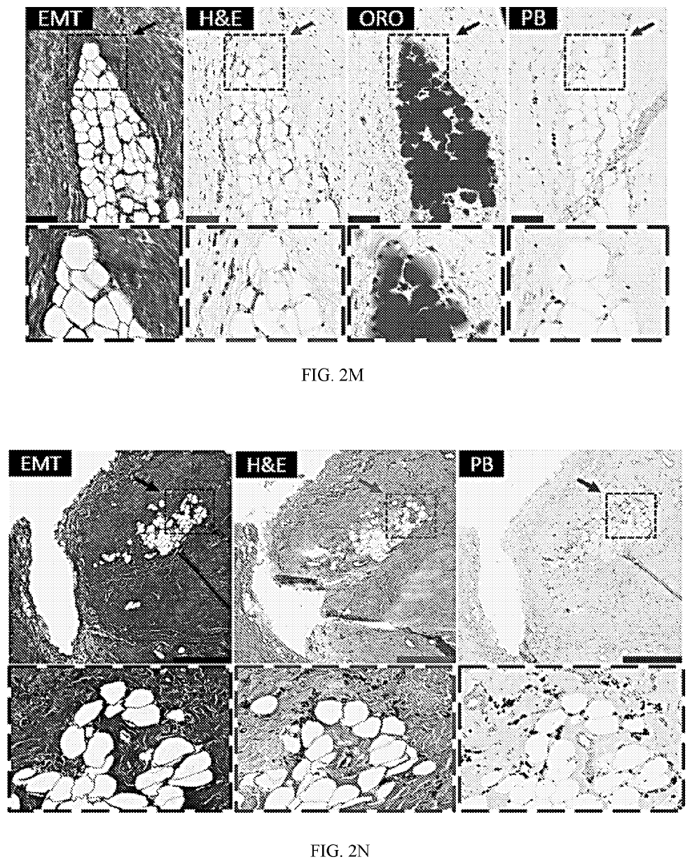

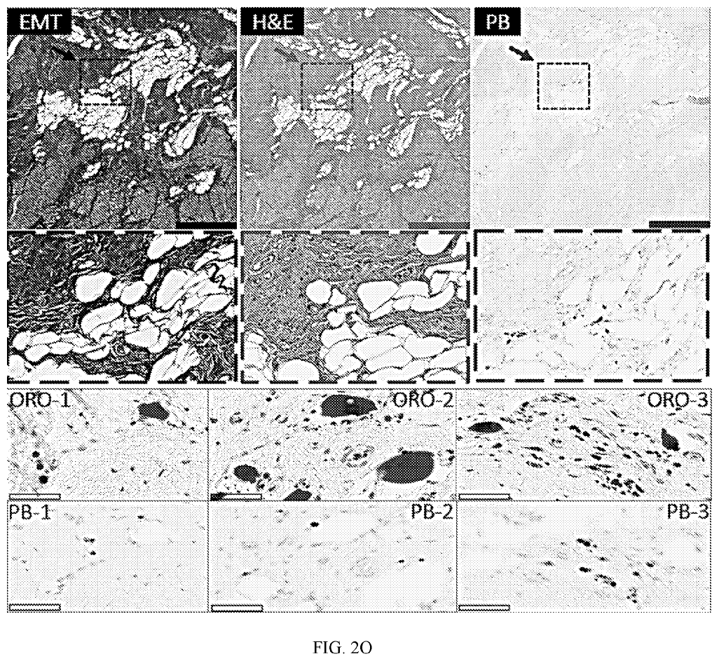

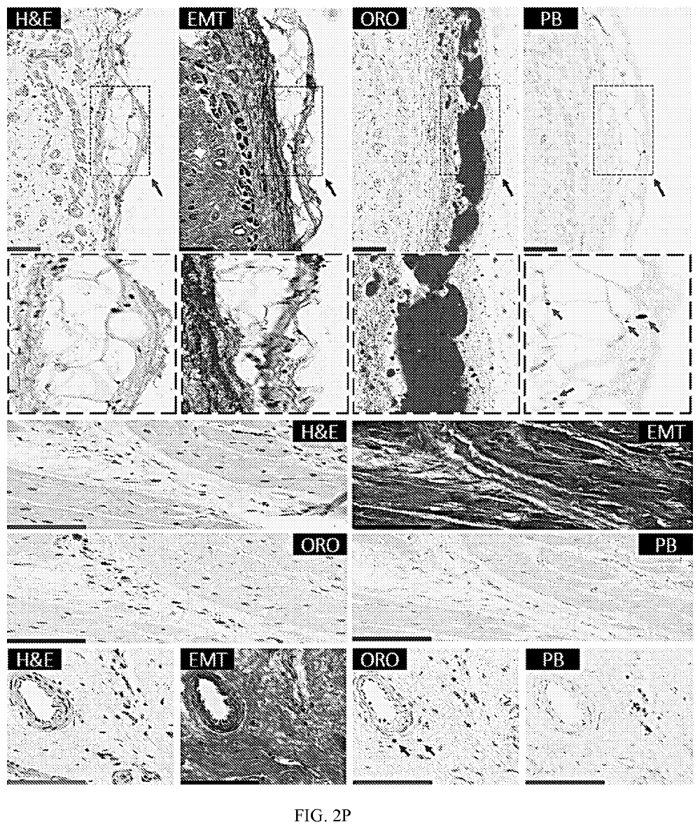

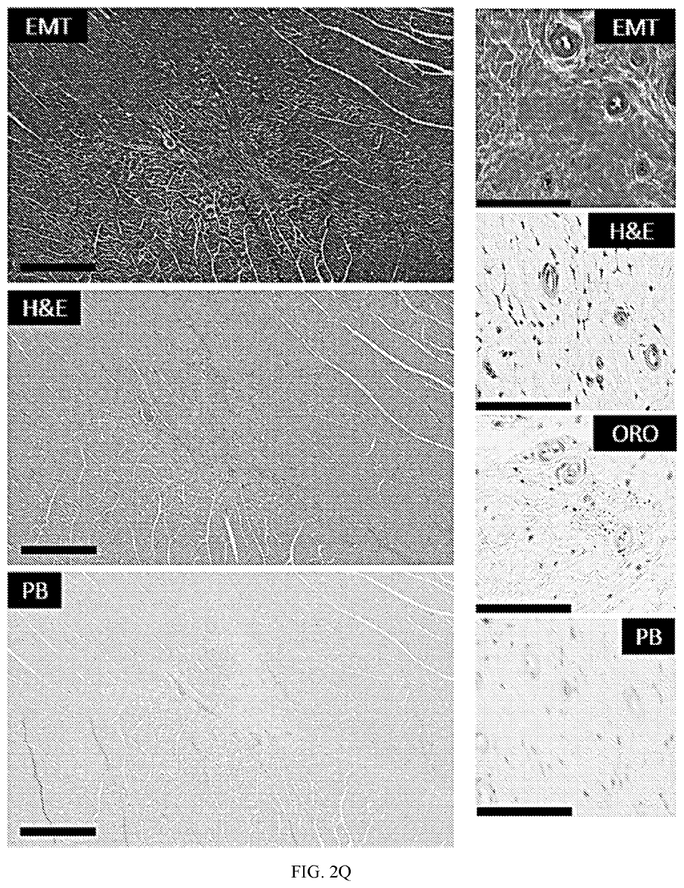

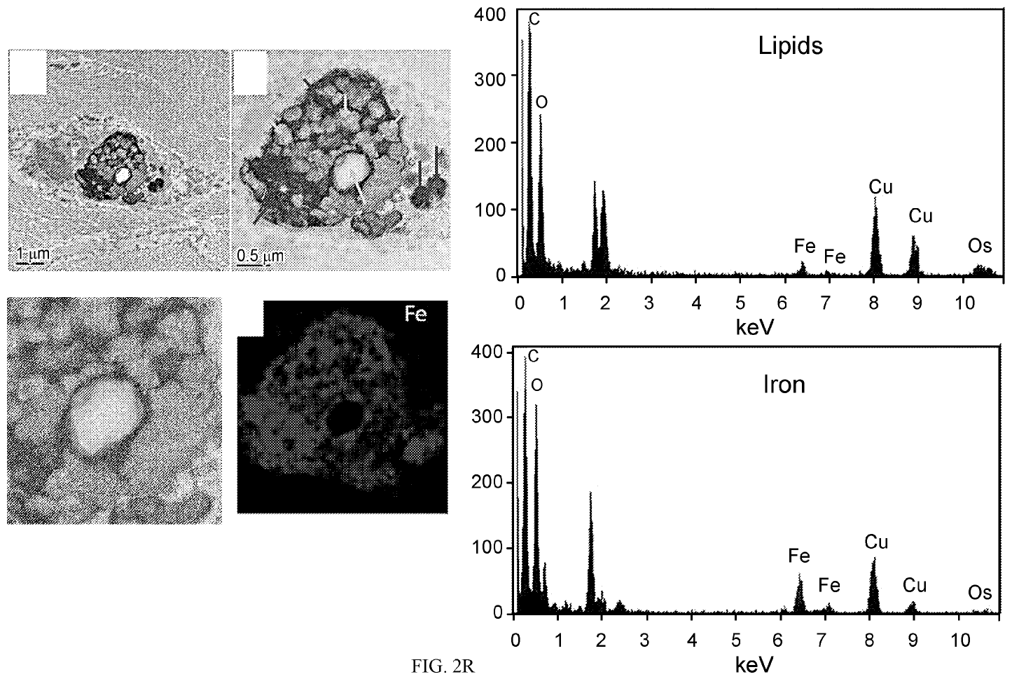

The temporal evolution of fat deposition and its relation to iron within MI were studied using serial CMR in a validated canine model of reperfused MIs with and without hemorrhage. CMR were performed on day 3 (D3), week 8 (Wk8) and month 6 (M6), post MI. Confounder-corrected R2* (or 1/T2*, validated measure of iron concentration ([Fe]) in MI) and proton density fat-fraction (PDFF) maps were constructed and analyzed for relative [Fe] and fat fraction within MI relative to the remote myocardium. Representative findings on R2* and PDFF from an animal with hemorrhagic MI (IMH+) followed over a 6-month period post MI are shown in FIG. 2A. In IMH+ cases, R2* was not different between D3 and M6 (1.20±0.36 at D3, 1.28±0.31 at Wk8, and 1.46±0.57 at M6, p=0.12) suggesting that [Fe] was approximately constant, with only small elevation, between D3 and M6, post MI. However, relative PDFF increased significantly from D3 to M6, from 0.95±0.66 (D3), to 1.72±1.06 (Wk8), to 2.68±2.17 (M6); that is an increase of ˜80% by Wk8 and ˜180% by M6 relative to D3 (p<0.01, for both). In non-hemorrhagic (IMH−) cases, no significant difference was found between the various time points with respect to relative R2* (0.90±0.28 (D3), 1.05±0.20 (Wk8), and 1.07±0.18 (M6), p=0.85) and PDFF (0.92±0.34 (D3), 1.13±0.44 (Wk8), and 1.28±0.50 (M6), p=0.21). Regression analysis showed strong correlations between relative R2* and PDFF in chronic phases of MI (FIG. 2B), with the slope and r² increasing from 0.86±0.33 (r²=0.14; D3) to 2.52±0.55 (r²=0.52; Wk8) to 3.74±0.24 (r²=0.92; M6). This was not the case with IMH− animals: r² were nearly zero (r²=0.04, D3; r²=0.14, Wk8; r²=0.10, M6).

Thus it is concluded that hemorrhagic MIs have elevated and stable level of iron across a 6-month period and that the extent of [Fe] in the acute phase of MI and fat infiltration in the chronic phase are highly correlated, indicating that the iron remnants from hemorrhagic MI may play a role in the extent of fatty infiltrations within the MI zone.

Hemorrhagic MI Mediates a Self-Perpetuating Loop of Iron-Induced Lipid Peroxidation, Foam-Cell Formation, Ceroid Production, Foam-Cell Apoptosis, and Iron Recycling

CMR allowed for serial, gross surveillance of fat deposition and determination of the relation between fat infiltration and iron in the post MI period. To further explore the underpinnings behind the CMR findings and to gather insight into the relation between iron and fat infiltration in MI, studies were performed in the same canine group (from above) by serially sacrificing them at Wk8 and M6. Animals were classified to be IMH+ and IMH− based on R2* CMR and the myocardial tissue was analyzed using histology, immunohistochemistry and transmission electron microscopy.

Early Chronic Phase of MI: IMH+Vs. IMH−

Only IMH+ animals with iron deposits at Wk8 showed evidence of fatty infiltration/LM in the early phase of chronic MI (Wk8). Sparse fat deposits (individual foam cells) were observed in the peripheral and border zones of IMH+ MIs and were exclusively colocalized with Prussian Blue (PB)-stained iron and Oil-Red-O (ORO)-stained extracellular lipids (remnants from necrotic myocardial tissue). See FIGS. 2D-2G. In contrast, animals were negative for iron deposits and also lacked the scarred MI regions undergoing LM (FIG. 2H). Both IMH+ and IMH− animals, however showed the presence of lipids droplets within MI at Wk8 as evidenced by positive ORO staining. These lipid remnants were found in the pen-infarct and border zones of the scarred myocardium. ORO staining confirmed that fat deposits were found exclusively in the scar regions containing both extracellular lipid droplets and persistent iron deposits (Wk8/IMH+/LIPID+/IRON+/LM+).

Thus, neither the lipid-negative/iron-positive (Wk8/IMH+/LIPID−/IRON+/LM−), nor lipid remnant-positive/iron deposit-negative (Wk8/IMH+/LIPID+/IRON−/LM−) regions of hemorrhagic MIs, nor lipid-positive/iron-negative non-hemorrhagic scars (wk8/IMH−/LIPID+/IRON−/LM−) showed evidence of LM. Notably, even small amounts of iron appeared sufficient to result in LM.

Importantly, LM in iron-laden territories was accompanied by ceroid deposition/accumulation as evidenced by a strong/intense autofluorescence (FIGS. 2I, 2J and 2K). Notably, sparse fat cells emerging from the iron-laden regions of hemorrhagic MIs were CD36 positive (foam cell marker) and were strongly colocalized with oxidized phospholipid products (E06-positive lipids), as evidenced by immunohistochemistry. The process of ongoing iron-induced lipid peroxidation was confirmed by confocal microscopy, which demonstrated intense colocalization of autofluorescence signal with PB-stained iron deposits, CD36-stained cells and E06-positive lipids.

Further investigation showed foam cell apoptosis in MI zones of hemorrhagic animals in regions colocalized with iron, ceroid, and newly recruited macrophages. This indicates a self-perpetuating process of iron-induced lipid peroxidation, foam cell formation, ceroid production, foam cell apoptosis, and iron recycling. Iron-laden and E06-rich territories undergoing LM and macrophage/foam cell apoptosis exhibited intense immunostaining for iron scavenger receptor CD163-positive macrophages and proinflammatory macrophage markers (IL-113, TNF-α and MMP-9). Failed switching from proinflammatory glycolytic M1 to anti-inflammatory oxidative M2 phenotype was evidenced by intense GLUT1 immunostaining in siderophages undergoing foam cell transformation. Notably, the iron-laden scar regions undergoing LM were also populated by degranulated mast cells, suggesting the role of mast cells in iron-induced transformation of macrophages to foam cells. Collectively these findings support an important role of hemorrhage iron in LM of MI territories.

Late Chronic Phase of MI: IMH+Vs. IMH−

The process of LM was observed in the late chronic phase of IMH+MI as evidenced in FIGS. 2L-2P. LM was present only in the iron-laden scars at M6 of hemorrhagic MI, while MIs without hemorrhage did not show LM. As at Wk8, individual and mini-clusters of foam cells were observed exclusively at the confluence of post-MI iron and ORO-stained lipid deposits (M6/IMH+/LIPID+/IRON+/LM+), which were typically found in the periphery of hemorrhagic scars. Likewise, ORO-positive/iron-negative (M6/IMH+/LIPID+/IRON−/LM−) and ORO-negative/iron-positive regions of hemorrhagic MIs (M6/IMH+/LIPID−/IRON+/LM−), as well as the ORO-positive/iron-negative non-hemorrhagic MIs (M6/IMH−/LIPID+/IRON−/LM−) (FIG. 2Q) failed to show the presence of foam cells. Histological findings presented in SM were consistent with our observations at Wk8 that even a small amount of iron in the post-MI scar appears to carry a risk of lipid peroxidation, foam cell formation and LM. As at Wk8, individual and mini-clusters of foam cells were colocalized with ceroid. Notably, at M6 we also observed larger fat depots penetrating scar tissue towards its internal core, suggesting LM propagates from the periphery of MI to inside of the MI. Notably, foam cells typically colocalized with iron remnants and lipid droplets along the fat depot periphery (region “a” FIG. 2L), with the core of the growing adipose tissue containing traces of iron deposits (Region “β” in FIG. 2L). However, the presence of ceroid aggregates observed within both the core and the periphery of metaplastic adipose tissue further supports the hypothesis that iron-induced lipid oxidation underlies foam cell formation and progressive LM in the post-MI setting.

To examine the ultrastructural localization of iron and ceroids, the sections of hemorrhagic MIs were studied with transmission electron microscopy (TEM), X-ray spectroscopy. The ongoing process of siderophage-to-foam cell transformation in the chronic MI was also evidenced by TEM and X-ray spectroscopy (FIG. 2R). The intracellular ceroids were observed as clusters of ring structures with electron-dense precipitates within macrophages. To determine the elemental content of the electron-dense precipitates, regions of interest were examined with electron-dense spectroscopy, which showed that the electron-dense precipitates had a strong iron peak. Further, the regions of iron precipitates within the macrophages were highly co-localized with extensive lipid rich regions of the cell. This was not evident in non-hemorrhagic MI zone.

The continued presence of extensive colocalization of ceroid-containing foam cells undergoing apoptosis with iron deposits and newly recruited macrophages at M6 support the notion that iron and lipid recycling drive LM propagation in hemorrhagic MI. There was also evidence of failed switching from proinflammatory to anti-inflammatory macrophage phenotype at M6 as evidenced by intense immunostaining for IL-113, TNF-α, MMP-9 and GLUT-1 in iron-laden regions undergoing macrophage-to-foam cell transformation (FIG. 2S). Further, iron appears to act as a chemoattract of mast cells in the late chronic phase of MI as evidenced by their preferential homing to iron-laden regions undergoing LM. Thus, the observations in the late chronic phase of MI lends further support for the critical role of iron in continually driving the process of LM within hemorrhagic MIs.

### Intracellular Ferric Iron Chelator Deferiprone Reduces Iron within Hemorrhagic MIs and Promotes Beneficial Post MI Left-Ventricular (LV) Remodeling

Further investigated was whether iron with hemorrhagic MIs could be reduced and if so whether it would alter the extent of fat infiltration and alter the course of adverse LV remodeling. Multiple chelation strategies have been investigated in the setting of MI with variable outcomes, but to date no studies have targeted hemorrhagic MIs. The inventor investigated whether an intracellular, trivalent, small molecular weight, iron chelator that has been FDA approved (for other cardiac and non-cardiac indications), deferiprone (DFP) is effective to decrease iron within the MI zone and potentially alter the course of fatty infiltration. This study was carried out in dogs (n=41, FIG. 1) subjected to reperfusion hemorrhage that were randomly assigned to either treatment group (DFP+/IMH+) or control group that did not receive DFP (DFP−/IMH+). CMR was used to determine baseline (pre-MI) cardiac function. CMR was also used to serially assess the changes in [Fe], extent of fat deposition, and its potential impact on structural and functional differences in established markers of LV remodeling using each animal as its own control at D3, Wk8 and M6 for cine (for function), multi-gradient-recalled-echo (for iron) and LGE (for MI size) of full LV. Among the animals that underwent reperfused MI, 20 animals had hemorrhagic MIs with similar MI size and [Fe] at D3 and survived to M6 when randomly assigned to treatment (DFP+/IMH+(n=11)) and untreated control (DFP−/IMH+) groups. The MI size of the groups were: 39.87±5.94% LV (DFP+/IMH+) vs. 38.53±15.15% LV (DFP−/IMH+), p=0.79 at D3.

Effect of Deferiprone on Iron Concentration and Fat Infiltration in Hemorrhagic MIs

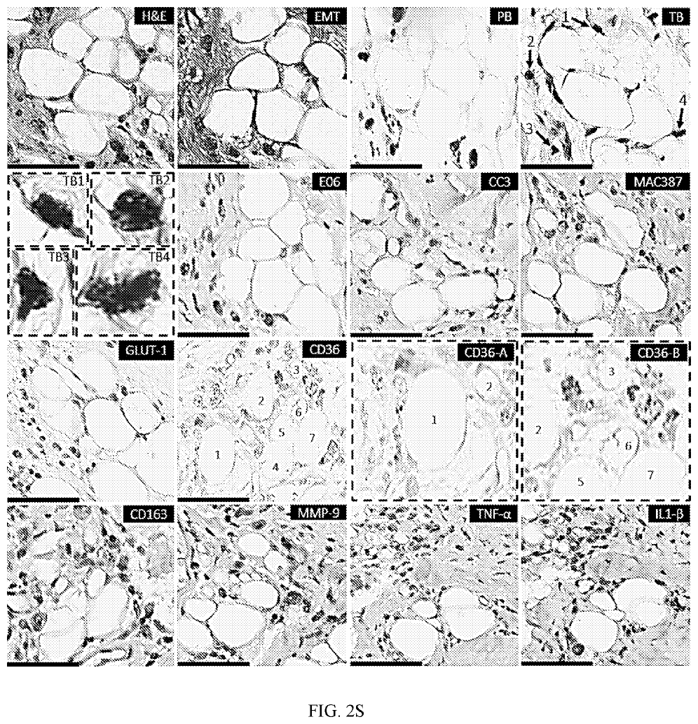

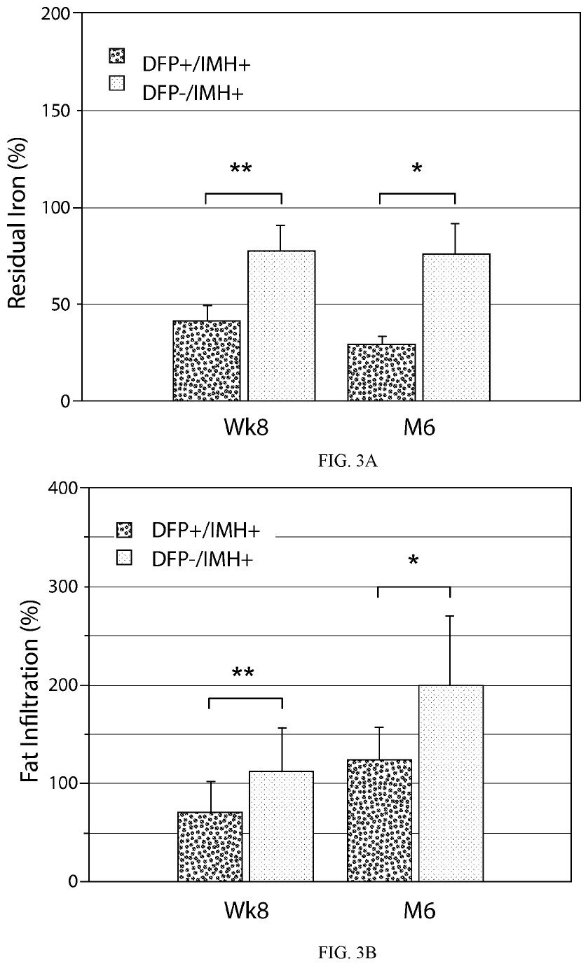

The relative [Fe] between MI and remote myocardium (calculated as R2*MI zone/R2* of remote zone) were: 1.43±0.38 (DFP+/IMH+) vs. 1.44±0.49 (DFP−/IMH+), p=0.96. Animals in the IMH+/DFP+ group received an oral administration of DFP treatment (50 mg/kg, bis in die) to Wk8. Representative confounder-corrected R2* and PDFF maps that were generated using a multi-echo water-fat separation algorithm are shown in FIG. 3C. Residual iron content, computed as relative R2* showed a marked decrease in DFP+/IMH+ group between D3 and Wk8. Relative R2* continued to decrease between Wk8 and M6, albeit at a lower rate (FIG. 3A). Conversely, there was no difference in the relative R2* in the MI zones of DFP−/IMH+ groups between D3 and Wk8 or Wk8 and M6. Importantly, there was marked differences in relative R2* between the treatment and control groups at Wk8 and M6. The effect of DFP on fat infiltration in hemorrhagic MIs was examined using PDFF maps. Relative PDFF in the MI zones did not show an increase in DFP+/IMH+ group between D3 and Wk8 but this in stark contrast to the observation in DFP−/IMIH+ group, which showed an increase in relative PDFF between D3 and Wk8. This discrepancy was more apparent at M6, with significantly lower fat infiltration within the MI zones of DFP+/IMH+ group than DFP−/IMH+ group (FIG. 3B). As such, DFP markedly reduced the iron content within the MI zone and reduced LM with the MI zones, which indicates that iron-rich MI and LM are casually connected.

### Effect of Deferiprone on Structural LV Remodeling Following Hemorrhagic MI

A common structural alteration of hearts that experience adverse LV remodeling in the post MI period is the thickening of remote myocardium and thinning of infarcted myocardium. the time-dependent alterations in diastolic wall thickness of remote and MI segments were investigated, as well as the ratio of infarct to remote wall thickness (i.e. composite remodeling of both the remote and MI segments), and the effect of DFP treatment between D3, Wk8 and M6.

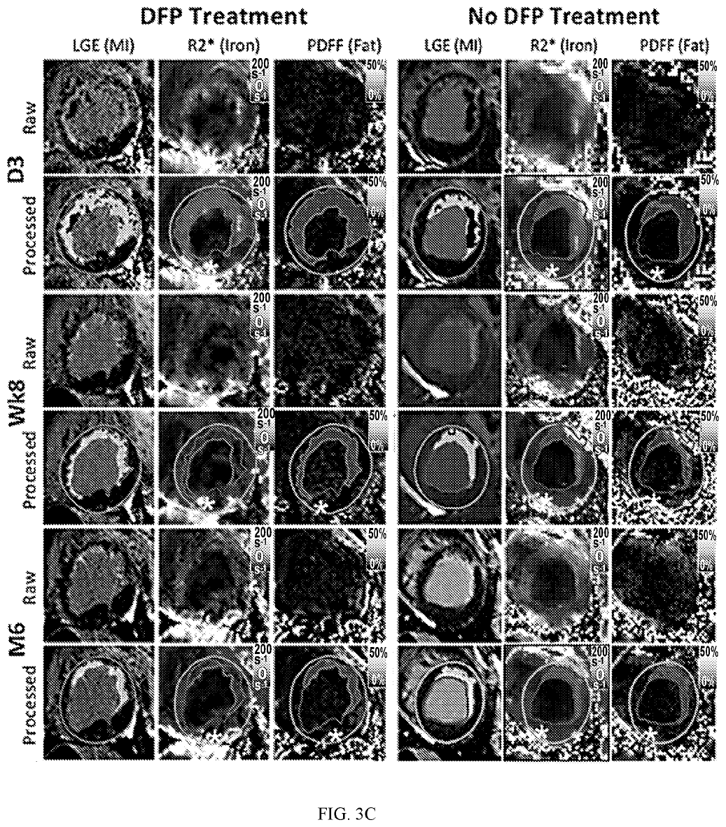

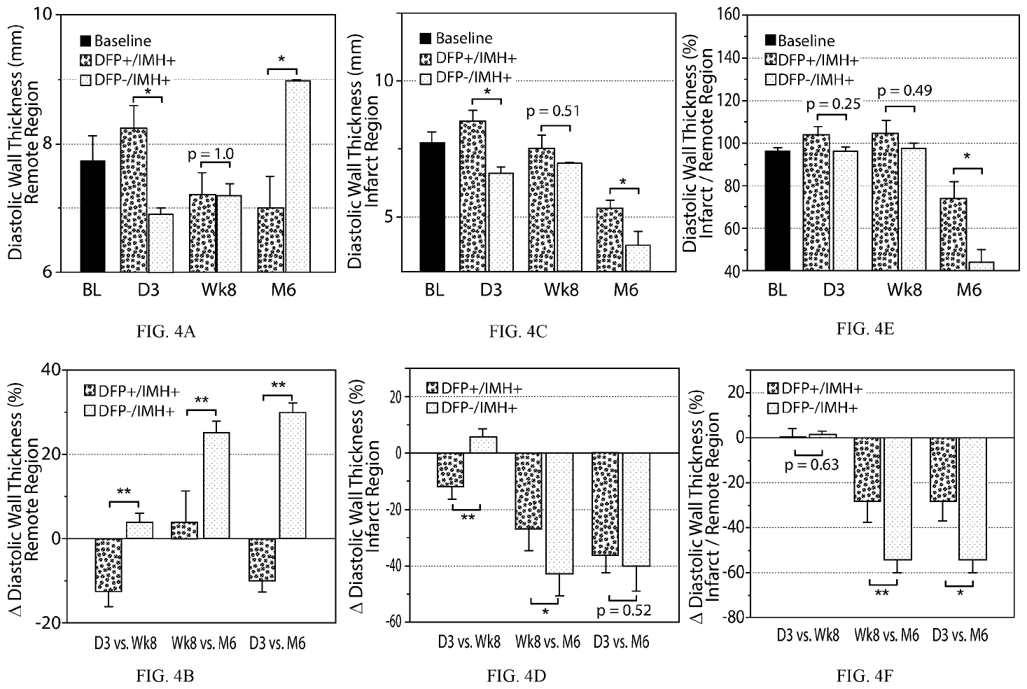

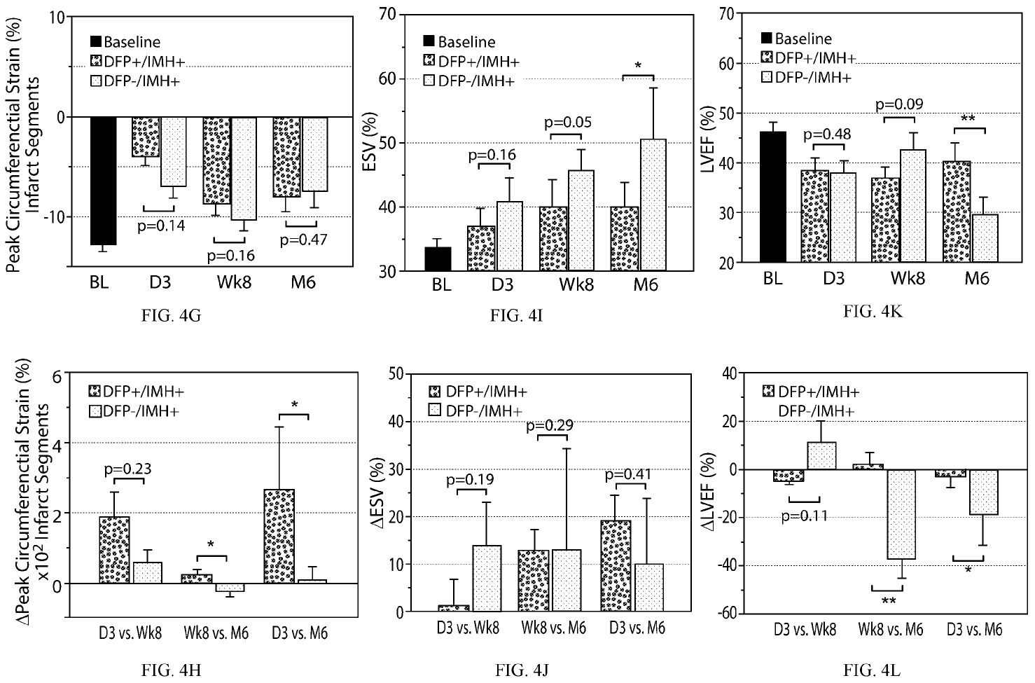

Remote Wall Thickness: The remote segments increased in thickness over the 6-month period in the DFP−/IMH+ group, while it decreased in the DFP+/IMH+ group (FIG. 4A). At M6, the remote wall thickness was significantly larger in DFP−/IMH+ group compared to DFP+/IMH+ group (+28%, p<0.05). Notably, the relative change in wall thickness at Wk8 and M6 (compared to baseline) were substantially greater in the DFP−/IMH+ group compared to DFP+/IMH+ group, with the greatest mean difference in wall thickness was observed between groups between D3 and M6 (p<0.0001). The rate of change of remote wall thickness in the two groups between the two periods D3 to Wk8 versus Wk8 to M6 was different, with remote wall thickness decreasing and then mildly increasing in the DFP+/IMH+ group; whereas the remote wall mildly increased at Wk8 and continued to increase at a faster rate between Wk8 and M6 (FIG. 4B).

Infarct Wall Thickness: MI wall thicknesses steadily decreased over the 6-month period, albeit the decreases were more pronounced between Wk8 and M6 (FIG. 4C). Notably, the infarct wall thickness at M6 was significantly larger in the treated group compared to the untreated group (+34%, p<0.05). Similar to the remote segments, the rate of change of MI wall thickness in the two groups between the two periods D3 to Wk8 versus Wk8 to M6 was different, with MI wall thickness decreasing at a rate smaller than between Wk8 and M6 in the DFP+/IMH+ group; whereas the MI wall mildly increased in thickness at Wk8 and continued to increase at a faster rate between Wk8 to M6 (FIG. 4D).

Infarct-to-Remote Wall Thickness (I:R_{WT}): I:R_{WT} remained constant between D3 and Wk8 but decreased significantly between Wk8 and M6 for both groups (FIG. 4E). However at M6, I:R_{WT} of DFP+/IMH+ group was significantly greater than that of DFP−/IMH+ group (+67%, p<0.05). The rate of change of I:R_{WT} in the two groups between the two periods (D3 to Wk8 and Wk8 to M6) was also different, with I:R_{WT} decreasing at a rate that is significantly faster in DFP−/IMH+ group compared to DFP+/IMH+ group (FIG. 4F).

These studies collectively showed the capacity of DFP to alter anatomical LV remodeling following hemorrhagic MI, which supports the indication that IMH is causally implicated in anatomical remodeling of the heart post MI. Notably, over the period of DFP treatment (i.e., D3 to Wk8), the adverse anatomical remodeling is blunted in the DFP+/IMH+ group compared to the DFP−/IMH+ group. However, once DFP treatment is halted at Wk8, the negative anatomical remodeling resumes, yet with positive anatomical remodeling compared to the untreated group at M6. This indicates that even a modest DFP treatment can reduce the adverse anatomical remodeling that would otherwise ensue in hemorrhagic MIs.

Effect of Deferiprone on Functional Remodeling Following Hemorrhagic MI

Negative structural LV changes in the post MI period leads to adverse functional remodeling of the heart—a defining feature of heart failure. The time-dependent changes in LV functional status between DFP+/IMH+ and DFP−/IMH+ groups were studied. Specifically, changes in peak circumferential strain development in MI segments and global volumetric indices (end-systolic volume and LV ejections fraction), established parameters implicated in adverse functional remodeling of LV, were investigated over a 6-month period in DFP-treated animals in comparison to control animals not receiving DFP treatment.

Peak Circumferential Strain (Peak ε_{c}): Magnitude of Peak ε_{c} increased from D3 to Wk8 in both DFP+/IMH+ and DFP−/IMH+ groups (p<0.001); however, it was not different in DFP+/IMH+ group (p=0.34) but decreased in DFP−/IMH+ group (p<0.01) between Wk8 and M6 (FIG. 5A). The rate of increase in the magnitude of Peak ε_{c} during D3 to Wk8 in DFP+/IMH+ group was higher than in DFP−/IMH+ group, but not significant (p=0.23); however, the magnitude of Peak ε_{c} during the period Wk8 to M6 and D3 to M6 in the DFP+/IMH+ group was significant greater than in the DFP−/IMH+ group (p<0.05, FIG. 5B). These findings indicate that the strain development in fatty tissue is weak and that reduction in LM in the DFP+/IMH+ group permits greater ε_{c} din DFP+/IMH+ group compared to DFP−/IMH+ group.

End-Systolic Volume (ESV): ESV steadily increased from D3 to Wk8 in both DFP+/IMH+ and DFP−/IMH+ groups (p<0.05). However, ESV between Wk8 and M6 was not different in DFP+/IMH+ group (p=0.90), while ESV showed a trend towards increasing between Wk8 and M6 in DFP−/IMH+ group (p=0.19). Notably, ESV of DFP+/IMH+ group was lower than in the DFP−/IMH+ group at both Wk8 and Mo6 (p<0.05, FIG. 5C). The trend in mean rate of increase in ESV during the period D3 to Wk8 in DFP+/IMH+ was markedly lower than in the DFP−/IMH+ group, but this was not statistically significant (FIG. 5D). There was no difference in the rate of change in ESV between DFP+/IMH+ and DFP−/IMH+ groups during Wk8 to M6. These findings show that DFP can positively modulate ESV post hemorrhagic MI.

LV Ejection Fraction (LVEF): In DFP−/IMH+ group, LVEF was lower compared to baseline at D3, increasing by Wk8 and then decreasing well below 40% by M6 (all p<0.05). In comparison, in DFP+/IMH+ group, while LVEF was lower compared to baseline at D3 (p<0.05), it remained unchanged at Wk8 (p=0.12) and increased over 40% by M6 (p<0.03). Notably, at Month 6, the LVEF of DFP+/IMH+ group was markedly higher than in DFP−/IMIH+ group (+36%, p<0.005, FIG. 5E). The rate of change in LVEF between the two period (D3 to W8; and Wk8 to M6) was also very different, with DFP+/IMH+ group showing an increase between D3 to W8 and Wk8 to M6 (p<0.01)), but during the same period DFP−/IMH+ group showed a marked decrease (p<0.0001, FIG. 5F). These findings are consistent with our earlier observations that while fatty infiltration is evident at Wk8, it is between week 8 and month 6 that fat content is substantially increased. This study demonstrated that when LM into MI zone is reduced through DFP treatment, it results in marked positive improvement in LVEF. These findings indicate a causal role of fatty infiltration, driven by iron from IMH, in facilitating adverse functional remodeling of LVs following hemorrhagic MIs.

### Materials and Procedures

Animal Model, Specimen Preparation, and Histology

A total of 77 male mongrel dogs (20-25 kg) were studied according to the protocols approved by the Animal Care and Use Committee. Reperfused myocardial infarction was created as previously described. Briefly, following a left thoracotomy, 2-5 mm segment of the left anterior descending (LAD) artery was dissected from the surrounding tissue just distal to the first diagonal branch, and a suture thread was passed under it as a means of ligature. LAD was temporarily ligated for 3 hrs, followed by reperfusion. After one of reperfusion, the chest wall was closed and the animals were allowed to recover. CMR was performed on day 3 and week 8. In some animals CMR was also performed at 6. Animals were sacrificed either on week 8 or at 6 months post reperfusion and the hearts were explanted. See FIG. 1.

Explanted hearts were sliced into 1 cm thick slices along the short-axis direction from base to apex, and stained with triphenyl tetrazolium chloride (TTC) to histochemically delineate the infarcted territories from viable myocardium. After fixation in 10% glutaraldehyde, the left ventricular (LV) wall samples were cut into two contiguous halves. One half was embedded in paraffin while the other half was immersed in 30% sucrose in 0.1M PBS prior to freezing at −80° C. From paraffin-embedded and frozen blocks, serial 5-μm sections were sliced from representative segments of infarcted and remote areas and were stained with hematoxylin and eosin (H&E) stain for necrosis, elastin-modified Masson's trichrome (EMT) stain for replacement fibrosis (collagen & elastin), Perl's Prussian blue (PB) for iron deposits, and toluidine blue (TB) for mast cell visualization. In addition, Oil-Red-O (ORO) stain was used for determination of lipid/fat content in frozen sections. Representative sections from sections fixed with glutaraldehyde were used electron microscopy.

Cardiac MRI (CMR)—Acquisition, Combined Quantification of Fat and Iron, and Analysis of LV Remodeling

Contiguous, slice-and-resolution matched, short-axis, cine (Repetition time (TR)=3.1 ms; Echo time (TE)=1.6 ms; flip angle=40°; readout bandwidth (BW)=930 Hz/pixel; 25-30 cardiac phases), multiple gradient-recalled echo (mGRE, 6 echoes with TE=3.3-13.3 ms; ATE=2 ms, TR=20 ms; readout bandwidth=1371 H/pixel, flip angle=12°; voxel size=1.5×1.5×8 mm³) and late gadolinium enhancement (LGE, inversion-recovery prepared with balanced steady-state free precession readout, TR=3.42 ms, TE=1.47 ms, readout bandwidth=586 Hx/pixel, flip angle=20°) images were acquired in a whole-body 3T MRI system (Biograph mMR, Siemens Healthineers, Erlangen, Germany) on day 3 (D3), week 8 (Wk8) and month 6 (, post MI. Confounder-corrected R2*(or 1/T2*, an established measure of iron concentration) and proton density fat-fraction (PDFF) maps were reconstructed using a multi-echo water-fat separation algorithm. LGE images were used to identify MI and remote territories. These regions-of-interests were used to determine mean R2* and PDFF, as well relative R2* and relative PDFF estimates (compared to remote areas), of the MI territories. This was performed for all imaging slices at all time points. Structural (end-diastolic sphericity index (EDSI), end-diastolic volume (EDV) and end-systolic volume (ESV)) and functional changes (ejection fraction (EF), wall thickening (WT), wall motion (WM), as well as systolic (ESS), diastolic strain (EDS) and peak (PSS) strain rates) were calculated from cine images. All measurements were normalized to the body surface area. MI size and hemorrhage/iron volume were calculated with respect to the total LV myocardial volume and utilized mean+5SD and mean-2SD threshold cut-offs, respectively.

Immunohistochemistry, Analysis and Confocal Microscopy

For immunostaining, sections were probed with antibodies against markers of canine macrophages as described in Table 6. Slides were digitized on a ScanScope AT (Aperio Technologies, Vista, Calif., USA) instrument and morphometric analysis was performed using Definiens Tissue Studio (Definiens, Parsippany, N.J., USA) software. Predefined stain specific algorithms and classification tools were created utilizing DEFINIENS ECOGNITIONNETWORK LANGUAGE™ to identify positive and negative stained area (area under marker, μm²) within each tissue region in a non-biased method. Areas assessed with Prussian Blue and Oil-Red-O stained regions were regressed. Paraffin sections stained with Perl's Prussian blue as well as the paraffin sections probed with E06 and CD36 antibodies were examined for autofluorescence of ceroid under Leica SP5-X confocal microscope (Leica Microsystems, Wetzlar, Germany).

Transmission Electron Microscopy and X-Ray Spectroscopy

Samples positive for iron from ex vivo sections were further dissected into 1 mm³ cubes and fixed in 2.5% glutaraldehyde (Electron Microscopy Sciences, Hatfield, Pa.) and processed by washing them with dH₂O and a gradual dehydration by using ethanol series (25%, 33, 50, 75, and 3×100% ethanol). The traditional stains for contrast enhancement such as OsO₄ were purposely omitted to preserve the redox state of the biominerals. Samples were then in infiltrated in LR white acrylic resin (Electron Microscopy Sciences), and polymerized at 60° C. for 24 hours. The hardened resin blocks were sectioned on a Leica EM UC6 ultramicrotome using a 45° diamond knife (DiATOME, Hatfield, Pa.). Seventy-nanometer thick sections were collected on copper grids coated with ultrathin carbon on holey carbon support (Pella Inc, Redding, Calif.) and imaged on a Tecnai T-12 TEM (FEI, Hillsboro, Oreg.) with a LaB6 filament, operating at 120 kV. Images were collected digitally with a 2×2K Ultrascan 1000 CCD (Gatan, Pleasanton, Calif.). The elemental mapping was performed on the previously identified areas of interest with Scanning Transmission Electron Microscopy and energy-dispersive X-ray spectroscopy (STEM/EDS) on a JEM-ARM200CF aberration corrected transmission electron microscope operated at 200 kV. The EDS spectra were acquired with beam convergence of 27.5 mrad and beam current of 270 pA using high collection angle Silicon Drift Detector (SDD) (˜0.7 srad, JEOL Centurio). Acquisition and evaluation of the spectra was performed with NSS Thermo Scientific software package.

TABLE 6
Specific targets, associated markers and
antibodies used to probe myocardial tissue.

| Target | Specific Marker | Antibody |
|---|---|---|
| Newly recruited | MAC387 | Abacam ab22506 |
| macrophages | | |
| Macrophage | CD163 | Bioss, bs-2527R |
| scavenger receptor | | |
| Proinflammatory | TNF-α | Abcam, ab6671 |
| cytokine | | |
| Matrix degrading | MMP-9 | Abcam, ab38898 |
| enzyme | | |
| Proinflammatory | IL-1β | Abcam, ab34837 |
| cytokine | | |
| Glucose | GLUT-1 | Biorbyt LLC, |
| transporter | | orb312259 |
| Oxidized | E06 | Avanti Polar Lipids, |
| Phospholipids | | 330001S |
| Foam Cells | CD36 | Sigma-Aldrich, |
| | | AV48129 |
| Apoptosis | Cleaved Caspase-3 | Cell Signaling, |
| | | Asp175 (5A1) |

Iron Chelation Treatment

In a cohort of dogs subjected to reperfused infarction, deferiperone (DFP), a small intracellular (C₇H₉NO₂, molecular weight 139 g/mol) that is known to chelate both intra- and extra-cellular iron (Apopharma Inc., Toronto, ON, Canada), was administered at a dose of 30-40 mg/kg of body weight (BID, PO). The drug treatment commenced immediately following confirmation of reperfusion hemorrhage by T2* CMR on day 3 of MI, and was continued on a daily basis to 8 weeks post MI. Blood samples drawn from the animals were analyzed at 1-month intervals to assess for evidence of agranulosis and anemia.

Statistical Analyses

Statistical analyses were performed using SPSS Statistics (version 21.0, IBM Corporation, Armonk, N.Y.). Shapiro-Wilk test and quantile-quantile plots were used to test the normality of the data. Depending on the normality of the data, analysis of variance or Kruskal-Wallis test along with post-hoc analyses were used to compare measurements among the different groups. Bonferroni correction was used for multiple comparisons. Statistical significance was set at p<0.05.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless otherwise indicated, all numbers expressing quantities should be understood as modified in all instances by the term “about.” The term “about” can refer to ±10% (e.g., ±9%, ±8%, ±7%, ±6%, ±5%, ±4%, ±3%, ±2%, ±1%) of the value being referred to.

Where a range of values is provided, each numerical value between and including the upper and lower limits of the range is contemplated as disclosed herein. It should be understood that any numerical range recited herein is intended to include all sub-ranges subsumed therein. For example, a range of “1 to 10” is intended to include all sub-ranges between and including the recited minimum value of 1 and the recited maximum value of 10; that is, having a minimum value equal to or greater than 1 and a maximum value of equal to or less than 10. Because the disclosed numerical ranges are continuous, they include every value between the minimum and maximum values.

1. A method of treating a subject having been diagnosed with or showing symptoms of myocardial infarction, or minimizing or reducing infarct size in a subject following hemorrhagic myocardial infarction, comprising:
administering to the subject an effective amount of a ferrous iron chelator, an agent that binds heme, or an agent that regulates heme during the acute phase of the myocardial infarction, and
optionally further comprising administering to the subject an effective amount of a ferric iron chelator after the acute phase of the myocardial infarction.

2. (canceled)

3. The method of claim 1, wherein the ferrous iron chelator, the agent that binds heme or the agent that regulates heme is selected from the group consisting of dexrazoxane, 2,2-bipyridl, hinokitiol, hemopexin, a heme oxygenase-1, haptoglobin, albumin, ferritin, α1-microglobulin, al-antitrypsin, glutathione-S-transferase, liver fatty acid binding protein, heme-binding protein 23 (also known as peroxiredoxin), p22 heme binding protein, and glyceraldehyde-3-phosphate dehydrogenase, nuclear factor E2 related factor 2 (Nrf2), feline leukemia virus subgroup C receptor 1a (FLVCR1a), FLVCR2, and ATP-binding cassette subfamily G member 2 (ABCG2) and a combination thereof, and wherein the ferric iron chelator is selected from the group consisting of desferrioxamine, deferiprone, deferasirox, hinokitiol, pyridoxal isonicotinoyl hydrazone, salicylaldehyde isonicotinoyl hydrazone, and a combination thereof.

4. (canceled)

5. The method of claim 1, wherein the administration during the acute phase is within 3 days of onset of the myocardial infarction or characterized by evidence of no ferric iron in or near the myocardium infarct; immediately following onset of myocardial infarction; before, during and/or immediately following reperfusion; or immediately following hemorrhage; and wherein the administration of the ferric iron chelator after the acute phase is performed after 3 days of onset of the myocardial infarction or characterized by evidence of presence of ferric iron in or near the myocardium infarct.

6. (canceled)

7. (canceled)

8. The method of claim 1, wherein the subject has had reperfusion before the administration of the ferrous iron chelator, the agent that binds heme, the agent that regulates heme or the ferric iron chelator.

9. The method of claim 1, wherein the administration of the ferrous iron chelator, the agent that binds heme, or the agent that regulates heme is before reperfusion.

10. The method of claim 1, further comprising selecting a subject having had reperfusion after myocardial infarction, before the administration of the ferrous iron chelator, the agent that binds heme, or the agent that regulates heme in the acute phase.

11. The method of claim 1, further comprising selecting a subject having had intramyocardial hemorrhage after myocardial infarction, before the administration of the ferrous iron chelator, the agent that binds heme, or the agent that regulates heme in the acute phase.

12. (canceled)

13. The method of claim 1, wherein the administration is via oral route, intravenous route, intracoronary route, or the ferrous iron chelator, the agent that binds heme or the agent that regulates heme is incorporated within a stent configured to recanalize occluded coronary artery of the subject.

14. The method of claim 1, wherein the method comprises administering the ferric iron chelator after the acute phase of the myocardial infarction, and the ferric iron chelator is administered over a time period of 1 week, 2 weeks, 3 weeks, 4 weeks, 2 months, 3 months, 6 months or longer.

15. The method of claim 1, further comprising administering an anti-inflammatory agent to the subject in the acute phase of the MI, or after the acute phase of the MI.

16. The method of claim 15, further comprising administering colchicine to the subject in the acute phase of the MI, or after the acute phase of the MI.

17-20. (canceled)

21. A method for reducing myocardial infarct size, and/or inhibiting expansion of the myocardial infarct size, in a subject in need thereof, comprises:
administering a composition comprising an effective amount of a ferrous iron chelator, an agent that binds heme, an agent that regulates heme, or a combination thereof, during the acute phase or within 3 days of the onset of myocardial infarction;
measuring a blood level of troponin or cardiac troponin of the subject before and after coronary re-vascularization or reperfusion therapy, or at two or more time points after the coronary re-vasucularization or the reperfusion therapy; and
administering a treatment to the subject to control hemorrhage from the cardiac chamber of the subject, when the blood level of troponin or cardiac troponin rises within 30 minutes to 12 hours following the coronary re-vascularization or the reperfusion therapy, which is at least 3 times higher compared to the level in the subject before the coronary re-vascularization or the reperfusion therapy; or no treatment to control hemorrhage is administered to the subject, when the blood level of troponin or cardiac troponin within 30 minutes to 12 hours following the coronary re-vascularization or the reperfusion therapy is not higher, or less than 3 times higher, compared to the level in the subject before the coronary re-vascularization or the reperfusion therapy.

22. The method of claim 21, wherein the subject is a subject with ST-elevation myocardial infarction.

23. The method of claim 21, wherein the subject is a subject in need of or having had a reperfusion therapy.

24. The method of claim 21, wherein the subject is a subject at risk of developing intramyocardial hemorrhage.

25. The method of claim 21, further comprising determining the subject has intramyocardial hemorrhage whose blood level of troponin or cardiac troponin within 30 minutes to 12 hours following the coronary re-vascularization or the reperfusion therapy is at least 7 times, 6 times, 5 times, 4 times or 3 times higher than that before the coronary re-vascularization or the reperfusion therapy.

26. The method of claim 21, further comprising administering an effective amount of a ferric iron chelator to the subject in the chronic phase of the myocardial infarction or after 3 days from the onset of symptoms of the myocardial infarction.

27. The method of claim 21, wherein the subject is a human subject.

28. A method for determining the presence of hemorrhagic myocardial infarction in a subject undergoing or having undergone a reperfusion therapy, comprising measuring a level of troponin from a biological sample of the human subject over time following the reperfusion therapy, wherein the level of troponin peaks within 18 hours following the reperfusion therapy, the level of troponin increases by at least 1.5 ng/mL within 18 hours following the reperfusion therapy compared to a level before the reperfusion therapy, or the level of troponin increases by a rate of at least 0.4 ng/mL/hr within 12 hours following the reperfusion therapy.

29. A method for treating hemorrhagic myocardial infarction in a subject, and/or mitigating infarct expansion in a subject with hemorrhagic myocardial infarction comprises: administering an effective amount of a ferrous iron chelator, an agent that binds heme, or an agent that regulates heme during the acute phase of the myocardial infarction to the subject, optionally further administering an effective amount of a ferric iron chelator after the acute phase to the subject, wherein the subject has been determined with presence of hemorrhagic myocardial infarction according to the method of claim 28.
